SEQUENCE LISTING (26 sequences)

SEQ ID NO: 1 - protein, 919 aa, Homo sapiens
MKVARFQKIPNGENETMIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVSSTSSSFLEV

SEQ ID NO: 2 - protein, 1042 aa, Homo sapiens
MENAHTKTVEEVLGHFGVNESTGLSLEQVKKLKERWGSNELPAEEGKTLLELVIEQFEDLLVRILLLAACISFVLAWFEEGEETITAFVEPFVILLILVANAIVGVWQERNAENAIEALKEYEPEMGKVYRQDRKSVQRIKAKDIVPGDIVEIAVGDKVPADIRLTSIKSTTLRVDQSILTGESVSVIKHTDPVPDPRAVNQDKKNMLFSGTNIAAGKAMGVVVATGVNTEIGKIRDEMVATEQERTPLQQKLDEFGEQLSKVISLICIAVWIINIGHFNDPVHGGSWIRGAIYYFKIAVALAVAAIPEGLPAVITTCLALGTRRMAKKNAIVRSLPSVETLGCTSVICSDKTGTLTTNQMSVCRMFILDRVEGDTCSLNEFTITGSTYAPIGEVHKDDKPVNCHQYDGLVELATICALCNDSALDYNEAKGVYEKVGEATETALTCLVEKMNVFDTELKGLSKIERANACNSVIKQLMKKEFTLEFSRDRKSMSVYCTPNKPSRTSMSKMFVKGAPEGVIDRCTHIRVGSTKVPMTSGVKQKIMSVIREWGSGSDTLRCLALATHDNPLRREEMHLEDSANFIKYETNLTFVGCVGMLDPPRIEVASSVKLCRQAGIRVIMITGDNKGTAVAICRRIGIFGQDEDVTSKAFTGREFDELNPSAQRDACLNARCFARVEPSHKSKIVEFLQSFDEITAMTGDGVNDAPALKKAEIGIAMGSGTAVAKTASEMVLADDNFSTIVAAVEEGRAIYNNMKQFIRYLISSNVGEVVCIFLTAALGFPEALIPVQLLWVNLVTDGLPATALGFNPPDLDIMNKPPRNPKEPLISGWLFFRYLAIGCYVGAATVGAAAWWFIAADGGPRVSFYQLSHFLQCKEDNPDFEGVDCAIFESPYPMTMALSVLVTIEMCNALNSLSENQSLLRMPPWENIWLVGSICLSMSLHFLILYVEPLPLIFQITPLNVTQWLMVLKISLPVILMDETLKFVARNYLEPGKECVQPATKSCSFSACTDGISWPFVLLIMPLVIWVYSTDTNFSDMFWS

SEQ ID NO: 3 - DNA, 2760 bp, Homo sapiens
  source 1..2760: mol_type = genomic DNA
atgaaggttgcacgttttcaaaaaatacctaatggtgaaaatgagacaatgattcctgtattgacatcaaaaaaagcaagtgaattaccagtcagtgaagttgcaagcattctccaagctgatcttcagaatggtctaaacaaatgtgaagttagtcataggcgagcctttcatggctggaatgagtttgatattagtgaagatgagccactgtggaagaagtatatttctcagtttaaaaatccccttattatgctgcttctggcttctgcagtcatcagtgttttaatgcatcagtttgatgatgccgtcagtatcactgtggcaatacttatcgttgttacagttgcctttgttcaggaatatcgttcagaaaaatctcttgaagaattgagtaaacttgtgccaccagaatgccattgtgtgcgtgaaggaaaattggagcatacacttgcccgagacttggttccaggtgatacagtttgcctttctgttggggatagagttcctgctgacttacgcttgtttgaggctgtggatctttccattgatgagtccagcttgacaggtgagacaacgccttgttctaaggtgacagctcctcagccagctgcaactaatggagatcttgcatcgagaagtaacattgcctttatgggaacactggtcagatgtggcaaagcaaagggtgttgtcattggaacaggagaaaattctgaatttggggaggtttttaaaatgatgcaagcagaagaggcaccaaaaacccctctgcagaagagcatggacctcttaggaaaacaactttccttttactcctttggtataataggaatcatcatgttggttggctggttactgggaaaagatatcctggaaatgtttactattagtgtaagtttggctgtagcagcaattcctgaaggtctccccattgtggtcacagtgacgctagctcttggtgttatgagaatggtgaagaaaagggccattgtgaaaaagctgcctattgttgaaactctgggctgctgtaatgtgatttgttcagataaaactggaacactgacgaagaatgaaatgactgttactcacatatttacttcagatggtctgcatgctgaggttactggagttggctataatcaatttggggaagtgattgttgatggtgatgttgttcatggattctataacccagctgttagcagaattgttgaggcgggctgtgtgtgcaatgatgctgtaattagaaacaatactctaatggggaagccaacagaaggggccttaattgctcttgcaatgaagatgggtcttgatggacttcaacaagactacatcagaaaagctgaatacccttttagctctgagcaaaagtggatggctgttaagtgtgtacaccgaacacagcaggacagaccagagatttgttttatgaaaggtgcttacgaacaagtaattaagtactgtactacataccagagcaaagggcagaccttgacacttactcagcagcagagagatgtgtaccaacaagagaaggcacgcatgggctcagcgggactcagagttcttgctttggcttctggtcctgaactgggacagctgacatttcttggcttggtgggaatcattgatccacctagaactggtgtgaaagaagctgttacaacactcattgcctcaggagtatcaataaaaatgattactggagattcacaggagactgcagttgcaatcgccagtcgtctgggattgtattccaaaacttcccagtcagtctcaggagaagaaatagatgcaatggatgttcagcagctttcacaaatagtaccaaaggttgcagtattttacagagctagcccaaggcacaagatgaaaattattaagtcgctacagaagaacggttcagttgtagccatgacaggagatggagtaaatgatgcagttgctctgaaggctgcagacattggagttgcgatgggccagactggtacagatgtttgcaaagaggcagcagacatgatcctagtggatgatgattttcaaaccataatgtctgcaatcgaagagggtaaagggatttataataacattaaaaatttcgttagattccagctgagcacgagtatagcagcattaactttaatctcattggctacattaatgaactttcctaatcctctcaatgccatgcagattttgtggatcaatattattatggatggacccccagctcagagccttggagtagaaccagtggataaagatgtcattcgtaaacctcctcgcaactggaaagacagcattttgactaaaaacttgatacttaaaatacttgtttcatcaataatcattgtttgtgggactttgtttgtcttctggcgtgagctacgagacaatgtgattacacctcgagacacaacaatgaccttcacatgctttgtgttttttgacatgttcaatgcactaagttccagatcccagaccaagtctgtgtttgagattggactctgcagtaatagaatgttttgctatgcagttcttggatccatcatgggacaattactagttatttactttcctccgcttcagaaggtttttcagactgagagcctaagcatactggatctgttgtttcttttgggtctcacctcatcagtgtgcatagtggcagaaattataaagaaggttgaaaggagcagggaaaagatccagaagcatgttagttcgacatcatcatcttttcttgaagtatga

SEQ ID NO: 4 - DNA, 3129 bp, Homo sapiens
  source 1..3129: mol_type = genomic DNA
atggagaacgcgcacaccaagacggtggaggaggtgctgggccacttcggcgtcaacgagagtacggggctgagcctggaacaggtcaagaagcttaaggagagatggggctccaacgagttaccggctgaagaaggaaaaaccttgctggaacttgtgattgagcagtttgaagacttgctagttaggattttattactggcagcatgtatatcttttgttttggcttggtttgaagaaggtgaagaaacaattacagcctttgtagaaccttttgtaattttactcatattagtagccaatgcaattgtgggtgtatggcaggaaagaaatgctgaaaatgccatcgaagcccttaaggaatatgagcctgaaatgggcaaagtgtatcgacaggacagaaagagtgtgcagcggattaaagctaaagacatagttcctggtgatattgtagaaattgctgttggtgacaaagttcctgctgatataaggttaacttccatcaaatctaccacactaagagttgaccagtcaattctcacaggtgaatctgtctctgtcatcaagcacactgatcccgtccctgacccacgagctgtcaaccaagataaaaagaacatgctgttttctggtacaaacattgctgctgggaaagctatgggagtggtggtagcaactggagttaacaccgaaattggcaagatccgggatgaaatggtggcaacagaacaggagagaacaccccttcagcaaaaactagatgaatttggggaacagctttccaaagtcatctcccttatttgcattgcagtctggatcataaatattgggcacttcaatgacccggttcatggagggtcctggatcagaggtgctatttactactttaaaattgcagtggccctggctgtagcagccattcctgaaggtctgcctgcagtcatcaccacctgcctggctcttggaactcgcagaatggcaaagaaaaatgccattgttcgaagcctcccgtctgtggaaacccttggttgtacttctgttatctgctcagacaagactggtacacttacaacaaaccagatgtcagtctgcaggatgttcattctggacagagtggaaggtgatacttgttcccttaatgagtttaccataactggatcaacttatgcacctattggagaagtgcataaagatgataaaccagtgaattgtcaccagtatgatggtctggtagaattagcaacaatttgtgctctttgtaatgactctgctttggattacaatgaggcaaagggtgtgtatgaaaaagttggagaagctacagagactgctctcacttgcctagtagagaagatgaatgtatttgataccgaattgaagggtctttctaaaatagaacgtgcaaatgcctgcaactcagtcattaaacagctgatgaaaaaggaattcactctagagttttcacgtgacagaaagtcaatgtcggtttactgtacaccaaataaaccaagcaggacatcaatgagcaagatgtttgtgaagggtgctcctgaaggtgtcattgacaggtgcacccacattcgagttggaagtactaaggttcctatgacctctggagtcaaacagaagatcatgtctgtcattcgagagtggggtagtggcagcgacacactgcgatgcctggccctggccactcatgacaacccactgagaagagaagaaatgcaccttgaggactctgccaactttattaaatatgagaccaatctgaccttcgttggctgcgtgggcatgctggatcctccgagaatcgaggtggcctcctccgtgaagctgtgccggcaagcaggcatccgggtcatcatgatcactggggacaacaagggcactgctgtggccatctgtcgccgcatcggcatcttcgggcaggatgaggacgtgacgtcaaaagctttcacaggccgggagtttgatgaactcaacccctccgcccagcgagacgcctgcctgaacgcccgctgttttgctcgagttgaaccctcccacaagtctaaaatcgtagaatttcttcagtcttttgatgagattacagctatgactggcgatggcgtgaacgatgctcctgctctgaagaaagccgagattggcattgctatgggctctggcactgcggtggctaaaaccgcctctgagatggtcctggcggatgacaacttctccaccattgtggctgccgttgaggaggggcgggcaatctacaacaacatgaaacagttcatccgctacctcatctcgtccaacgtcggggaagttgtctgtattttcctgacagcagcccttggatttcccgaggctttgattcctgttcagctgctctgggtcaatctggtgacagatggcctgcctgccactgcactggggttcaaccctcctgatctggacatcatgaataaacctccccggaacccaaaggaaccattgatcagcgggtggctctttttccgttacttggctattggctgttacgtcggcgctgctaccgtgggtgctgctgcatggtggttcattgctgctgacggtggtccaagagtgtccttctaccagctgagtcatttcctacagtgtaaagaggacaacccggactttgaaggcgtggattgtgcaatctttgaatccccatacccgatgacaatggcgctctctgttctagtaactatagaaatgtgtaacgccctcaacagcttgtccgaaaaccagtccttgctgaggatgcccccctgggagaacatctggctcgtgggctccatctgcctgtccatgtcactccacttcctgatcctctatgtcgaacccttgccactcatcttccagatcacaccgctgaacgtgacccagtggctgatggtgctgaaaatctccttgcccgtgattctcatggatgagacgctcaagtttgtggcccgcaactacctggaacctggtaaagagtgtgtgcagcctgccaccaaatcctgctcgttctcggcatgcaccgatgggatttcctggccgtttgtgctgctcataatgcccctggtgatctgggtctatagcacagacactaactttagcgatatgttctggtcttga

SEQ ID NO: 5 - DNA, 2760 bp, synthetic construct
  source 1..2760: mol_type = other DNA
atgaaggtggcccggttccagaagatccccaacggcgagaacgagacaatgatccccgtgctgaccagcaagaaggcctctgagctgcctgtgtctgaggtggccagtatcctgcaggccgatctgcagaacggactgaacaagtgcgaggtgtcccacagaagggccttccacggctggaacgagttcgacatcagcgaggatgagcccctgtggaagaagtacatcagccagttcaagaacccgctgatcatgctgctgctggcctctgccgtgatcagcgttctgatgcaccagttcgacgacgccgtgtctatcaccgtggccatcctgatcgtggtcacagtggccttcgtgcaagagtacagaagcgagaagtccctggaagaactgagcaagctggtgcctcctgagtgccactgtgtcagagagggcaagctggaacacaccctggccagagatctggtgcctggcgataccgtgtgtctgtccgtgggagatagagtgcccgccgacctgagactgtttgaggccgtggatctgagcatcgacgagagcagtctgaccggcgagacaaccccttgcagcaaagtgacagctcctcagcctgccgccaccaatggcgatctggcctccagatccaatatcgccttcatgggcaccctcgtcagatgcggaaaggccaagggcgttgtgatcggcacaggcgagaatagcgagttcggcgaggtgttcaagatgatgcaggctgaagaggcccctaagacacccctgcagaaatccatggacctgctgggcaagcagctgagcttctacagcttcggcatcatcggaatcatcatgctcgtcggctggctgctgggaaaagacatcctggaaatgttcaccatcagcgtgtccctggccgtggccgctattcctgaaggactgcctatcgtcgtgaccgtgacactggctctgggcgtgatgcggatggtcaagaaacgggccatcgtgaagaagctgcccatcgtcgagacactgggctgctgcaatgtgatctgcagcgacaagaccggcacactgaccaagaacgagatgacagtgacccacatcttcaccagcgacggcctgcatgctgaagtgacaggcgtgggctacaaccagtttggcgaagtgatcgtggacggcgacgtggtgcacggcttttacaatcctgccgtgtccagaatcgtggaagccggctgcgtgtgcaatgacgccgtgatccggaacaataccctgatgggaaagcctaccgaaggcgccctgattgccctggccatgaagatgggactcgatggcctgcagcaggactacatccggaaggccgagtatcccttcagcagcgagcagaaatggatggccgtgaagtgcgtgcacagaacccagcaggacagacccgagatctgctttatgaagggcgcctacgagcaagtgatcaagtactgcaccacctaccagagcaagggccagacactgaccctgacacagcagcagagagatgtgtatcagcaagagaaggcccggatgggctctgccggactgagagttcttgctctcgcctctggacctgagctgggccagctgacatttctgggactcgtgggaatcatcgaccctccaagaaccggcgtgaaagaagccgtgacaaccctgatcgccagcggcgtgtccattaagatgatcaccggcgacagccaagagacagccgtggctattgcctctagactgggcctgtacagcaagaccagccagtccgtgtctggggaagagatcgacgccatggatgtgcagcagctgtcccagatcgtgcctaaggtggccgtgttctaccgggcctctcctagacacaagatgaagatcatcaagagcctccagaaaaacggcagcgtggtggccatgactggcgacggtgttaatgatgccgtggctctgaaggccgccgatattggcgttgccatgggacagacaggcaccgacgtgtgtaaagaggccgctgacatgatcctggtggacgacgacttccagaccatcatgagcgccatcgaggaaggcaagggcatctacaacaacatcaagaacttcgtgcggttccagctgagcacctctatcgccgctctgacactgatcagcctggccacactgatgaacttccccaatcctctgaacgccatgcagatcctgtggatcaacatcatcatggacggccctccagctcagtccctgggagttgaacccgtggacaaggacgtgatcagaaagcctcctcggaactggaaggacagcatcctgacaaagaacctgatcctgaagattctggtgtccagcatcatcatcgtgtgcggcaccctgttcgtgttctggcgcgagctgagagacaacgtgatcacccctagggacaccaccatgaccttcacatgcttcgtgtttttcgacatgttcaacgccctgagcagccgcagccagaccaagtccgtgtttgagatcggactgtgcagcaaccggatgttctgctatgccgtgctgggcagcattatggggcagctgctcgtgatctacttcccaccactgcagaaggtgttccagaccgagagcctgtctatcctggatctgctgtttctgctgggcctgaccagctccgtgtgtatcgtggccgagatcattaagaaggtggaacggtcccgcgagaagatccagaaacacgtgtcctctaccagctccagcttcctggaagtgtga

SEQ ID NO: 6 - DNA, 3129 bp, synthetic construct
  source 1..3129: mol_type = other DNA
atggaaaacgcccacaccaagaccgtggaagaggtgctgggacacttcggcgtgaacgagagcacaggactgagcctggaacaagtgaagaaactgaaagagcgctggggcagcaacgagctgcctgccgaagagggaaagaccctgctggaactggtcatcgagcagttcgaggacctgctcgtgcggattctgctgctggccgcctgtatcagctttgtgctggcttggtttgaggaaggcgaggaaaccatcaccgccttcgtggaacccttcgtgatcctgctgatcctggtggccaatgccatcgtcggagtgtggcaagagagaaacgccgagaacgccatcgaggccctgaaagaatacgagcccgagatgggcaaagtgtaccggcaggacagaaagagcgtgcagcggatcaaggccaaggacatcgtgcctggcgacatcgtggaaatcgccgtgggagataaggtgcccgccgacatcagactgaccagcatcaagagcaccacactgagagtggaccagagcatcctgacaggcgagagcgtgtccgtgatcaagcacaccgatcctgtgcctgatcctcgggccgtgaaccaggacaagaaaaacatgctgttcagcggcaccaatatcgccgctggcaaggccatgggagttgtggttgctacaggcgtgaacaccgagatcggcaagatccgggatgagatggtggccacagagcaagagaggacccctctgcagcagaagctggacgagtttggcgagcagctgagcaaagtgatcagcctgatctgtatcgccgtgtggatcatcaacatcggccacttcaacgaccctgtgcacggcggctcttggattagaggcgccatctactacttcaagatcgctgtggccctggccgtggccgctattcctgaaggactgccagccgtgatcaccacctgtctggctctgggcacaagacggatggccaagaaaaatgccattgtgcggagcctgcctagcgtggaaacactgggctgtaccagcgtgatctgcagcgacaagacaggcaccctgaccaccaaccagatgagcgtgtgccggatgttcatcctggacagagtggaaggcgatacctgcagcctgaacgagttcacaatcaccggcagcacatacgcccctatcggcgaggtgcacaaggacgacaagcccgtgaattgccaccagtacgacggcctggttgagctggccacaatctgcgccctgtgcaatgatagcgccctggactacaatgaggccaagggcgtgtacgagaaagtgggcgaagccacagagacagccctgacctgtctcgtggaaaagatgaacgtgttcgacaccgagctgaagggcctgagcaagatcgaaagagccaacgcctgcaacagcgtcatcaagcagctgatgaagaaagagttcaccctcgagttcagccgcgaccggaagtccatgtccgtgtactgcacccctaacaagcccagcagaaccagcatgtccaagatgttcgtgaagggcgctcccgagggcgtgatcgatagatgcacacacatcagagtgggcagcaccaaggtgccaatgaccagcggcgtgaagcagaaaatcatgagcgtgatcagagagtggggcagcggcagcgataccctgagatgtttggctctggctacccacgacaaccctctgcggagagaagagatgcacctggaagatagcgccaacttcattaagtacgagacaaacctgaccttcgtgggctgcgtgggaatgctggaccctcctagaattgaggtggcctccagcgtgaagctgtgtagacaggccggaatccgcgtgatcatgattaccggcgacaacaagggaaccgccgtggccatctgtagacggatcggcatctttggccaggacgaggacgtgaccagcaaggcctttaccggcagagagttcgacgagctgaacccctccgctcagagagatgcctgtctgaacgccagatgcttcgcccgcgtggaacctagccacaagtccaagatcgtcgagttcctgcagagcttcgatgagatcaccgccatgactggcgacggcgttaacgatgcccctgctctgaagaaggccgagattggaatcgccatgggctctggaacagctgtggccaagacagccagcgaaatggtgctggccgacgacaacttcagcacaatcgtggccgcagtggaagaaggcagagccatctacaacaacatgaagcagttcatccgctacctgatcagcagcaacgtgggagaagtcgtgtgtatcttcctgaccgccgctctgggctttcccgaggctctgattcctgttcagctgctgtgggtcaacctggtcaccgatggactgcctgctacagccctgggcttcaaccctcctgacctggacatcatgaacaagcctccacggaaccccaaagagcccctgatctctggctggctgttcttcagatacctggccatcggctgctatgtgggagccgctacagttggagctgccgcctggtggtttattgccgctgatggcggaccccgggtgtccttttatcagctgagccacttcctgcagtgcaaagaggacaaccccgacttcgaaggcgtggactgcgccatcttcgagagcccctatcctatgacaatggccctgagcgtgctcgtgaccatcgagatgtgcaacgccctgaacagcctgagcgagaaccagagcctgctgagaatgcctccttgggagaacatctggctcgtggggagcatctgcctgtctatgagcctgcacttcctgatcctgtacgtggaaccactgcctctgatcttccagatcacccctctgaacgtgacccagtggctgatggtgctgaagatctccctgcctgtgatcctgatggacgagacactgaagttcgtggcccggaactacctggaacctggcaaagaatgtgtgcagcccgccaccaagtcctgcagctttagcgcctgtaccgacggcatcagctggccttttgtgctgctcatcatgcccctggtcatctgggtgtacagcaccgacaccaacttctccgacatgttctggtcctga

SEQ ID NO: 7 - DNA, 75 bp, synthetic construct
  source 1..75: mol_type = other DNA
agggccaagaggggcagcggcgagggcaggggcagcctgctgacctgcggcgacgtggaggagaaccccggcccc

SEQ ID NO: 8 - DNA, 66 bp, synthetic construct
  source 1..66: mol_type = other DNA
ggaagcggagctactaacttcagcctgctgaagcaggctggagacgtggaggagaaccctggacct

SEQ ID NO: 9 - DNA, 69 bp, synthetic construct
  source 1..69: mol_type = other DNA
ggaagcggacagtgtactaattatgctctcttgaaattggctggagatgttgagagcaaccctggacct

SEQ ID NO: 10 - DNA, 75 bp, synthetic construct
  source 1..75: mol_type = other DNA
ggaagcggagtgaaacagactttgaattttgaccttctcaagttggcgggagacgtggagtccaaccctggacct

SEQ ID NO: 11 - protein, 25 aa, synthetic construct
RAKRGSGEGRGSLLTCGDVEENPGP

SEQ ID NO: 12 - protein, 22 aa, synthetic construct
GSGATNFSLLKQAGDVEENPGP

SEQ ID NO: 13 - protein, 23 aa, synthetic construct
GSGQCTNYALLKLAGDVESNPGP

SEQ ID NO: 14 - protein, 25 aa, synthetic construct
GSGVKQTLNFDLLKLAGDVESNPGP

SEQ ID NO: 15 - protein, 15 aa, synthetic construct
GGGGSGGGGSGGGGS

SEQ ID NO: 16 - protein, 18 aa, synthetic construct
GGSSRSSSSGGGGSGGGG

SEQ ID NO: 17 - protein, 20 aa, synthetic construct
GGGGSGGGGSGGGGSGGGGS

SEQ ID NO: 18 - protein, 15 aa, synthetic construct
CGGGSGGGGSGGGGS

SEQ ID NO: 19 - protein, 15 aa, synthetic construct
SHGGHGGGGSGGGGS

SEQ ID NO: 20 - protein, 15 aa, synthetic construct
MGGMSGGGGSGGGGS

SEQ ID NO: 21 - protein, 15 aa, synthetic construct
YGGYSGGGGSGGGGS

SEQ ID NO: 22 - protein, 15 aa, synthetic construct
WGGYSGGGGSGGGGS

SEQ ID NO: 23 - protein, 12 aa, synthetic construct
SVSVGMKPSPRP

SEQ ID NO: 24 - protein, 12 aa, synthetic construct
VISNHAGSSRRL

SEQ ID NO: 25 - protein, 12 aa, synthetic construct
PWIPTPRPTFTG

SEQ ID NO: 26 - protein, 11 aa, synthetic construct
RGRGRGRGRGR

(19) United States
(12) Patent Application Publication
(10) Pub. No.: US 2026/0286394 A1
(43) Pub. Date: Sep. 24, 2026

(54) Compositions and Methods for the Treatment of ATPase Disorders or Diseases of the Skin

(71) Applicant: Krystal Biotech. Inc., Pittsburgh, PA (US)

(72) Inventors: Suma Krishnan, Pittsburgh, PA (US); Mary Jane Duermeyer, Gibsonia, PA (US); Bruce Chike Nmezi, Pittsburgh, PA (US); Trevor Parry, San Diego, CA (US)

(21) Appl. No.: 19/629,222

(22) Filed: Mar. 26, 2026

Related U.S. Application Data
(62) Division of application No. 19/377,489, filed on Nov. 3, 2025.
(60) Provisional application No. 63/715,105, filed on Nov. 1, 2024.

(51) Int. Cl. C12N 15/86; A61K 38/43; A61K 48/00; A61P 17/00

(52) U.S. Cl. CPC C12N 15/86; A61K 38/43; A61K 48/005; A61K 48/0075; A61P 17/00; C12N 2710/16643

(57) ABSTRACT

The present disclosure provides recombinant nucleic acids comprising one or more polynucleotides encoding an ATPase polypeptide (e.g., a calcium-transporting ATPase type 2C member 1 polypeptide or a sarcoplasmic/endoplasmic reticulum calcium ATPase 2 polypeptide); viruses comprising the recombinant nucleic acids; compositions and formulations comprising the recombinant nucleic acids and/or viruses; methods of their use (e.g., for treating an ATPase disorder or disease of the skin, for example, Hailey-Hailey disease or Darier disease); and articles of manufacture or kits thereof.

### CROSS REFERENCE TO RELATED APPLICATIONS

This application is a divisional of U.S. patent application Ser. No. 19/377,489, filed Nov. 3, 2025, which claims the priority benefit of U.S. Provisional Application No. 63/715,105, filed Nov. 1, 2024, the contents of which are incorporated herein by reference in their entirety.

### SUBMISSION OF SEQUENCE LISTING ON ASCII TEXT FILE

The Sequence Listing associated with this application is filed in electronic format via Patent Center and is hereby incorporated by reference into the specification in its entirety. The name of the file containing the Sequence Listing is 2504921.xml. The size of the file is 37,677 bytes, and the file was created on Oct. 29, 2025.

### FIELD OF THE INVENTION

The present disclosure relates, in part, to recombinant nucleic acids comprising one or more polynucleotides encoding an ATPase polypeptide (e.g., a calcium-transporting ATPase type 2C member 1 polypeptide or a sarcoplasmic/endoplasmic reticulum calcium ATPase 2 polypeptide), viruses comprising the same, pharmaceutical compositions and formulations thereof, and methods of their use (e.g., for treating an ATPase disorder or disease of the skin, for example, Hailey-Hailey disease or Darier disease).

### BACKGROUND

Hailey-Hailey disease (also known as familial benign chronic pemphigus) and Darier disease (also known as follicular dyskeratosis) are genodermatoses. Both Hailey-Hailey disease and Darier disease are characterized by impaired adhesion of epidermal keratinocytes.

Hailey-Hailey is characterized by mutations in the ATP2C1 gene which encodes a calcium pump termed Calcium-transporting ATPase type 2C member 1 (also referred to as “SPCA1” or “ATPase2C1”) resulting in decreased calcium concentration within the Golgi apparatus leading to impaired adhesion of epidermal keratinocytes. Individuals with Hailey-Hailey disease develop intertriginous papulovesicles and small blisters, which often evolve into erythematous plaques with erosions and painful fissures

Darier disease is characterized by mutations in the ATP2A2 gene which encodes a calcium pump termed Sarcoplasmic/endoplasmic reticulum calcium ATPase 2 (also referred to as “SERCA2” or “SR Ca²⁺-ATPase2”) resulting in impaired calcium homeostasis in keratinocytes and decreased cell-cell adhesion. Individuals with Darier disease are characterized by reddish brown, keratotic papules in seborrheic and intertriginous areas, which may coalesce into extensive lesions.

Individuals with Hailey-Hailey disease or Darier disease have significantly reduced quality of life due to, for example, itching, burning sensation, pain, and body malodor. Therapeutic options remain limited, and thus, there exists a need for more effective therapies.

All references cited herein, including patent applications, patent publications, non-patent literature, and NCBI/UniProtKB/Swiss-Prot Accession numbers are herein incorporated by reference in their entirety, as if each individual reference were specifically and individually indicated to be incorporated by reference.

### BRIEF SUMMARY

In order to meet these and other needs, provided herein are recombinant nucleic acids (e.g., recombinant herpes virus genomes) encoding one or more polypeptides (e.g., one or more ATPase polypeptides such as Calcium-transporting ATPase type 2C member 1 or Sarcoplasmic/endoplasmic reticulum calcium ATPase 2) for use in viruses (e.g., herpes viruses), pharmaceutical compositions and formulations, medicaments, and/or methods useful for treating a condition or disease in a subject in need thereof (e.g., a disease associated with a mutation in an underlying ATPase gene).

Accordingly, certain aspects of the present disclosure relate to a recombinant herpes virus genome comprising one or more polynucleotides encoding an ATPase polypeptide (e.g., a Calcium-transporting ATPase type 2C member 1 polypeptide or a Sarcoplasmic/endoplasmic reticulum calcium ATPase 2 polypeptide). In some embodiments, the recombinant herpes virus genome comprises two or more polynucleotides encoding an ATPase polypeptide. In some embodiments, the recombinant herpes virus genome is replication competent. In some embodiments, the recombinant herpes virus genome is replication defective. In some embodiments that may be combined with any of the preceding embodiments, the recombinant herpes virus genome comprises the one or more polynucleotides encoding the ATPase polypeptide within one or more viral gene loci. In some embodiments that may be combined with any of the preceding embodiments, the recombinant herpes virus genome is selected from a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, an Epstein-Barr virus genome, a recombinant Kaposi's sarcoma-associated herpesvirus genome, and any combinations or derivatives thereof.

In some embodiments that may be combined with any of the preceding embodiments, the recombinant herpes virus genome is a recombinant herpes simplex virus genome. In some embodiments, the recombinant herpes simplex virus genome is a recombinant type 1 herpes simplex virus (HSV-1) genome, a recombinant type 2 herpes simplex virus (HSV-2) genome, or any combinations or derivatives thereof. In some embodiments, the recombinant herpes simplex virus genome is a recombinant HSV-1 genome.

In some embodiments that may be combined with any of the preceding embodiments, the recombinant herpes simplex virus genome has been engineered to reduce or eliminate expression of one or more herpes simplex virus genes (e.g., one or more toxic herpes simplex virus genes). In some embodiments that may be combined with any of the preceding embodiments, the recombinant herpes simplex virus genome comprises an inactivating mutation. In some embodiments, the inactivating mutation is in a herpes simplex virus gene. In some embodiments, the inactivating mutation is a deletion of the coding sequence of the herpes simplex virus gene. In some embodiments, the herpes simplex virus gene is selected from Infected Cell Protein (ICP) 0 (one or both copies), ICP4 (one or both copies), ICP22, ICP27, ICP47, thymidine kinase (tk), Long Unique Region (UL) 41, and UL55. In some embodiments that may be combined with any of the preceding embodiments, the recombinant herpes simplex virus genome comprises an inactivating mutation in one or both copies of the ICP4 gene. In some embodiments that may be combined with any of the preceding embodiments, the recombinant herpes simplex virus genome comprises an inactivating mutation in the ICP22 gene. In some embodiments that may be combined with any of the preceding embodiments, the recombinant herpes simplex virus genome comprises an inactivating mutation in the UL41 gene. In some embodiments that may be combined with any of the preceding embodiments, the recombinant herpes simplex virus genome comprises an inactivating mutation in one or both copies of the ICPO gene. In some embodiments that may be combined with any of the preceding embodiments, the recombinant herpes simplex virus genome comprises an inactivating mutation in the ICP27 gene. In some embodiments that may be combined with any of the preceding embodiments, the recombinant herpes simplex virus genome comprises an inactivating mutation in the UL55 gene. In some embodiments that may be combined with any of the preceding embodiments, the recombinant herpes simplex virus genome does not comprise an inactivating mutation in the ICP47 gene. In some embodiments, the recombinant herpes simplex virus genome does not comprise an inactivating mutation in one or both copies of the ICP34.5 gene. In some embodiments that may be combined with any of the preceding embodiments, the recombinant herpes simplex virus genome does not comprise an inactivating mutation in the UL36 gene.

In some embodiments that may be combined with any of the preceding embodiments, the recombinant herpes simplex virus genome comprises the one or more polynucleotides encoding the ATPase polypeptide (e.g., a Calcium-transporting ATPase type 2C member 1 polypeptide or a Sarcoplasmic/endoplasmic reticulum calcium ATPase 2 polypeptide) within one or more viral gene loci. In some embodiments that may be combined with any of the preceding embodiments, the recombinant herpes simplex virus genome comprises the one or more polynucleotides encoding the ATPase polypeptide within one or both of the ICP4 viral gene loci. In some embodiments that may be combined with any of the preceding embodiments, the recombinant herpes simplex virus genome comprises the one or more polynucleotides encoding the ATPase polypeptide within the ICP22 viral gene locus. In some embodiments that may be combined with any of the preceding embodiments, the recombinant herpes simplex virus genome comprises the one or more polynucleotides encoding the ATPase polypeptide within the UL41 viral gene locus. In some embodiments that may be combined with any of the preceding embodiments, the recombinant herpes simplex virus genome comprises the one or more polynucleotides encoding the ATPase polypeptide within one or both of the ICPO viral gene loci. In some embodiments that may be combined with any of the preceding embodiments, the recombinant herpes simplex virus genome comprises the one or more polynucleotides encoding the ATPase polypeptide within the ICP27 viral gene locus. In some embodiments that may be combined with any of the preceding embodiments, the recombinant herpes simplex virus genome comprises the one or more polynucleotides encoding the ATPase polypeptide within the UL55 viral gene locus.

In some embodiments that may be combined with any of the preceding embodiments, the ATPase polypeptide (e.g., a Calcium-transporting ATPase type 2C member 1 polypeptide or a Sarcoplasmic/endoplasmic reticulum calcium ATPase 2 polypeptide) is a human ATPase polypeptide. In some embodiments that may be combined with any of the preceding embodiments, the ATPase polypeptide is a Calcium-transporting ATPase type 2C member 1 polypeptide. In some embodiments that may be combined with any of the preceding embodiments, the ATPase polypeptide is a human Calcium-transporting ATPase type 2C member 1 polypeptide. In some embodiments that may be combined with any of the preceding embodiments, the ATPase polypeptide is a Sarcoplasmic/endoplasmic reticulum calcium ATPase 2 polypeptide. In some embodiments that may be combined with any of the preceding embodiments, the ATPase polypeptide is a human Sarcoplasmic/endoplasmic reticulum calcium ATPase 2 polypeptide.

In some embodiments that may be combined with any of the preceding embodiments, the recombinant herpes virus genome has reduced cytotoxicity when introduced into a target cell as compared to a corresponding wild-type herpes virus genome. In some embodiments, the target cell is a human cell. In some embodiments, the target cell is a cell of the epidermis and/or dermis. In some embodiments, the target cell is a keratinocyte, a basal cell, a melanocyte, a squamous cell, a Langerhans cell, a Merkel cell, an/or a fibroblast.

Other aspects of the present disclosure relate to a herpes virus comprising any of the recombinant herpes virus genomes described herein. In some embodiments, the herpes virus is replication competent. In some embodiments, the herpes virus is replication defective. In some embodiments that may be combined with any of the preceding embodiments, the herpes virus is not oncolytic. In some embodiments that may be combined with any of the preceding embodiments, the herpes virus is a pseudotyped virus. In some embodiments that may be combined with any of the preceding embodiments, the herpes virus is not a pseudotyped virus. In some embodiments that may be combined with any of the preceding embodiments, the herpes virus is not a pseudotyped oncolytic virus. In some embodiments that may be combined with any of the preceding embodiments, the herpes virus has reduced cytotoxicity as compared to a corresponding wild-type herpes virus. In some embodiments that may be combined with any of the preceding embodiments, the herpes virus is selected from a herpes simplex virus, a varicella zoster virus, a human cytomegalovirus, a herpesvirus 6A, a herpesvirus 6B, a herpesvirus 7, an Epstein-Barr virus, a Kaposi's sarcoma-associated herpesvirus, and any combinations or derivatives thereof. In some embodiments that may be combined with any of the preceding embodiments, the herpes virus is a herpes simplex virus. In some embodiments, the herpes simplex virus is not oncolytic. In some embodiments, the herpes simplex virus is an HSV-1, an HSV-2, or any combinations or derivatives thereof. In some embodiments, the herpes simplex virus is an HSV-1. In some embodiments, the HSV-1 is not oncolytic.

Other aspects of the present disclosure relate to a pharmaceutical composition comprising any of the recombinant herpes virus genomes and/or any of the recombinant herpes viruses described herein and a pharmaceutically acceptable carrier or excipient. In some embodiments that may be combined with any of the preceding embodiments, the pharmaceutical composition is suitable for topical, transdermal, subcutaneous, intradermal, oral, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, injection, or epicutaneous administration. In some embodiments that may be combined with any of the preceding embodiments, the pharmaceutical composition comprises a phosphate buffer. In some embodiments that may be combined with any of the preceding embodiments, the pharmaceutical composition comprises glycerol. In some embodiments that may be combined with any of the preceding embodiments, the pharmaceutical composition comprises a lipid carrier. In some embodiments that may be combined with any of the preceding embodiments, the pharmaceutical composition comprises a nanoparticle carrier.

Other aspects of the present disclosure relate to the use of any of the recombinant nucleic acids (e.g., recombinant herpes virus genomes), recombinant viruses (e.g., recombinant herpes viruses), and/or pharmaceutical compositions described herein as a medicament.

Other aspects of the present disclosure relate to the use of any of the recombinant nucleic acids (e.g., recombinant herpes virus genomes), recombinant viruses (e.g., recombinant herpes viruses), and/or pharmaceutical compositions described herein in a therapy.

Other aspects of the present disclosure relate to the use of any of the recombinant nucleic acids (e.g., recombinant herpes virus genomes), recombinant viruses (e.g., recombinant herpes viruses), and/or pharmaceutical compositions described herein in the preparation of a medicament for treating an ATPase disorder or disease (e.g., a disease or disorder associated with a mutation in an underlying ATPase gene). In some embodiments, the ATPase disorder or disease is autosomal dominant. In some embodiments, the ATPase disorder or disease is a haploinsufficiency. In some embodiments, the ATPase disorder or disease is a haploinsufficiency in one or more cells of the epidermis and/or dermis (e.g., a haploinsufficiency in a keratinocyte). In some embodiments, the ATPase disorder or disease is an autosomal dominant genodermatosis. In some embodiments, the ATPase disorder or disease is Haily-Hailey disease. In some embodiments, the ATPase disorder or disease is Darier disease.

Other aspects of the present disclosure relate to a method of expressing, enhancing, increasing, augmenting, and/or supplementing the levels of an ATPase polypeptide (e.g., a Calcium-transporting ATPase type 2C member 1 polypeptide or a Sarcoplasmic/endoplasmic reticulum calcium ATPase 2 polypeptide) in one or more cells of a subject comprising administering to the subject an effective amount of any of the recombinant herpes viruses and/or pharmaceutical compositions described herein. In some embodiments, the one or more cells are one or more cells of the epidermis and/or dermis. In some embodiments, the one or more cells are one or more keratinocytes, basal cells, melanocytes, squamous cells, Langerhans cells, Merkel cells, or fibroblasts. In some embodiments that may be combined with any of the preceding embodiments, the subject is a human. In some embodiments that may be combined with any of the preceding embodiments, the herpes virus or pharmaceutical composition is administered via injection, topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, intraarticularly, periarticularly, locally, or via inhalation to the subject. In some embodiments, the herpes virus or pharmaceutical composition is administered topically.

Other aspects of the present disclosure relate to a method of providing prophylactic, palliative, or therapeutic relief of one or more signs or symptoms of an ATPase disorder or disease in a subject in need thereof comprising administering to the subject an effective amount of any of the recombinant herpes viruses and/or pharmaceutical compositions described herein. In some embodiments, the ATPase disorder or disease is Hailey-Hailey disease. In some embodiments, the ATPase disorder or disease is Darier disease. In some embodiments that may be combined with any of the preceding embodiments, the subject is a human. In some embodiments that may be combined with any of the preceding embodiments, the herpes virus or pharmaceutical composition is administered via injection, topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, intraarticularly, periarticularly, locally, or via inhalation to the subject.

Other aspects of the present disclosure relate to any of the recombinant herpes viruses and/or pharmaceutical compositions described herein for use in the treatment of an ATPase disorder or disease in a subject in need thereof. In some embodiments, the ATPase disorder or disease is Hailey-Hailey disease. In some embodiments, the ATPase disorder or disease is Darier disease. In some embodiments, the subject is a human. In some embodiments that may be combined with any of the preceding embodiments, the herpes virus and/or pharmaceutical composition is administered via injection, topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, intraarticularly, periarticularly, locally, or via inhalation to the subject.

Other aspects of the present disclosure relate to a method of treating an ATPase disorder or disease in a subject in need thereof comprising administering to the subject an effective amount of any of the recombinant herpes viruses and/or pharmaceutical compositions described herein. In some embodiments, the ATPase disorder or disease is Hailey-Hailey disease. In some embodiments, the ATPase disorder or disease is Darier disease. In some embodiments, the subject is a human. In some embodiments that may be combined with any of the preceding embodiments, the herpes virus and/or pharmaceutical composition is administered via injection, topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, intraarticularly, periarticularly, locally, or via inhalation to the subject.

Other aspects of the present disclosure relate to a method of delivering an ATPase polypeptide (e.g., a Calcium-transporting ATPase type 2C member 1 polypeptide or a Sarcoplasmic/endoplasmic reticulum calcium ATPase 2 polypeptide) to one or more cells of the epidermis and/or dermis of a subject comprising administering to the subject a pharmaceutical composition comprising (a) a herpes virus comprising a recombinant herpes virus genome, wherein the recombinant herpes virus genome comprises one or more polynucleotides encoding the ATPase polypeptide (e.g., a Calcium-transporting ATPase type 2C member 1 polypeptide or a Sarcoplasmic/endoplasmic reticulum calcium ATPase 2 polypeptide), and (b) a pharmaceutically acceptable carrier. In some embodiments, the subject suffers from an ATPase disorder or disease (e.g., Hailey-Hailey disease or Darier disease) affecting the epidermis and/or dermis of the subject.

In some embodiments that may be combined with any of the preceding embodiments, the herpes virus is a pseudotyped virus. In some embodiments that may be combined with any of the preceding embodiments, the herpes virus is not a pseudotyped virus. In some embodiments that may be combined with any of the preceding embodiments, the herpes virus is not a pseudotyped oncolytic virus.

Other aspects of the present disclosure relate to an article of manufacture or kit comprising any of the recombinant nucleic acids, viruses, medicaments, and/or pharmaceutical compositions or formulations described herein and instructions for administration thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1I show schematics of wild-type and modified herpes simplex virus genomes. FIG. 1A shows a wild-type herpes simplex virus genome. FIG. 1B shows a modified herpes simplex virus genome comprising deletions of the coding sequence of ICP4 (both copies), with an expression cassette containing a nucleic acid encoding an ATPase polypeptide integrated at each of the ICP4 loci. FIG. 1C shows a modified herpes simplex virus genome comprising deletions of the coding sequences of ICP4 (both copies) and UL41, with an expression cassette containing a nucleic acid encoding an ATPase polypeptide integrated at each of the ICP4 loci. FIG. 1D shows a modified herpes simplex virus genome comprising deletions of the coding sequences of ICP4 (both copies) and UL41, with an expression cassette containing a nucleic acid encoding an ATPase polypeptide integrated at the UL41 locus. FIG. 1E shows a modified herpes simplex virus genome comprising deletions of the coding sequences of ICP4 (both copies) and ICP22, with an expression cassette containing a nucleic acid encoding an ATPase polypeptide integrated at each of the ICP4 loci. FIG. 1F shows a modified herpes simplex virus genome comprising deletions of the coding sequences of ICP4 (both copies) and ICP22, with an expression cassette containing a nucleic acid encoding an ATPase polypeptide integrated at the ICP22 locus. FIG. 1G shows a modified herpes simplex virus genome comprising deletions of the coding sequences of ICP4 (both copies), UL41, and ICP22, with an expression cassette containing a nucleic acid encoding an ATPase polypeptide integrated at each of the ICP4 loci. FIG. 1H shows a modified herpes simplex virus genome comprising deletions of the coding sequences of ICP4 (both copies), UL41, and ICP22, with an expression cassette containing a nucleic acid encoding an ATPase polypeptide integrated at the UL41 locus. FIG. 1I shows a modified herpes simplex virus genome comprising deletions of the coding sequences of ICP4 (both copies), UL41, and ICP22, with an expression cassette containing a nucleic acid encoding an ATPase polypeptide integrated at the ICP22 locus.

FIG. 2 depicts HSV-ATP2C1 transduction (FIG. 2A) and ATP2C1 transgene expression (FIG. 2B) in transduced HaCaT cells. Data are presented as the average of technical duplicates+standard error of the mean (SEM). *=samples were below the limit of quantification.

FIG. 3 shows dose-dependent (multiplicity of infection (MOI) of 0 (mock), 0.3, 1, 3, or 5) ATP2C1 protein expression (FIG. 3A; western blot) and protein abundance (FIG. 3B; quantification of ATP2C1 protein intensity, normalized against total protein) in HSV-ATP2C1 transduced HaCaT cells.

FIG. 4 depicts HSV-ATP2C1 encoded ATP2C1 protein localizes in the Golgi apparatus in transduced HaCaT cells. ATP2C1 (green) was co-stained with DAPI (nuclei marker; blue), and/or GOLGA4 (Golgin A4 (GOLGA4); a Golgi apparatus marker; red).

FIG. 5 shows live/dead flow cytometry gating using viability fixable dye in HaCaT cells. FIG. 5A: Representative histograms of live (left panel) and dead (right panel) control cell staining using Viobility 405/452 Fixable Dye in HaCaT cells. Red lines across the histograms depict fluorescence intensity thresholds used to quantify cell viability in transduced cells. FIG. 5B: Cell viability in mock and transduced cells 24 hours (top panel) and 48 hours (bottom panel) after treatment.

FIG. 6A depicts endogenous ATP2C1 expression in HaCaT cells 24 hours post-siRNA treatment. FIG. 6B depicts codon-optimized ATP2C1 expression in HaCaT cells 24 hours post siRNA and/or HSV-ATP2C1 treatment. Data are presented as the average of technical duplicates +SEM.

FIG. 7A shows immunofluorescence representative images of F-Actin (phalloidin; red) and DAPI (nuclei marker; blue) staining in siRNA and/or HSV-ATP2C1 (MOI of 1) 24-hours post treatment in HaCaT cells. All images were taken with 40× objective magnification. Scale bar=25 μm. FIG. 7B shows the immunofluorescence quantification of F-Actin in siRNA and/or HSV-ATP2C1 (MOI of 1) 24-hours post treatment in HaCaT cells. Data are presented as the mean red fluorescence intensity of cell clusters+SEM. AU=arbitrary units; *=p<0.05; ***=p<0.0001, calculated using one-way analysis of variance (ANOVA) and Tukey's multiple comparisons test.

FIG. 8 depicts vector transduction and transgene expression in mouse skin 24 hours post-HSV-ATP2C1 topical administration. Data are presented as the average+SEM of n=3 animals per group. *=samples were below the limit of quantification.

FIG. 9 shows immunofluorescence images of human ATP2C1 protein localization in mouse skin 24 hours after topical administration of HSV-ATP2C1. White arrows indicate areas in the epidermis with strong ATP2C1 (red) signal. Nuclei were visualized with DAPI staining (blue). All images were taken with 20× objective magnification.

FIG. 10 depicts representative time course (24 hours to 7 days after topical HSV-ATP2C1 treatment) progression images of one mouse from the vehicle-control group (top) and one mouse from the HSV-ATP2C1 treated (bottom) group.

FIG. 11 shows histological analysis of vehicle control or HSV-ATP2C1-treated skin sections after 24 hours and 7 days following treatment. Images are representative of hematoxylin and eosin (H&E)-stained skin tissue biopsies from each time point. All images were taken with 20× objective magnification.

FIG. 12 depicts vector transduction and transgene expression kinetics in mouse skin after HSV-ATP2C1 administration. Data are presented as the average+SEM of n=3 animals per group. *=samples were below the limit of quantification.

FIG. 13 shows representative images of hematoxylin and eosin (H&E) histological analysis of vehicle or HSV-ATP2C1 treated skin sections from 4 hours to 7 days. All images were taken with 20× objective magnification. Scale bar=50 μm.

FIG. 14 depicts HSV-ATP2A2 transduction (FIG. 14A) and ATP2A2 transgene expression (FIG. 14B) in transduced HaCaT cells. Data are presented as the average of technical duplicates+standard error of the mean (SEM). *=samples were below the limit of quantification.

FIG. 15 shows dose-dependent (multiplicity of infection (MOI) of 0 (mock), 0.3, 1, or 3) ATP2A2 protein expression (FIG. 15A; western blot) and protein abundance (FIG. 15B; quantification of ATP2A2 protein intensity, normalized against total protein) in HSV-ATP2A2 transduced HaCaT cells.

FIG. 16 depicts HSV-ATP2A2 encoded ATP2A2 protein localizes in the endoplasmic reticulum in transduced HaCaT cells. ATP2A2 (red) was co-stained with DAPI (nuclei marker; blue), and Calnexin (an endoplasmic reticulum marker; green).

FIG. 17 shows cell viability via a Mosmann's Tetrazolium Toxicity assay in mock and transduced cells 48 hours after treatment.

FIG. 18 depicts vector transduction and transgene expression in mouse skin 24 hours post-HSV-ATP2A2 topical administration. Data are presented as the average+SEM of n=3 animals per group. *=samples were below the limit of quantification.

FIG. 19 shows immunofluorescence images of human ATP2A2 protein localization in mouse skin following topical administration of HSV-ATP2A2. ATP2A2 (red) was co-stained with DAPI (nuclei marker; blue). All images were taken with 20× objective magnification.

### DETAILED DESCRIPTION

The following description sets forth exemplary methods, parameters, and the like. It should be recognized, however, that such a description is not intended as a limitation on the scope of the present disclosure but is instead provided as a description of exemplary embodiments.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### II. Definitions

Before describing the present disclosure in detail, it is to be understood that the present disclosure is not limited to particular compositions or biological systems, which can, of course, vary. It is also to be understood that the terminology used herein is for the purpose of describing particular embodiments only and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

As used herein, the term “and/or” may include any and all combinations of one or more of the associated listed items. For example, the term “a and/or b” may refer to “a alone”, “b alone”, “a or b”, or “a and b”; the term “a, b, and/or c” may refer to “a alone”, “b alone”, “c alone”, “a or b”, “a or c”, “b or c”, “a, b, or c”, “a and b”, “a and c”, “b and c”, or “a, b, and c”; etc.

As used herein, the term “about” refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising”, “consisting”, and “consisting essentially of” aspects and embodiments.

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

As used herein, a nucleic acid is “operatively linked” or “operably linked” when it is placed into a functional relationship with another nucleic acid sequence. For example, a promoter or enhancer is operably linked to a coding sequence if it affects the transcription of the sequence, or a ribosome binding site is operably linked to a coding sequence if it is positioned so as to facilitate translation. Generally, “operatively linked” or “operably linked” means that the DNA or RNA sequences being linked are contiguous.

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of effecting expression of such nucleic acids. Thus, an expression vector may refer to a DNA or RNA construct, such as a plasmid, a phage, recombinant virus, or other vector that, upon introduction into an appropriate host cell, results in expression of the nucleic acids. Appropriate expression vectors are well known to those of skill in the art and include those that are replicable in eukaryotic cells and those that remain episomal or those which integrate into the host cell genome.

As used herein, an “open reading frame” or “ORF” refers to a continuous stretch of nucleic acids, either DNA or RNA, that encode a protein or polypeptide. Typically, the nucleic acids comprise a translation start signal or initiation codon, such as ATG or AUG, and a termination codon.

As used herein, an “untranslated region” or “UTR” refers to untranslated nucleic acids at the 5′ and/or 3′ ends of an open reading frame. The inclusion of one or more UTRs in a polynucleotide may affect post-transcriptional regulation, mRNA stability, and/or translation of the polynucleotide.

As used herein, the term “transgene” refers to a polynucleotide that is capable of being transcribed into RNA and translated and/or expressed under appropriate conditions after being introduced into a cell. In some aspects, it confers a desired property to a cell into which it was introduced, or otherwise leads to a desired therapeutic or diagnostic outcome.

As used herein, the terms “polypeptide,” “protein,” and “peptide” are used interchangeably and may refer to a polymer of two or more amino acids.

As used herein, a “subject”, “host”, or an “individual” refers to any animal classified as a mammal, including humans, domestic and farm animals, and zoo, sports, or pet animals, such as dogs, horses, cats, cows, as well as animals used in research, such as mice, rats, hamsters, rabbits, and non-human primates, etc. In some embodiments, the mammal is human.

As used herein, the terms “pharmaceutical formulation” or “pharmaceutical composition” refer to a preparation which is in such a form as to permit the biological activity of the active ingredient(s) to be effective, and which contains no additional components which are unacceptably toxic to a subject to which the composition or formulation would be administered. “Pharmaceutically acceptable” excipients (e.g., vehicles, additives) are those which can reasonably be administered to a subject mammal to provide an effective dose of the active ingredient(s) employed.

As used herein, an “effective amount” is at least the minimum amount required to affect a measurable improvement or prevention of one or more symptoms of a particular disorder. An “effective amount” may vary according to factors such as the disease state, age, sex, and weight of the patient. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications used to treat symptoms of the disease, delaying the progression of the disease, and/or prolonging survival. An effective amount can be administered in one or more administrations. For purposes of the present disclosure, an effective amount of a recombinant nucleic acid, virus, and/or pharmaceutical composition is an amount sufficient to accomplish prophylactic or therapeutic treatment either directly or indirectly. As is understood in the clinical context, an effective amount of a recombinant nucleic acid, virus, and/or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “treatment” refers to clinical intervention designed to alter the natural course of the individual or cell being treated during the course of clinical pathology. Desirable effects of treatment include decreasing the rate of disease/disorder/defect progression, ameliorating, or palliating the disease/disorder/defect state, and remission or improved prognosis.

As used herein, the term “delaying progression of” a disease/disorder/defect refers to deferring, hindering, slowing, retarding, stabilizing, and/or postponing development of the disease/disorder/defect. This delay can be of varying lengths or time, depending on the history of the disease/disorder/defect and/or the individual being treated. As is evident to one of ordinary skill in the art, a sufficient or significant delay can, in effect, encompass prevention, in that the individual does not develop the disease.

### III. Recombinant Nucleic Acids

Certain aspects of the present disclosure relate to recombinant nucleic acids (e.g., isolated recombinant nucleic acids) comprising one or more (e.g., one or more, two or more, three or more, four or more, five or more, ten or more, etc.) polynucleotides encoding an ATPase polypeptide. In some embodiments, the recombinant nucleic acid comprises one polynucleotide encoding an ATPase polypeptide. In some embodiments, the recombinant nucleic acid comprises two polynucleotides encoding an ATPase polypeptide. In some embodiments, the recombinant nucleic acid comprises three or more polynucleotides encoding an ATPase polypeptide. In some embodiments, the recombinant nucleic acid comprises one or more polynucleotides encoding two or more ATPase polypeptides. In some embodiments, the recombinant nucleic acid comprises two or more polynucleotides encoding two or more ATPase polypeptides. In some embodiments, the two or more ATPase polypeptides are identical. In some embodiments, the two or more ATPase polypeptides are different.

In some embodiments, the present disclosure relates to recombinant nucleic acids comprising a polynucleotide encoding a chimeric polypeptide comprising: a first ATPase polypeptide, a linker polypeptide, and a second ATPase polypeptide. In some embodiments, the first and second ATPase polypeptides are the same. In some embodiments, the first and second ATPase polypeptides are different. In some embodiments, the linker polypeptide is a cleavable linker polypeptide. In some embodiments, the linker polypeptide is a non-cleavable linker polypeptide.

In some embodiments, the recombinant nucleic acid is a vector. In some embodiments, the recombinant nucleic acid is a viral vector. In some embodiments, the recombinant nucleic acid is a herpes viral vector. In some embodiments, the recombinant nucleic acid is a herpes simplex virus amplicon. In some embodiments, the recombinant nucleic acid is a recombinant herpes virus genome. In some embodiments, the recombinant herpes virus genome is a recombinant herpes simplex virus genome. In some embodiments, the recombinant herpes simplex virus genome is a recombinant herpes simplex virus type 1 (HSV-1) genome.

### Polynucleotides Encoding ATPase Polypeptides

In some embodiments, the present disclosure relates to recombinant nucleic acids comprising one or more polynucleotides encoding one or more ATPase polypeptides (e.g., one or more human ATPase polypeptides). Any suitable ATPase polypeptide described herein or known in the art may be encoded by a polynucleotide of the present disclosure, including, for example, human ATPase polypeptides, such as human Calcium-transporting ATPase type 2C member 1 polypeptide or human Sarcoplasmic/endoplasmic reticulum calcium ATPase 2 polypeptide.

In some embodiments, a polynucleotide of the present disclosure comprises the wild-type coding sequence of any ATPase gene described herein or known in the art (including any isoform thereof). In some embodiments, a polynucleotide of the present disclosure comprises a codon-optimized variant of the wild-type coding sequence of any ATPase gene described herein or known in the art. In some embodiments, use of a codon-optimized variant of the coding sequence of a gene increases stability and/or yield of heterologous expression (RNA and/or protein) of the encoded polypeptide in a target cell, as compared to the stability and/or yield of heterologous expression of a corresponding, non-codon-optimized, wild-type sequence. Any suitable method known in the art for performing codon optimization of a sequence for expression in one or more target cells (e.g., one or more human cells) may be used, including, for example, by the methods described by Fath et al. (PLOS One. 2011 Mar. 3; 6 (3): e17596).

In some embodiments, the present disclosure relates to a recombinant nucleic acid comprising one or more polynucleotides comprising the coding sequence of a human ATPase gene. Any suitable human ATPase gene (including any isoform thereof) known in the art may be encoded by a polynucleotide of the present disclosure, including, for example, an ATP2C1 gene (see e.g., NCBI Gene ID: 27032; SEQ ID NO: 3), an ATP2A2 gene (see e.g., NCBI Gene ID: 488; SEQ ID NO: 4), etc. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the human ATPase genes (and/or coding sequences thereof) described herein or known in the art. In some embodiments, the ATPase gene is a human ATP2C1 gene or a human ATP2A2 gene.

In some embodiments, a polynucleotide of the present disclosure encodes a Calcium-transporting ATPase type 2C member 1 polypeptide. In some embodiments, the Calcium-transporting ATPase type 2C member 1 polypeptide is a human Calcium-transporting ATPase type 2C member 1polypeptide (see e.g., UniProt accession number: P98194). In some embodiments, the polynucleotide comprises the coding sequence of a wild-type ATP2C1 gene (see e.g., NCBI Gene ID: 27032, SEQ ID NO: 3), or a codon-optimized variant thereof (see e.g., SEQ ID NO: 5). In some embodiments, a polynucleotide encoding a Calcium-transporting ATPase type 2C member 1 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 1. In some embodiments, a polynucleotide encoding a Calcium-transporting ATPase type 2C member 1 polypeptide is a polynucleotide that encodes a polypeptide comprising the amino acid sequence of SEQ ID NO: 1.

In some embodiments, a polynucleotide encoding a Calcium-transporting ATPase type 2C member 1 polypeptide is a polynucleotide that encodes an N-terminal truncation, a C-terminal truncation, or a fragment of the amino acid sequence of SEQ ID NO: 1. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, but fewer than 919, consecutive amino acids of SEQ ID NO: 1.

In some embodiments, a polynucleotide of the present disclosure encodes a Sarcoplasmic/endoplasmic reticulum calcium ATPase 2 polypeptide. In some embodiments, the Sarcoplasmic/endoplasmic reticulum calcium ATPase 2 polypeptide is a human Sarcoplasmic/endoplasmic reticulum calcium ATPase 2 polypeptide (see e.g., UniProt accession number: P16615). In some embodiments, the polynucleotide comprises the coding sequence of a wild-type ATP2A2 gene (see e.g., NCBI Gene ID: 488, SEQ ID NO: 4), or a codon-optimized variant thereof (see e.g., SEQ ID NO: 6). In some embodiments, a polynucleotide encoding a Sarcoplasmic/endoplasmic reticulum calcium ATPase 2 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 2. In some embodiments, a polynucleotide encoding a Sarcoplasmic/endoplasmic reticulum calcium ATPase 2 polypeptide is a polynucleotide that encodes a polypeptide comprising the amino acid sequence of SEQ ID NO: 2.

In some embodiments, a polynucleotide encoding a Sarcoplasmic/endoplasmic reticulum calcium ATPase 2 polypeptide is a polynucleotide that encodes an N-terminal truncation, a C-terminal truncation, or a fragment of the amino acid sequence of SEQ ID NO: 2. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1,000 but fewer than 1,042, consecutive amino acids of SEQ ID NO: 2.

In some embodiments, a polynucleotide of the present disclosure encodes any one or more of a Calcium-transporting ATPase type 2C member 1 peptide, a Sarcoplasmic/endoplasmic reticulum calcium ATPase 2 peptide, and/or any chimeric polypeptides thereof, in any suitable combination.

A polynucleotide of the present disclosure encoding a polypeptide (e.g., an ATPase polypeptide) may further encode additional coding and non-coding sequences. Examples of additional coding and non-coding sequences may include, but are not limited to, sequences encoding additional polypeptide tags (e.g., encoded in-frame with the polypeptide in order to produce a fusion protein), introns (e.g., native, modified, or heterologous introns), 5′ and/or 3′ UTRs (e.g., native, modified, or heterologous 5′ and/or 3′ UTRs), and the like. Examples of suitable polypeptide tags may include, but are not limited, to any combination of purification tags, such as his-tags, flag-tags, maltose binding protein and glutathione-S-transferase tags, detection tags, such as tags that may be detected photometrically (e.g., green fluorescent protein, red fluorescent protein, etc.) and tags that have a detectable enzymatic activity (e.g., alkaline phosphatase, etc.), tags containing secretory sequences, signal sequences, leader sequences, and/or stabilizing sequences, protease cleavage sites (e.g., furin cleavage sites, TEV cleavage sites, Thrombin cleavage sites, etc.), and the like. In some embodiments, the 5′ and/or 3′UTRs increase the stability, localization, and/or translational efficiency of the polynucleotides. In some embodiments, the 5′ and/or 3′UTRs improve the level and/or duration of protein expression. In some embodiments, the 5′ and/or 3′UTRs include elements (e.g., one or more miRNA binding sites, etc.) that may block or reduce off-target expression (e.g., inhibiting expression in specific cell types (e.g., neuronal cells), at specific times in the cell cycle, at specific developmental stages, etc.). In some embodiments, the 5′ and/or 3′UTRs include elements (e.g., one or more miRNA binding sites, etc.) that may enhance expression of the encoded polypeptide in specific cell types.

In some embodiments, a polynucleotide of the present disclosure encoding a polypeptide (e.g., an ATPase polypeptide) is operably linked to one or more (e.g., one or more, two or more, three or more, four or more, five or more, ten or more, etc.) regulatory sequences. The term “regulatory sequence” may include enhancers, insulators, promoters, and other expression control elements (e.g., polyadenylation signals). Any suitable enhancer(s) known in the art may be used, including, for example, enhancer sequences from mammalian genes (such as globin, elastase, albumin, α-fetoprotein, insulin and the like), enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like), and any combinations thereof. Any suitable insulator(s) known in the art may be used, including, for example, HSV chromatin boundary (CTRL/CTCF-binding/insulator) elements CTRL1 and/or CTRL2, chicken hypersensitive site 4 insulator (cHS4), human HNRPA2B1-CBX3 ubiquitous chromatin opening element (UCOE), the scaffold/matrix attachment region (S/MAR) from the human interferon beta gene (IFNB1), and any combinations thereof. Any suitable promoter (e.g., suitable for transcription in mammalian host cells) known in the art may be used, including, for example, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), promoters from heterologous mammalian genes (such as the actin promoter (e.g., the β-actin promoter), a ubiquitin promoter (e.g., a ubiquitin C (UbC) promoter), a phosphoglycerate kinase (PGK) promoter, an immunoglobulin promoter, from heat-shock promoters, and the like), promoters from homologous mammalian genes, synthetic promoters (such as the CAG promoter), and any combinations thereof, provided such promoters are compatible with the host cells. Regulatory sequences may include those which direct constitutive expression of a nucleic acid, as well as tissue-specific regulatory and/or inducible or repressible sequences.

In some embodiments, a polynucleotide of the present disclosure is operably linked to one or more heterologous promoters. In some embodiments, the one or more heterologous promoters are one or more of constitutive promoters, tissue-specific promoters, temporal promoters, spatial promoters, inducible promoters, and repressible promoters. In some embodiments, the one or more heterologous promoters are one or more of the human cytomegalovirus (HCMV) immediate early promoter, the human elongation factor-1 (EF1) promoter, the human β-actin promoter, the human UbC promoter, the human PGK promoter, the synthetic CAGG promoter, and any combinations thereof. In some embodiments, a polynucleotide of the present disclosure encoding a polypeptide (e.g., an ATPase polypeptide) is operably linked to an HCMV promoter.

In some embodiments, a polynucleotide of the present disclosure encoding a polypeptide (e.g., an ATPase polypeptide, such as Calcium-transporting ATPase type 2C member 1 and/or Sarcoplasmic/endoplasmic reticulum calcium ATPase 2) expresses the polypeptide when the polynucleotide is delivered into one or more target cells of a subject (e.g., one or more cells of the epidermis and/or dermis of the subject). In some embodiments, expression of the polypeptide (e.g., an ATPase polypeptide, such as Calcium-transporting ATPase type 2C member 1 and/or Sarcoplasmic/endoplasmic reticulum calcium ATPase 2) enhances, increases, augments, and/or supplements the levels, function, and/or activity of the polypeptide in one or more target cells of a subject (e.g., as compared to prior to expression of the polypeptide, as compared to levels of the endogenous polypeptide expressed in the cell, etc.). In some embodiments, expression of the polypeptide (e.g., an ATPase polypeptide, such as Calcium-transporting ATPase type 2C member 1 and/or Sarcoplasmic/endoplasmic reticulum calcium ATPase 2) provides prophylactic, palliative, or therapeutic relief of one or more signs or symptoms of an ATPase disorder or disease (e.g., Hailey-Hailey disease and/or Darier disease) in a subject (e.g., as compared to prior to expression of the polypeptide).

In some embodiments, a polynucleotide of the present disclosure does not comprise the coding sequence of (e.g., a transgene encoding) a Collagen alpha-1 (VII) chain polypeptide (COL7). In some embodiments, a polynucleotide of the present disclosure does not comprise the coding sequence of (e.g., a transgene encoding) a Lysyl hydroxylase 3 polypeptide (LH3). In some embodiments, a polynucleotide of the present disclosure does not comprise the coding sequence of (e.g., a transgene encoding) a Keratin type I cytoskeletal 17 polypeptide (KRT17). In some embodiments, a polynucleotide of the present disclosure does not comprise the coding sequence of (e.g., a transgene encoding) a transglutaminase (TGM) polypeptide (e.g., a human transglutaminase polypeptide such as a human TGM1 polypeptide and/or a human TGM5 polypeptide). In some embodiments, a polynucleotide of the present disclosure does not comprise the coding sequence of (e.g., a transgene encoding) a cosmetic protein (e.g., collagen proteins, fibronectins, elastins, lumicans, vitronectins/vitronectin receptors, laminins, neuromodulators, fibrillins, additional dermal extracellular matrix proteins, etc.). In some embodiments, a polynucleotide of the present disclosure does not comprise the coding sequence of (e.g., a transgene encoding) an antibody (e.g., a full-length antibody, an antibody fragment, etc.). In some embodiments, a polynucleotide of the present disclosure does not comprise the coding sequence of (e.g., a transgene encoding) a Serine Protease Inhibitor Kazal-type (SPINK) polypeptide (e.g., a human SPINK polypeptide, such as a SPINK5 polypeptide). In some embodiments, a polynucleotide of the present disclosure does not comprise the coding sequence of (e.g., a transgene encoding) a filaggrin or filaggrin 2 polypeptide (e.g., a human filaggrin or filaggrin 2 polypeptide). In some embodiments, a polynucleotide of the present disclosure does not comprise the coding sequence of (e.g., a transgene encoding) a Cystic Fibrosis Transmembrane Conductance Regulator (CFTR) polypeptide (e.g., a human CFTR polypeptide). In some embodiments, a polynucleotide of the present disclosure does not comprise the coding sequence of (e.g., a transgene encoding) an ichthyosis-associated polypeptide (e.g., an ATP-binding cassette sub-family A member 12 polypeptide, a 1-acylglycerol-3-phosphate O-acyltransferase ABHD5 polypeptide, an Aldehyde dehydrogenase family 3 member A2 polypeptide, an Arachidonate 12-lipoxygenase 12R-type polypeptide, a Hydroperoxide isomerase ALOXE3 polypeptide, an AP-1 complex subunit sigma-1A polypeptide, an Arylsulfatase E polypeptide, a Caspase-14 polypeptide, a Corneodesmosin polypeptide, a Ceramide synthase 3 polypeptide, a Carbohydrate sulfotransferase 8 polypeptide, a Claudin-1 polypeptide, a Cystatin-A polypeptide, a Cytochrome P450 4F22 polypeptide, a 3-beta-hydroxysteroid-Delta (8),Delta (7)-isomerase polypeptide, an Elongation of very long chain fatty acids protein 4 polypeptide, a Filaggrin polypeptide, a Filaggrin 2 polypeptide, a Gap junction beta-2 polypeptide, a Gap junction beta-3 polypeptide, a Gap junction beta-4 polypeptide, a Gap junction beta-6 polypeptide, a 3-ketodihydrosphingosine reductase polypeptide, a Keratin, type II cytoskeletal 1 polypeptide, a Keratin, type II cytoskeletal 2 epidermal polypeptide, a Keratin, type I cytoskeletal 9 polypeptide, a Keratin, type I cytoskeletal 10 polypeptide, a Lipase member N polypeptide, a Loricrin polypeptide, a Membrane-bound transcription factor site-2 protease polypeptide, a Magnesium transporter NIPA4 polypeptide, a Sterol-4-alpha-carboxylate 3-dehydrogenase, decarboxylating polypeptide, a Peroxisomal targeting signal 2 receptor polypeptide, a D-3-phosphoglycerate dehydrogenase polypeptide, a Phytanoyl-CoA dioxygenase, peroxisomal polypeptide, Patatin-like phospholipase domain-containing protein 1 polypeptide, a Proteasome maturation protein polypeptide, a Phosphoserine aminotransferase polypeptide, a Short-chain dehydrogenase/reductase family 9C member 7 polypeptide, a Serpin B8 polypeptide, a Long-chain fatty acid transport protein 4 polypeptide, a Synaptosomal-associated protein 29 polypeptide, a Suppressor of tumorigenicity 14 protein polypeptide, a Steryl-sulfatase polypeptide, a Vacuolar protein sorting-associated protein 33B polypeptide, and a CAAX prenyl protease 1 homolog polypeptide). In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, and/or any chimeric polypeptides thereof. In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, a transglutaminase (TGM) polypeptide, a filaggrin polypeptide, a cosmetic protein, an antibody, a SPINK polypeptide, a CFTR polypeptide, an ichthyosis-associated polypeptide, an Alpha-1-antitrypsin polypeptide, a Sodium-dependent phosphate transport protein 2B polypeptide, a Dynein heavy chain 5 axonemal polypeptide, a Dynein heavy chain 11 axonemal polypeptide, a Coiled-coil domain-containing protein 39 polypeptide, a Dynein intermediate chain 1 axonemal polypeptide, a Coiled-coil domain-containing protein 40 polypeptide, a Coiled-coil domain containing protein 103 polypeptide, a Sperm-associated antigen 1 polypeptide, a Zinc finger MYND domain-containing protein 10 polypeptide, an Armadillo repeat containing protein 4 polypeptide, a Coiled-coil domain-containing protein 151 polypeptide, a Dynein intermediate chain 2 axonemal polypeptide, a Radial spoke head 1 homolog polypeptide, a Coiled-coil domain-containing protein 114 polypeptide, a Radial spoke head protein 4 homolog A polypeptide, a Dynein assembly factor 1 axonemal polypeptide, a Dynein assembly factor 2 axonemal polypeptide, a Leucine-rich repeat-containing protein 6 polypeptide, a Pulmonary surfactant-associated protein B polypeptide, a Pulmonary surfactant-associated protein C polypeptide, a Homeobox protein Nkx-2.1 polypeptide, an ATP-binding cassette sub-family A member 3 polypeptide, a Cytokine receptor common subunit beta polypeptide, a Granulocyte-macrophage colony-stimulating factor receptor subunit alpha polypeptide, a Bone morphogenetic protein receptor type-2 polypeptide, a Sarcoplasmic/endoplasmic reticulum calcium ATPase 2 polypeptide, a serine/threonine-protein kinase receptor R3 polypeptide, an Endoglin polypeptide, a Mothers against decapentaplegic homolog 9 polypeptide, a Caveolin-1 polypeptide, a Potassium channel subfamily K member 3 polypeptide, an elF-2-alpha kinase GCN2 polypeptide, a Pulmonary surfactant-associated protein A2 polypeptide, a Telomerase reverse transcriptase polypeptide, a Dyskerin polypeptide, a Regulator of telomere elongation helicase 1 polypeptide, a Poly (A)-specific ribonuclease PARN polypeptide, a TERF1-interacting nuclear factor 2 polypeptide, an H/ACA ribonucleoprotein complex non-core subunit NAF1 polypeptide, a Mucin-5B polypeptide, a Desmoplakin polypeptide, a CST complex subunit STN1 polypeptide, a Dipeptidyl peptidase 9 polypeptide, and/or any chimeric polypeptides thereof.

In some embodiments, a polynucleotide of the present disclosure does not comprise the coding sequence of (e.g., a transgene encoding) an immunomodulatory polypeptide. In some embodiments, a polynucleotide of the present disclosure does not comprise the coding sequence of (e.g., a transgene encoding) a cytokine polypeptide and/or a chemokine polypeptide. In some embodiments, a polynucleotide of the present disclosure does not comprise the coding sequence of (e.g., a transgene encoding) a cytokine polypeptide. In some embodiments, a polynucleotide of the present disclosure does not comprise the coding sequence of (e.g., a transgene encoding) an IL-1 polypeptide, an IL-2 polypeptide, an IL-7 polypeptide, an IL-12 polypeptide, an IL-13 polypeptide, an IL-15 polypeptide, an IL-17 polypeptide, an IL-18 polypeptide, an IL-28 polypeptide, an IL-32 polypeptide, an IL-33 polypeptide, an IL-34 polypeptide, a TNFα polypeptide, an IFNγ polypeptide, a G-CSF polypeptide, a GM-CSF polypeptide, and/or any chimeric polypeptides thereof.

In some embodiments, a polynucleotide of the present disclosure does not comprise the coding sequence of (e.g., a transgene encoding) a chemokine polypeptide. In some embodiments, a polynucleotide of the present disclosure does not comprise the coding sequence of (e.g., a transgene encoding) a CXCL1 polypeptide, a CXCL2 polypeptide, a CXCL8 polypeptide, a CXCL9 polypeptide, a CXCL11 polypeptide, a CXCL16 polypeptide, a CCL2 polypeptide, a CCL3 polypeptide, a CCL4 polypeptide, a CCL5 polypeptide, a CCL11 polypeptide, and/or any chimeric polypeptides thereof.

In some embodiments, a polynucleotide of the present disclosure does not comprise the coding sequence of (e.g., a transgene encoding) a sterile alpha motif domain-containing protein 11 polypeptide, a nephrocystin-4 polypeptide, an espin polypeptide, a nicotinamide/nicotinic acid mononucleotide adenylyltransferase 1 polypeptide, a mitofusin-2 polypeptide, an ER membrane protein complex subunit 1 polypeptide, a phospholipase A2 group V polypeptide, a dehydrodolichyl diphosphate synthase complex subunit polypeptide, a palmitoyl-protein thioesterase 1 polypeptide, an elongation of very long chain fatty acids protein 1 polypeptide, a protein O-linked-mannose beta-1,2-N-acetylglucosaminyltransferase 1 polypeptide, a retinoid isomerohydrolase polypeptide, a retinal-specific phospholipid-transporting ATPase ABCA4 polypeptide, a Collagen alpha-1 (XI) polypeptide, a Guanine nucleotide-binding protein G (t) subunit alpha-2 polypeptide, a Chloride channel CLIC-like protein 1 polypeptide, a DNA damage-regulated autophagy modulator protein 2 polypeptide, an U4/U6 small nuclear ribonucleoprotein Prp3 polypeptide, an Alpha-endosulfine polypeptide, a Semaphorin-4A polypeptide, a Cyclic AMP-dependent transcription factor ATF-6 alpha polypeptide, a Hemicentin-1 polypeptide, a Complement factor H polypeptide, a Protein crumbs homolog 1 polypeptide, an Adiponectin receptor protein 1 polypeptide, a Protein RD3 polypeptide, a Serine/threonine-protein kinase Nek2 polypeptide, a Feline leukemia virus subgroup C receptor-related protein 1 polypeptide, an Usherin polypeptide, a Serologically defined colon cancer antigen 8 polypeptide, an Olfactory receptor 2W3 polypeptide, a NBAS subunit of NRZ tethering complex polypeptide, a Cytosolic carboxypeptidase-like protein 5 polypeptide, a Zinc finger protein 513 polypeptide, an Intraflagellar transport protein 172 homolog polypeptide, a Photoreceptor cilium actin regulator polypeptide, an EGF-containing fibulin-like extracellular matrix protein 1/TLE family member 5 polypeptide, a Protein FAM161A polypeptide, a WD repeat-containing and planar cell polarity effector protein fritz homolog polypeptide, a Centrosome-associated protein ALMS1 polypeptide, an U5 small nuclear ribonucleoprotein 200 kDa helicase polypeptide, a Metal transporter CNNM4 polypeptide, a Cyclic nucleotide-gated cation channel alpha-3 polypeptide, a Nephrocystin-1 polypeptide, a Tyrosine-protein kinase Mer polypeptide, a Bardet-Biedl syndrome 5 protein polypeptide, a Ceramide kinase-like protein polypeptide, a Neurogenic differentiation factor 1 polypeptide, a Transmembrane protein 237 polypeptide, an Inward rectifier potassium channel 13 polypeptide, a S-arrestin polypeptide, a Secreted phosphoprotein 24 polypeptide, a CCA tRNA nucleotidyltransferase 1 polypeptide, a mitochondrial, Sodium bicarbonate cotransporter 3 polypeptide, a Leucine zipper transcription factor-like protein 1 polypeptide, a Guanine nucleotide-binding protein G (t) subunit alpha-1 polypeptide, a Three-prime repair exonuclease 1 polypeptide, a MAP kinase-activated protein kinase 3 polypeptide, an Ataxin-7 polypeptide, a Vitamin K-dependent protein S polypeptide, an ADP-ribosylation factor-like protein 6 polypeptide, an Interphotoreceptor matrix proteoglycan 2 polypeptide, an IQ calmodulin-binding motif-containing protein 1 polypeptide, a Rhodopsin polypeptide, a Nephrocystin-3 polypeptide, a Clarin-1 polypeptide, a Probable cationic amino acid transporter/Solute carrier family 7 member 14 polypeptide, a Choline-phosphate cytidylyltransferase A polypeptide, a Centrosomal protein of 19 kDa polypeptide, a Rod cGMP-specific 3′,5′-cyclic phosphodiesterase subunit beta polypeptide, a Wolframin, Homeobox protein HMX1 polypeptide, a Ras-related protein Rab-28 polypeptide, a Coiled-coil and C2 domain-containing protein 2A polypeptide, a Prominin-1 polypeptide, an Adhesion G protein-coupled receptor A3 polypeptide, a Death domain-containing protein 1 polypeptide, a WD repeat-containing protein 19 polypeptide, a cGMP-gated cation channel alpha-1 polypeptide, a CDGSH iron-sulfur domain-containing protein 2 polypeptide, a Microsomal triglyceride transfer protein large subunit polypeptide, a Leucine-rich repeat, immunoglobulin-like domain and transmembrane domain-containing protein 3 polypeptide, a Bardet-Biedl syndrome 7 protein polypeptide, a Bardet-Biedl syndrome 12 protein polypeptide, a Major facilitator superfamily domain-containing protein 8 polypeptide, a Serine/threonine-protein kinase PLK4 polypeptide, a Lecithin retinol acyltransferase polypeptide, a Toll-like receptor 3 polypeptide, a Cytochrome P450 4V2 polypeptide, a Spliceosome-associated protein CWC27 homolog polypeptide, a Centrosomal protein POC5 polypeptide, a Versican core protein polypeptide, an Adhesion G-protein coupled receptor V1 polypeptide, a COUP transcription factor 1 polypeptide, a Mitochondrial outer membrane protein SLC25A46 polypeptide, a Catenin alpha-1 polypeptide, a Histidine—tRNA ligase, cytoplasmic polypeptide, a Rod cGMP-specific 3′,5′-cyclic phosphodiesterase subunit alpha polypeptide, a Metabotropic glutamate receptor 6 polypeptide, a Serine/threonine-protein kinase MAK polypeptide, a Complement C2 polypeptide, a Complement factor B polypeptide, a Tubby-related protein 1 polypeptide, a Guanylyl cyclase-activating protein 1 polypeptide, a Guanylyl cyclase-activating protein 2 polypeptide, a Peripherin-2 polypeptide, an Interphotoreceptor matrix proteoglycan 1 polypeptide, a Protein eyes shut homolog polypeptide, a Collagen alpha-1 (IX) chain polypeptide, a Regulating synaptic membrane exocytosis protein 1 polypeptide, a Lebercilin polypeptide, an Elongation of very long chain fatty acids protein 4 polypeptide, a PR domain zinc finger protein 13 polypeptide, a Reticulon-4-interacting protein 1, mitochondrial, polypeptide, a Jouberin polypeptide, a Peroxisomal targeting signal 2 receptor polypeptide, a CCR4-NOT transcription complex subunit 9 polypeptide, an Aryl hydrocarbon receptor polypeptide, a Kelch-like protein 7 polypeptide, a Retinitis pigmentosa 9 protein polypeptide, a Protein PTHB1 polypeptide, a Peroxisomal ATPase PEX1 polypeptide, a Tetraspanin-12 polypeptide, an Inosine-5′-monophosphate dehydrogenase 1 polypeptide, a Short-wave-sensitive opsin 1 polypeptide, an UPF0606 protein KIAA1549 polypeptide, a Retinitis pigmentosa 1-like 1 protein polypeptide, a Disintegrin and metalloproteinase domain-containing protein 9 polypeptide, a Heparan-alpha-glucosaminide N-acetyltransferase polypeptide, an Oxygen-regulated protein 1 polypeptide, an Alpha-tocopherol transfer protein polypeptide, a Centrosome and spindle pole-associated protein 1 polypeptide, a Dynamin-like 120 kDa protein, mitochondrial, polypeptide, a Peroxisome biogenesis factor 2 polypeptide, a Cyclic nucleotide-gated cation channel beta-3 polypeptide, a Cilia- and flagella-associated protein 418 polypeptide, a Growth/differentiation factor 6 polypeptide, a Regulating synaptic membrane exocytosis protein 2 polypeptide, a Potassium voltage-gated channel subfamily V member 2 polypeptide, an E3 ubiquitin-protein ligase Topors polypeptide, a Centrosomal protein of 78 kDa polypeptide, an Inversin polypeptide, an U4/U6 small nuclear ribonucleoprotein Prp4 polypeptide, a Whirlin polypeptide, an E3 ubiquitin-protein ligase TRIM32 polypeptide, a Toll-like receptor 4 polypeptide, a Cytoplasmic dynein 2 intermediate chain 2 polypeptide, a Programmed cell death protein 2 polypeptide, an Exosome complex component RRP4 polypeptide, a Phosphatidylinositol polyphosphate 5-phosphatase type IV polypeptide, a Phytanoyl-CoA dioxygenase, peroxisomal, polypeptide, an Acyl-CoA-binding domain-containing protein polypeptide, a Protocadherin-15 polypeptide, a Retinol-binding protein 3 polypeptide, a DNA excision repair protein ERCC-6 polypeptide, a Hexokinase-1 polypeptide, a Cadherin-23 polypeptide, a Cadherin-related family member 1 polypeptide, a RPE-retinal G protein-coupled receptor polypeptide, a Kinesin-like protein KIF11 polypeptide, a Retinol-binding protein 4 polypeptide, a Cone cGMP-specific 3′,5′-cyclic phosphodiesterase subunit alpha polypeptide, a Paired box protein Pax-2 polypeptide, a PDZ domain-containing protein 7 polypeptide, an ADP-ribosylation factor-like protein 3 polypeptide, a BBSome-interacting protein 1 polypeptide, an Age-related maculopathy susceptibility protein 2 polypeptide, a Serine protease HTRA1 polypeptide, an Ornithine aminotransferase, mitochondrial, polypeptide, a Zinc finger protein 408 polypeptide, a Tubby protein homolog polypeptide, a Transcriptional enhancer factor TEF-1 polypeptide, a Harmonin polypeptide, a Transmembrane protein 216 polypeptide, a Bestrophin-1 polypeptide, an Isoaspartyl peptidase/L-asparaginase polypeptide, a Rod outer segment membrane protein 1 polypeptide, a Bardet-Biedl syndrome 1 protein polypeptide, a Calcium-binding protein 4 polypeptide, a Low-density lipoprotein receptor-related protein 5 polypeptide, a Calpain-5 polypeptide, an Unconventional myosin-VIIa polypeptide, a Transmembrane protein 126A polypeptide, a Frizzled-4 polypeptide, a Cytoplasmic dynein 2 light intermediate chain 1 polypeptide, a Centrosomal protein of 164 kDa polypeptide, a Complement Clq tumor necrosis factor-related protein 5 polypeptide, a Membrane frizzled-related protein polypeptide, a Voltage-dependent calcium channel subunit alpha-2/delta-4 polypeptide, a Guanine nucleotide-binding protein G (I)/G(S)/G (T) subunit beta-3 polypeptide, a Retinal cone rhodopsin-sensitive cGMP 3′,5′-cyclic phosphodiesterase subunit gamma polypeptide, a Matrix metalloproteinase-19 polypeptide, a Retinol dehydrogenase 5 polypeptide, a T-complex protein 1 subunit beta polypeptide, a Bardet-Biedl syndrome 10 protein polypeptide, a Centrosomal protein of 290 kDa polypeptide, a POC1 centriolar protein homolog B polypeptide, a Mevalonate kinase polypeptide, an Intraflagellar transport protein 81 homolog polypeptide, a Mitochondrial translation release factor in rescue polypeptide, an Integral membrane protein 2B polypeptide, a Retinoblastoma-associated protein polypeptide, a RCC1 and BTB domain-containing protein 1 polypeptide, a Rhodopsin kinase GRK1 polypeptide, an X-linked retinitis pigmentosa GTPase regulator-interacting protein 1 polypeptide, a Neural retina-specific leucine zipper protein polypeptide, a Homeobox protein OTX2 polypeptide, a Retinol dehydrogenase 11 polypeptide, a Retinol dehydrogenase 12 polypeptide, a Tubulin polyglutamylase TTLL5 polypeptide, a Spermatogenesis-associated protein 7 polypeptide, a Tetratricopeptide repeat protein 8 polypeptide, a Fibulin-5 polypeptide, a Transient receptor potential cation channel subfamily M member 1 polypeptide, a Gamma-tubulin complex component 4 polypeptide, a Sodium/potassium/calcium exchanger 1 polypeptide, a Photoreceptor-specific nuclear receptor polypeptide, a Bardet-Biedl syndrome 4 protein polypeptide, a Calcium and integrin-binding family member 2 polypeptide, a Retinaldehyde-binding protein 1 polypeptide, a N-acetylglucosamine-1-phosphotransferase subunit gamma polypeptide, an Intraflagellar transport protein 140 homolog polypeptide, a Clusterin-associated protein 1 polypeptide, an ATP-binding cassette sub-family C member 6 polypeptide, a Ketimine reductase mu-crystallin polypeptide, a Battenin polypeptide, a Zinc finger protein 423 polypeptide, a Protein fantom polypeptide, a Bardet-Biedl syndrome 2 protein polypeptide, an ADP-ribosylation factor-like protein 2-binding protein polypeptide, a Cyclic nucleotide-gated cation channel beta-1 polypeptide, a Cadherin-3 polypeptide, a Pre-mRNA-splicing factor ATP-dependent RNA helicase PRP16 polypeptide, a disintegrin and metalloproteinase with thrombospondin motifs 18 polypeptide, a Solute carrier family 38 member 8 polypeptide, a Retinal guanylyl cyclase 1 polypeptide, a Pre-mRNA-processing-splicing factor 8 polypeptide, an Aryl-hydrocarbon-interacting protein-like 1 polypeptide, a Membrane-associated phosphatidylinositol transfer protein 3 polypeptide, a Protein unc-119 homolog A polypeptide, a Probable G-protein coupled receptor 179 polypeptide, a Tectonic-like complex member MKS1 polypeptide, a Carbonic anhydrase 4 polypeptide, a Regulator of G-protein signaling 9 polypeptide, an Arylsulfatase G polypeptide, a pre-mRNA splicing regulator USH1G polypeptide, a Photoreceptor disk component PRCD polypeptide, a Fascin-2 polypeptide, a Retinal rod rhodopsin-sensitive cGMP 3′,5′-cyclic phosphodiesterase subunit gamma polypeptide, a Laminin subunit alpha-1 polypeptide, an AFG3-like protein 2 polypeptide, a Cone-rod homeobox protein polypeptide, a Receptor expression-enhancing protein 6 polypeptide, a Retina and anterior neural fold homeobox protein 2 polypeptide, a Complement C3 polypeptide, a Rho guanine nucleotide exchange factor 18 polypeptide, a Patatin-like phospholipase domain-containing protein 6 polypeptide, a Regulator of G-protein signaling 9-binding protein polypeptide, an Optic atrophy 3 protein polypeptide, an U4/U6 small nuclear ribonucleoprotein Prp31 polypeptide, an Isocitrate dehydrogenase [NAD] subunit beta, mitochondrial, polypeptide, a Pantothenate kinase 2, mitochondrial, polypeptide, a Protein jagged-1 polypeptide, a Molecular chaperone MKKS polypeptide, a Centrosomal protein kizuna, Lysophosphatidylserine lipase ABHD12 polypeptide, a Kinesin-like protein KIF3B polypeptide, a Centrosome-associated protein CEP250 polypeptide, a Pre-mRNA-processing factor 6 polypeptide, a Cilia- and flagella-associated protein 410 polypeptide, a Dynamin-1-like protein polypeptide, a Metalloproteinase inhibitor 3 polypeptide, an Intraflagellar transport protein 27 homolog polypeptide, a Fibulin-1 polypeptide, a MIEF1 upstream open reading frame protein polypeptide, an Aconitate hydratase, mitochondrial, polypeptide, a Gamma-tubulin complex component 6 polypeptide, a Centriole and centriolar satellite protein polypeptide, a Retinoschisin, Protein XRP2 polypeptide, a Dystrophin polypeptide, an X-linked retinitis pigmentosa GTPase regulator polypeptide, a Nyctalopin polypeptide, an Xaa-Pro dipeptidase polypeptide, a Norrin polypeptide, a Voltage-dependent L-type calcium channel subunit alpha-1F polypeptide, a Phosphoglycerate kinase 1 polypeptide, a Rab proteins geranylgeranyltransferase component A 1 polypeptide, a Mitochondrial import inner membrane translocase subunit Tim8 A polypeptide, a Ribose-phosphate pyrophosphokinase 1 polypeptide, a Long-wave-sensitive opsin 1 polypeptide, a Medium-wave-sensitive opsin 1 polypeptide, a Short-wave-sensitive opsin 1 polypeptide, a Transcription factor A, mitochondrial, polypeptide, a NADH-ubiquinone oxidoreductase chain 1 polypeptide, a NADH-ubiquinone oxidoreductase chain 2 polypeptide, a NADH-ubiquinone oxidoreductase chain 3 polypeptide, a NADH-ubiquinone oxidoreductase chain 4L polypeptide, a NADH-ubiquinone oxidoreductase chain 4 polypeptide, a NADH-ubiquinone oxidoreductase chain 5 polypeptide, a NADH-ubiquinone oxidoreductase chain 6 polypeptide, an ATP synthase subunit a polypeptide, an ATP synthase protein 8 polypeptide, a Cytochrome c oxidase subunit 1 polypeptide, a Cytochrome c oxidase subunit 3 polypeptide, a Cytochrome b polypeptide, a Leucine—tRNA ligase, mitochondrial, polypeptide, a Nondiscriminating glutamyl-tRNA synthetase EARS2, mitochondrial, polypeptide, a Lysine—tRNA ligase polypeptide, a Histidine-RNA ligase, mitochondrial, polypeptide, aSerine-tRNA ligase, mitochondrial, polypeptide, a Probable proline-tRNA ligase, mitochondrial, polypeptide, a Cyanocobalamin reductase/alkylcobalamin dealkylase polypeptide, a POU domain, class 3, transcription factor 4 polypeptide, a Ribosomal protein S6 kinase alpha-6 polypeptide, a Ciliogenesis and planar polarity effector 1 polypeptide, a Meckelin, TRAF3-interacting protein 1 polypeptide, an Intraflagellar transport protein 74 homolog polypeptide, an S phase cyclin A-associated protein in the endoplasmic reticulum polypeptide, a Sodium channel and clathrin linker 1 polypeptide, a Protein TALPID3 polypeptide, a Tectonic-2 polypeptide, an ADP-ribosylation factor-like protein 13B polypeptide, a B9 domain-containing protein 1 polypeptide, a B9 domain-containing protein 2 polypeptide, a C2 domain-containing protein 3 polypeptide, a Centrosomal protein of 41 kDa polypeptide, a Centrosomal protein of 104 kDa polypeptide, a Centrosomal protein of 120 kDa polypeptide, an Intraflagellar transport protein 172 homolog polypeptide, a Katanin-interacting protein polypeptide, a Kinesin-like protein KIF7 polypeptide, a Retinal rod rhodopsin-sensitive cGMP 3′,5′-cyclic phosphodiesterase subunit delta polypeptide, a Tectonic-1 polypeptide, a Tectonic-3 polypeptide, a Transmembrane protein 107 polypeptide, a Transmembrane protein 138 polypeptide, a Transmembrane protein 231 polypeptide, a Tetratricopeptide repeat protein 21B polypeptide, a Nuclear receptor ROR-alpha polypeptide, a Beta-nerve growth factor polypeptide, a Solute carrier family 4 member 11 polypeptide, a Zinc finger E-box-binding homeobox 1 polypeptide, a Keratin, type II cuticular Hb3 polypeptide, a Keratin, type I cytoskeletal 12 polypeptide, a Transforming growth factor-beta-induced protein ig-h3 polypeptide, a Tumor-associated calcium signal transducer 2 polypeptide, a Carbohydrate sulfotransferase 6 polypeptide, a Gelsolin polypeptide, an UbiA prenyltransferase domain-containing protein 1 polypeptide, a Decorin polypeptide, a 1-phosphatidylinositol 3-phosphate 5-kinase polypeptide, a Transcription factor Ovo-like 2 polypeptide, a Grainyhead-like protein 2 homolog polypeptide, and/or any chimeric polypeptides thereof.

### Chimeric Polypeptides

In some embodiments, a polynucleotide of the present disclosure encodes a chimeric polypeptide comprising a first ATPase polypeptide (e.g., a Calcium-transporting ATPase type 2C member 1 polypeptide or a Sarcoplasmic/endoplasmic reticulum calcium ATPase 2 polypeptide) and a second ATPase polypeptide (e.g., a Calcium-transporting ATPase type 2C member 1 polypeptide or a Sarcoplasmic/endoplasmic reticulum calcium ATPase 2 polypeptide). In some embodiments, the first and second ATPase polypeptides are the same. In some embodiments, the first and second ATPase polypeptides are different. In some embodiments, the chimeric polypeptide further comprises a linker polypeptide linking the first and second polypeptides. In some embodiments, the chimeric polypeptide comprises, from n-terminus to c-terminus, the first ATPase polypeptide—the linker polypeptide—the second ATPase polypeptide. The first and/or second polypeptides may be any of the ATPase polypeptides described herein or known in the art.

In some embodiments, the linker polypeptide is a cleavable linker polypeptide. Any cleavable linker polypeptide known in the art may be used in the chimeric polypeptides of the present disclosure, including, for example, a T2A linker, a P2A linker, a E2A linker, and F2A linker, etc. In some embodiments, the linker polypeptide is a T2A linker polypeptide. An exemplary nucleic acid sequence encoding a T2A linker polypeptide is provided as SEQ ID NO: 7. An exemplary amino acid sequence of a T2A linker polypeptide is provided as SEQ ID NO: 11. In some embodiments, the linker polypeptide is a P2A linker polypeptide. An exemplary nucleic acid sequence encoding a P2A linker polypeptide is provided as SEQ ID NO: 8. An exemplary amino acid sequence of a P2A linker polypeptide is provided as SEQ ID NO: 12. In some embodiments, the linker polypeptide is an E2A linker polypeptide. An exemplary nucleic acid sequence encoding an E2A linker polypeptide is provided as SEQ ID NO: 9. An exemplary amino acid sequence of an E2A linker polypeptide is provided as SEQ ID NO: 13. In some embodiments, the linker polypeptide is an F2A linker polypeptide. An exemplary nucleic acid sequence encoding an F2A linker polypeptide is provided as SEQ ID NO: 10. An exemplary amino acid sequence of an F2A linker polypeptide is provided as SEQ ID NO: 14. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 11-14. In some embodiments, the linker polypeptide comprises a sequence selected from SEQ ID NOS: 11-14.

In some embodiments, the linker polypeptide is a non-cleavable linker polypeptide. Any non-cleavable linker polypeptide known in the art may be used in the chimeric polypeptides of the present disclosure, including, for example, a GGGGSGGGGSGGGGS (SEQ ID NO: 15) linker, a GGSSRSSSSGGGGSGGGG (SEQ ID NO: 16) linker, a GGGGSGGGGSGGGGSGGGGS (SEQ ID NO: 17) linker, a CGGGSGGGGSGGGGS (SEQ ID NO: 18) linker, a SHGGHGGGGSGGGGS (SEQ ID NO: 19) linker, a MGGMSGGGGSGGGGS (SEQ ID NO: 20) linker, a YGGYSGGGGSGGGGS (SEQ ID NO: 21) linker, a WGGYSGGGGSGGGGS (SEQ ID NO: 22) linker, a SVSVGMKPSPRP (SEQ ID NO: 23) linker, a VISNHAGSSRRL (SEQ ID NO: 24) linker, a PWIPTPRPTFTG (SEQ ID NO: 25) linker, a RGRGRGRGRGR (SEQ ID NO: 26) linker, etc. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-26. In some embodiments, the linker polypeptide comprises a sequence selected from SEQ ID NOS: 15-26.

### Recombinant Nucleic Acids

In some embodiments, the present disclosure relates to recombinant nucleic acids comprising any one or more of the polynucleotides described herein. In some embodiments, the recombinant nucleic acid is a vector (e.g., an expression vector, a display vector, etc.). In some embodiments, the vector is a DNA vector or an RNA vector. Generally, vectors suitable to maintain, propagate, and/or express polynucleotides to produce one or more polypeptides in a subject may be used. Examples of suitable vectors may include, for example, plasmids, cosmids, episomes, transposons, and viral vectors (e.g., adenoviral vectors, adeno-associated viral vectors, vaccinia viral vectors, Sindbis-viral vectors, measles vectors, herpes viral vectors, lentiviral vectors, retroviral vectors, etc.). In some embodiments, the vector is a herpes viral vector. In some embodiments, the vector is capable of autonomous replication in a host cell. In some embodiments, the vector is incapable of autonomous replication in a host cell. In some embodiments, the vector can integrate into a host DNA. In some embodiments, the vector cannot integrate into a host DNA (e.g., is episomal). Methods of making vectors containing one or more polynucleotides of interest are well known to one of ordinary skill in the art, including, for example, by chemical synthesis or by artificial manipulation of isolated segments of nucleic acids (e.g., by genetic engineering techniques).

In some embodiments, a recombinant nucleic acid of the present disclosure is a herpes simplex virus (HSV) amplicon. Herpes virus amplicons, including the structural features and methods of making the same, are generally known to one of ordinary skill in the art (see e.g., de Silva S. and Bowers W. “Herpes Virus Amplicon Vectors”. Viruses 2009, 1, 594-629). In some embodiments, the herpes simplex virus amplicon is an HSV-1 amplicon. In some embodiments, the herpes simplex virus amplicon is an HSV-1 hybrid amplicon. Examples of HSV-1 hybrid amplicons may include, but are not limited to, HSV/AAV hybrid amplicons, HSV/EBV hybrid amplicons, HSV/EBV/RV hybrid amplicons, and/or HSV/Sleeping Beauty hybrid amplicons. In some embodiments, the amplicon is an HSV/AAV hybrid amplicon. In some embodiments, the amplicon is an HSV/Sleeping Beauty hybrid amplicon.

In some embodiments, a recombinant nucleic acid of the present disclosure is a recombinant herpes virus genome. The recombinant herpes virus genome may be a recombinant genome from any member of the Herpesviridae family of DNA viruses known in the art, including, for example, a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Epstein-Barr virus genome, a recombinant Kaposi's sarcoma-associated herpesvirus genome, and any combinations or derivatives thereof. In some embodiments, the recombinant herpes virus genome comprises one or more (e.g., one or more, two or more, three or more, four or more, five or more, six or more, seven or more, eight or more, nine or more, ten or more, etc.) inactivating mutations. As used herein, an “inactivating mutation” may refer to any mutation that results in a gene or regulon product (RNA or protein) having reduced, undetectable, or eliminated quantity and/or function (e.g., as compared to a corresponding sequence lacking the inactivating mutation). Examples of inactivating mutations may include, but are not limited to, deletions, insertions, point mutations, and rearrangements in transcriptional control sequences (promoters, enhancers, insulators, etc.) and/or coding sequences of a given gene or regulon. Any suitable method of measuring the quantity of a gene or regulon product known in the art may be used, including, for example, qPCR, Northern blots, RNAseq, western blots, ELISAs, etc. In some embodiments, the one or more inactivating mutations are in one or more (e.g., one or more, two or more, three or more, four or more, five or more, six or more, seven or more, eight or more, nine or more, ten or more, etc.) herpes virus genes. In some embodiments, the recombinant herpes virus genome is attenuated (e.g., as compared to a corresponding, wild-type herpes virus genome). In some embodiments, the recombinant herpes virus genome is replication competent. In some embodiments, the recombinant herpes virus genome is replication defective. In some embodiments, the recombinant herpes virus genome is not oncolytic.

In some embodiments, the recombinant nucleic acid is a recombinant herpes simplex virus (HSV) genome. In some embodiments, the recombinant herpes simplex virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes simplex virus genes. In some embodiments, the recombinant herpes simplex virus genome is attenuated (e.g., as compared to a corresponding, wild-type herpes simplex virus genome). In some embodiments, the recombinant herpes simplex virus genome is replication competent. In some embodiments, the recombinant herpes simplex virus genome is replication defective. In some embodiments, the recombinant herpes simplex virus genome is not oncolytic.

In some embodiments, the recombinant herpes virus genome is a recombinant herpes simplex virus type 1 (HSV-1) genome, a recombinant herpes simplex virus type 2 (HSV-2) genome, or any derivatives thereof. In some embodiments, the recombinant herpes simplex virus genome is a recombinant HSV-1 genome. In some embodiments, the recombinant HSV-1 genome may be from any HSV-1 strain known in the art, including, for example, strains 17, Ty25, R62, S25, Ku86, S23, R11, Ty148, Ku47, H166syn, 1319-2005, F-13, M-12, 90237, F-17, KOS, 3083-2008, F12g, L2, CD38, H193, M-15, India 2011, 0116209, F-11I, 66-207, 2762, 369-2007, 3355, MacIntyre, McKrae, 7862, 7-hse, HF10, 1394,2005, 270-2007, OD4, SC16, M-19, 4J1037, 5J1060, J1060, KOS79, 132-1988, 160-1982, H166, 2158-2007, RE, 78326, F18g, F11, 172-2010, H129, F, E4, CJ994, F14g, E03, E22, E10, E06, E11, E25, E23, E35, E15, E07, E12, E14, E08, E19, E13, ATCC 2011, etc. (see e.g., Bowen et al. J Virol. 2019 Apr. 3;93 (8)). In some embodiments, the recombinant HSV-1 genome is from the KOS strain. In some embodiments, the recombinant HSV-1 genome is not from the McKrae strain. In some embodiments, the recombinant HSV-1 genome is attenuated (e.g., as compared to a corresponding, wild-type HSV-1 genome). In some embodiments, the recombinant HSV-1 genome is replication competent. In some embodiments, the recombinant HSV-1 genome is replication defective. In some embodiments, the recombinant HSV-1 genome is not oncolytic.

In some embodiments, the recombinant herpes simplex virus genome comprises an inactivating mutation in at least one, at least two, at least three, at least four, at least five, at least six, at least seven, or all eight of the Infected Cell Protein (or Infected Cell Polypeptide) (ICP) 0, ICP4, ICP22, ICP27, ICP47, thymidine kinase (tk), Long Unique Region (UL) 41 and/or UL55 herpes simplex virus genes. In some embodiments, the recombinant herpes simplex virus genome does not comprise an inactivating mutation in the ICP34.5 (one or both copies) and/or ICP47 herpes simplex virus genes (e.g., to avoid production of an immune-stimulating virus). In some embodiments, the recombinant herpes simplex virus genome does not comprise an inactivating mutation in the ICP34.5 (one or both copies) herpes simplex virus gene. In some embodiments, the recombinant herpes simplex virus genome does not comprise an inactivating mutation in the ICP47 herpes simplex virus gene. In some embodiments, the recombinant herpes simplex virus genome does not comprise an inactivating mutation in the ICP34.5 (one or both copies) and ICP47 herpes simplex virus genes. In some embodiments, the recombinant herpes simplex virus genome is not oncolytic. In some embodiments, the recombinant herpes simplex virus genome is not conditionally replication competent. In some embodiments, the recombinant herpes simplex virus genome is not conditionally replication competent in a cancerous cell.

In some embodiments, the recombinant herpes simplex virus genome comprises an inactivating mutation in the ICPO gene (one or both copies). In some embodiments, the recombinant herpes simplex virus genome comprises an inactivating mutation in the ICPO gene (one or both copies) and further comprises an inactivating mutation in the ICP4 (one or both copies), ICP22, ICP27, ICP47, UL41, and/or UL55 genes. In some embodiments, the recombinant herpes simplex virus genome comprises an inactivating mutation in the ICPO gene (one or both copies), and an inactivating mutation in the ICP4 gene (one or both copies). In some embodiments, the recombinant herpes simplex virus genome comprises an inactivating mutation in the ICPO gene (one or both copies), and an inactivating mutation in the ICP22 gene. In some embodiments, the recombinant herpes simplex virus genome comprises an inactivating mutation in the ICPO gene (one or both copies), and an inactivating mutation in the UL41 gene. In some embodiments, the recombinant herpes simplex virus genome comprises an inactivating mutation in the ICPO gene (one or both copies), an inactivating mutation in the ICP4 gene (one or both copies), and an inactivating mutation in the ICP22 gene. In some embodiments, the recombinant herpes simplex virus genome comprises an inactivating mutation in the ICPO gene (one or both copies), an inactivating mutation in the ICP4 gene (one or both copies), and an inactivating mutation in the UL41 gene. In some embodiments, the recombinant herpes simplex virus genome comprises an inactivating mutation in the ICPO gene (one or both copies), an inactivating mutation in the ICP22 gene, and an inactivating mutation in the UL41 gene. In some embodiments, the recombinant herpes simplex virus genome comprises an inactivating mutation in the ICPO gene (one or both copies), an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICPO (one or both copies), ICP4 (one or both copies), ICP22, and/or UL41 genes. In some embodiments, the recombinant herpes simplex virus genome further comprises an inactivating mutation in the ICP27, ICP47, and/or UL55 genes.

In some embodiments, the recombinant herpes simplex virus genome comprises an inactivating mutation in the ICP4 gene (one or both copies). In some embodiments, the recombinant herpes simplex virus genome comprises an inactivating mutation in the ICP4 gene (one or both copies) and further comprises an inactivating mutation in the ICPO (one or both copies), ICP22, ICP27, ICP47, UL41, and/or UL55 genes. In some embodiments, the recombinant herpes simplex virus genome comprises an inactivating mutation in the ICP4 gene (one or both copies), and an inactivating mutation in the ICP22 gene. In some embodiments, the recombinant herpes simplex virus genome comprises an inactivating mutation in the ICP4 gene (one or both copies), and an inactivating mutation in the UL41 gene. In some embodiments, the recombinant herpes simplex virus genome comprises an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP4 (one or both copies), ICP22, and/or UL41 genes. In some embodiments, the recombinant herpes simplex virus genome further comprises an inactivating mutation in the ICPO (one or both copies), ICP27, ICP47, and/or UL55 genes.

In some embodiments, the recombinant herpes simplex virus genome comprises an inactivating mutation in the ICP22 gene. In some embodiments, the recombinant herpes simplex virus genome comprises an inactivating mutation in the ICP22 gene and further comprises an inactivating mutation in the ICPO (one or both copies), ICP4 (one or both copies), ICP27, ICP47, UL41, and/or UL55 genes. In some embodiments, the recombinant herpes simplex virus genome comprises an inactivating mutation in the ICP22 gene, and an inactivating mutation UL41 gene. In some embodiments, the inactivating mutation is a deletion of the coding sequence of the ICP22 and/or UL41 genes. In some embodiments, the recombinant herpes simplex virus genome further comprises an inactivating mutation in the ICPO (one or both copies), ICP4 (one or both copies), ICP27, ICP47, and/or UL55 genes.

In some embodiments, the recombinant herpes simplex virus genome comprises an inactivating mutation in the ICP27 gene. In some embodiments, the recombinant herpes simplex virus genome comprises an inactivating mutation in the ICP27 gene and further comprises an inactivating mutation in the ICPO (one or both copies), ICP4 (one or both copies), ICP22, ICP47, UL41, and/or UL55 genes. In some embodiments, the inactivating mutation is a deletion of the coding sequence of the ICP27 gene.

In some embodiments, the recombinant herpes simplex virus genome comprises an inactivating mutation in the ICP47 gene. In some embodiments, the recombinant herpes simplex virus genome comprises an inactivating mutation in the ICP47 gene and further comprises an inactivating mutation in the ICPO (one or both copies), ICP4 (one or both copies), ICP22, ICP27, UL41, and/or UL55 genes. In some embodiments, the inactivating mutation is a deletion of the coding sequence of the ICP47 gene.

In some embodiments, the recombinant herpes simplex virus genome comprises an inactivating mutation in the UL41 gene. In some embodiments, the recombinant herpes simplex virus genome comprises an inactivating mutation in the UL41 gene and further comprises an inactivating mutation in the ICPO (one or both copies), ICP4 (one or both copies), ICP22, ICP27, ICP47, and/or UL55 genes. In some embodiments, the inactivating mutation is a deletion of the coding sequence of the UL41 gene.

In some embodiments, the recombinant herpes simplex virus genome comprises an inactivating mutation in the UL55 gene. In some embodiments, the recombinant herpes simplex virus genome comprises an inactivating mutation in the UL55 gene and further comprises an inactivating mutation in the ICPO (one or both copies), ICP4 (one or both copies), ICP22, ICP27, ICP47, and/or UL41 genes. In some embodiments, the inactivating mutation is a deletion of the coding sequence of the UL55 gene.

In some embodiments, the recombinant herpes simplex virus genome comprises an inactivating mutation in (e.g., a deletion of) the internal repeat (Joint) region comprising the internal repeat long (IRL) and internal repeat short (IRs) regions. In some embodiments, inactivation (e.g., deletion) of the Joint region eliminates one copy each of the ICP4 and ICPO genes. In some embodiments, inactivation (e.g., deletion) of the Joint region further inactivates (e.g., deletes) the promoter for the ICP22 and ICP47 genes. If desired, expression of one or both of these genes can be restored by insertion of an immediate early promoter into the recombinant herpes simplex virus genome (see e.g., Hill et al. (1995). Nature 375 (6530): 411-415; Goldsmith et al. (1998). J Exp Med 187 (3): 341-348). Without wishing to be bound by theory, it is believed that inactivating (e.g., deleting) the Joint region may contribute to the stability of the recombinant herpes simplex virus genome and/or allow for the recombinant herpes simplex virus genome to accommodate more and/or larger transgenes.

In some embodiments, the recombinant herpes simplex virus genome comprises an inactivating mutation in the ICP4 (one or both copies), ICP22, and ICP27 genes. In some embodiments, the recombinant herpes simplex virus genome comprises an inactivating mutation in the ICP4 (one or both copies), ICP27, and UL55 genes. In some embodiments, the recombinant herpes simplex virus genome comprises an inactivating mutation in the ICP4 (one or both copies), ICP22, ICP27, ICP47, and UL55 genes. In some embodiments, the inactivating mutation in the ICP4 (one or both copies), ICP27, and/or UL55 genes is a deletion of the coding sequence of the ICP4 (one or both copies), ICP27, and/or UL55 genes. In some embodiments, the inactivating mutation in the ICP22 and ICP47 genes is a deletion in the promoter region of the ICP22 and ICP47 genes (e.g., the ICP22 and ICP47 coding sequences are intact but are not transcriptionally active). In some embodiments, the recombinant herpes simplex virus genome comprises a deletion in the coding sequence of the ICP4 (one or both copies), ICP27, and UL55 genes, and a deletion in the promoter region of the ICP22 and ICP47 genes. In some embodiments, the recombinant herpes simplex virus genome further comprises an inactivating mutation in the ICPO (one or both copies) and/or UL41 genes.

In some embodiments, the recombinant herpes simplex virus genome comprises an inactivating mutation in the ICPO (one or both copies) gene. In some embodiments, the recombinant herpes simplex virus genome comprises an inactivating mutation in the ICPO (one or both copies) and ICP4 (one or both copies) genes. In some embodiments, the recombinant herpes simplex virus genome comprises an inactivating mutation in the ICPO (one or both copies), ICP4 (one or both copies), and ICP22 genes. In some embodiments, the recombinant herpes simplex virus genome comprises an inactivating mutation in the ICPO (one or both copies), ICP4 (one or both copies), ICP22, and ICP27 genes. In some embodiments, the recombinant herpes simplex virus genome comprises an inactivating mutation in the ICPO (one or both copies), ICP4 (one or both copies), ICP22, ICP27 and UL55 genes. In some embodiments, the inactivating mutation in the ICPO (one or both copies), ICP4 (one or both copies), ICP22, ICP27 and/or UL55 genes comprises a deletion of the coding sequence of the ICPO (one or both copies), ICP4 (one or both copies), ICP22, ICP27 and/or UL55 genes. In some embodiments, the recombinant herpes simplex virus genome further comprises an inactivating mutation in the ICP47 and/or the UL41 genes.

In some embodiments, a recombinant herpes simplex virus genome comprises one or more polynucleotides of the present disclosure within one, two, three, four, five, six, seven or more viral gene loci. Examples of suitable viral loci may include, without limitation, the ICPO (one or both copies), ICP4 (one or both copies), ICP22, ICP27, ICP47, tk, UL41 and UL55 herpes simplex viral gene loci. In some embodiments, a recombinant herpes simplex virus genome comprises one or more polynucleotides of the present disclosure within one or both of the viral ICP4 gene loci (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in one or both of the ICP4 loci). In some embodiments, a recombinant herpes simplex virus genome comprises one or more polynucleotides of the present disclosure within the viral ICP22 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the ICP22 locus). In some embodiments, a recombinant herpes simplex virus genome comprises one or more polynucleotides of the present disclosure within the viral UL41 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the UL41 locus). In some embodiments, a recombinant herpes simplex virus genome comprises one or more polynucleotides of the present disclosure within the viral ICP27 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the ICP27 locus). In some embodiments, a recombinant herpes simplex virus genome comprises one or more polynucleotides of the present disclosure within the viral ICP47 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the ICP47 locus). In some embodiments, a recombinant herpes simplex virus genome comprises one or more polynucleotides of the present disclosure within the viral UL55 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the UL55 locus). In some embodiments, a recombinant herpes simplex virus genome comprises one or more polynucleotides of the present disclosure within the viral tk gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the tk locus).

In some embodiments, a recombinant herpes simplex virus genome comprises one or more polynucleotides of the present disclosure within one or both of the viral ICP4 gene loci, and one or more polynucleotides of the present disclosure within the viral ICP22 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in one or both of the ICP4 loci, and a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the ICP22 locus). In some embodiments, a recombinant herpes simplex virus genome comprises one or more polynucleotides of the present disclosure within one or both of the viral ICP4 gene loci, and one or more polynucleotides of the present disclosure within the viral UL41 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in one or both of the ICP4 loci, and a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the UL41 locus). In some embodiments, a recombinant herpes simplex virus genome comprises one or more polynucleotides of the present disclosure within the viral ICP22 gene locus, and one or more polynucleotides of the present disclosure within the viral UL41 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the ICP22 locus, and a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the UL41 locus). In some embodiments, a recombinant herpes simplex virus genome comprises one or more polynucleotides of the present disclosure within one or both of the viral ICP4 gene loci, one or more polynucleotides of the present disclosure within the viral ICP22 gene locus, and one or more polynucleotides of the present disclosure within the viral UL41 gene locus (e.g., a recombinant virus comprising a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in one or both of the ICP4 loci, a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the ICP22 locus, and a polynucleotide encoding a polypeptide (such as an ATPase polypeptide, for example, a Calcium-transporting ATPase type 2C member 1 polypeptide or a Sarcoplasmic/endoplasmic reticulum calcium ATPase 2 polypeptide) in the UL41 locus). In some embodiments, a recombinant herpes simplex virus genome comprises one or more polynucleotides of the present disclosure within one or both of the viral ICP4 gene loci, one or more polynucleotides of the present disclosure within the viral ICP22 gene locus, one or more polynucleotides of the present disclosure within the viral UL41 gene locus, one or more polynucleotides of the present disclosure within the viral ICP27 gene locus, one or more polynucleotides of the present disclosure within the viral ICP47 gene locus, one or more polynucleotides of the present disclosure within the viral tk gene locus, and/or one or more polynucleotides of the present disclosure within the viral UL55 gene locus.

In some embodiments, the recombinant herpes virus genome (e.g., a recombinant herpes simplex virus genome) has been engineered to decrease or eliminate expression of one or more herpes virus genes (e.g., one or more toxic herpes virus genes), such as one or both copies of the HSV ICPO gene, one or both copies of the HSV ICP4 gene, the HSV ICP22 gene, the HSV UL41 gene, the HSV ICP27 gene, the HSV ICP47 gene, the HSV tk gene, the HSV UL55 gene, etc. In some embodiments, the recombinant herpes virus genome (e.g., a recombinant herpes simplex virus genome) has been engineered to reduce cytotoxicity of the recombinant genome (e.g., when introduced into a target cell), as compared to a corresponding wild-type herpes virus genome (e.g., a wild-type herpes simplex virus genome). In some embodiments, the target cell is a human cell (primary cells or a cell line derived therefrom). In some embodiments, the target cell is a cell of the epidermis and/or dermis (primary cells or a cell line derived therefrom). In some embodiments, the target cell is a keratinocyte. In some embodiments, cytotoxicity (e.g., in a target cell) of the recombinant genome (e.g., a recombinant herpes simplex virus genome) is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% as compared to a corresponding wild-type herpes virus genome (e.g., measuring the relative cytotoxicity of a recombinant ΔICP4 (one or both copies) herpes simplex virus genome vs. a wild-type herpes simplex virus genome in a target cell; measuring the relative cytotoxicity of a recombinant ΔICP4 (one or both copies)/ΔICP22 herpes simplex virus genome vs. a wild-type herpes simplex virus genome in a target cell, etc.). In some embodiments, cytotoxicity (e.g., in a target cell) of the recombinant herpes genome (e.g., a recombinant herpes simplex virus genome) is reduced by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 10⁰-fold, at least about 250-fold, at least about 500-fold, at least about 750-fold, at least about 10⁰⁰-fold, or more as compared to a corresponding wild-type herpes virus genome (e.g., measuring the relative cytotoxicity of a recombinant ΔICP4 (one or both copies) herpes simplex virus genome vs. a wild-type herpes simplex virus genome in a target cell; measuring the relative cytotoxicity of a recombinant ΔICP4 (one or both copies)/ΔICP22 herpes simplex virus genome vs. a wild-type herpes simplex virus genome in a target cell, etc.). Methods of measuring cytotoxicity are known to one of ordinary skill in the art, including, for example, through the use of vital dyes (formazan dyes), protease biomarkers, an MTT assay (or an assay using related tetrazolium salts such as XTT, MTS, water-soluble tetrazolium salts, etc.), measuring ATP content, etc.

In some embodiments, the recombinant genome (e.g., a recombinant herpes simplex virus genome) has been engineered to reduce its impact on target cell proliferation after exposure of a target cell to the recombinant genome, as compared to a corresponding wild-type genome (e.g., a wild-type herpes simplex virus genome). In some embodiments, the target cell is a human cell (primary cells or a cell line derived therefrom). In some embodiments, the target cell is a cell of the epidermis and/or dermis (primary cells or a cell line derived therefrom). In some embodiments, the target cell is a keratinocyte. In some embodiments, target cell proliferation after exposure to the recombinant genome is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% faster as compared to target cell proliferation after exposure to a corresponding wild-type genome (e.g., measuring the relative cellular proliferation after exposure to a recombinant ΔICP4 (one or both copies) herpes simplex virus genome vs. cellular proliferation after exposure to a wild-type herpes simplex virus genome in target cells; measuring the relative cellular proliferation after exposure to a recombinant ΔICP4 (one or both copies)/ΔICP22 herpes simplex virus genome vs. cellular proliferation after exposure to a wild-type herpes simplex virus genome in target cells, etc.). In some embodiments, target cell proliferation after exposure to the recombinant genome is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 10⁰-fold, at least about 250-fold, at least about 500-fold, at least about 750-fold, or at least about 10⁰⁰-fold faster as compared to target cell proliferation after exposure to a corresponding wild-type genome (e.g., measuring the relative cellular proliferation after exposure to a recombinant ΔICP4 (one or both copies) herpes simplex virus genome vs. cellular proliferation after exposure to a wild-type herpes simplex virus genome in target cells; measuring the relative cellular proliferation after exposure to a recombinant ΔICP4 (one or both copies)/ΔICP22 herpes simplex virus genome vs. cellular proliferation after exposure to a wild-type herpes simplex virus genome in target cells, etc.). Methods of measuring cellular proliferation are known to one of ordinary skill in the art, including, for example, through the use of a Ki67 cell proliferation assay, a BrdU cell proliferation assay, etc.

A vector (e.g., herpes viral vector) may include one or more polynucleotides of the present disclosure in a form suitable for expression of the polynucleotide in a host cell. Vectors may include one or more regulatory sequences operatively linked to the polynucleotide to be expressed (e.g., as described above).

In some embodiments, the present disclosure relates to one or more heterologous polynucleotides (e.g., a bacterial artificial chromosome (BAC)) comprising any of the recombinant nucleic acids described herein.

In some embodiments, a recombinant nucleic acid (e.g., a recombinant herpes simplex virus genome) of the present disclosure comprises one or more of the polynucleotides described herein inserted in any orientation in the recombinant nucleic acid. If the recombinant nucleic acid comprises two or more polynucleotides described herein (e.g., two or more, three or more, etc.), the polynucleotides may be inserted in the same orientation or opposite orientations to one another. Without wishing to be bound by theory, incorporating two polynucleotides (e.g., two transgenes) into a recombinant nucleic acid (e.g., a vector) in an antisense orientation may help to avoid read-through and ensure proper expression of each polynucleotide.

In some embodiments, a recombinant nucleic of the present disclosure does not comprise a polynucleotide encoding a Collagen alpha-1 (VII) chain polypeptide (COL7). In some embodiments, a recombinant nucleic of the present disclosure does not comprise a polynucleotide encoding a Lysyl hydroxylase 3 polypeptide (LH3). In some embodiments, a recombinant nucleic of the present disclosure does not comprise a polynucleotide encoding a Keratin type I cytoskeletal 17 polypeptide (KRT17). In some embodiments, a recombinant nucleic of the present disclosure does not comprise a polynucleotide encoding a transglutaminase (TGM) polypeptide (e.g., a human transglutaminase polypeptide such as a human TGM1 polypeptide and/or a human TGM5 polypeptide). In some embodiments, a recombinant nucleic of the present disclosure does not comprise a polynucleotide encoding a cosmetic protein (e.g., collagen proteins, fibronectins, elastins, lumicans, vitronectins/vitronectin receptors, laminins, neuromodulators, fibrillins, additional dermal extracellular matrix proteins, etc.). In some embodiments, a recombinant nucleic of the present disclosure does not comprise a polynucleotide encoding an antibody (e.g., a full-length antibody, an antibody fragment, etc.). In some embodiments, a recombinant nucleic of the present disclosure does not comprise a polynucleotide encoding a Serine Protease Inhibitor Kazal-type (SPINK) polypeptide (e.g., a human SPINK polypeptide, such as a SPINK5 polypeptide). In some embodiments, a recombinant nucleic of the present disclosure does not comprise a polynucleotide encoding a filaggrin or filaggrin 2 polypeptide (e.g., a human filaggrin or filaggrin 2 polypeptide). In some embodiments, a recombinant nucleic of the present disclosure does not comprise a polynucleotide encoding a Cystic Fibrosis Transmembrane Conductance Regulator (CFTR) polypeptide (e.g., a human CFTR polypeptide). In some embodiments, a recombinant nucleic of the present disclosure does not comprise a polynucleotide encoding an ichthyosis-associated polypeptide (e.g., an ATP-binding cassette sub-family A member 12 polypeptide, a 1-acylglycerol-3-phosphate O-acyltransferase ABHD5 polypeptide, an Aldehyde dehydrogenase family 3 member A2 polypeptide, an Arachidonate 12-lipoxygenase 12R-type polypeptide, a Hydroperoxide isomerase ALOXE3 polypeptide, an AP-1 complex subunit sigma-1A polypeptide, an Arylsulfatase E polypeptide, a Caspase-14 polypeptide, a Corneodesmosin polypeptide, a Ceramide synthase 3 polypeptide, a Carbohydrate sulfotransferase 8 polypeptide, a Claudin-1 polypeptide, a Cystatin-A polypeptide, a Cytochrome P450 4F22 polypeptide, a 3-beta-hydroxysteroid-Delta (8),Delta (7)-isomerase polypeptide, an Elongation of very long chain fatty acids protein 4 polypeptide, a Filaggrin polypeptide, a Filaggrin 2 polypeptide, a Gap junction beta-2 polypeptide, a Gap junction beta-3 polypeptide, a Gap junction beta-4 polypeptide, a Gap junction beta-6 polypeptide, a 3-ketodihydrosphingosine reductase polypeptide, a Keratin, type II cytoskeletal 1 polypeptide, a Keratin, type II cytoskeletal 2 epidermal polypeptide, a Keratin, type I cytoskeletal 9 polypeptide, a Keratin, type I cytoskeletal 10 polypeptide, a Lipase member N polypeptide, a Loricrin polypeptide, a Membrane-bound transcription factor site-2 protease polypeptide, a Magnesium transporter NIPA4 polypeptide, a Sterol-4-alpha-carboxylate 3-dehydrogenase, decarboxylating polypeptide, a Peroxisomal targeting signal 2 receptor polypeptide, a D-3-phosphoglycerate dehydrogenase polypeptide, a Phytanoyl-CoA dioxygenase, peroxisomal polypeptide, Patatin-like phospholipase domain-containing protein 1 polypeptide, a Proteasome maturation protein polypeptide, a Phosphoserine aminotransferase polypeptide, a Short-chain dehydrogenase/reductase family 9C member 7 polypeptide, a Serpin B8 polypeptide, a Long-chain fatty acid transport protein 4 polypeptide, a Synaptosomal-associated protein 29 polypeptide, a Suppressor of tumorigenicity 14 protein polypeptide, a Steryl-sulfatase polypeptide, a Vacuolar protein sorting-associated protein 33B polypeptide, and a CAAX prenyl protease 1 homolog polypeptide). In some embodiments, a recombinant nucleic of the present disclosure does not comprise a polynucleotide encoding a Collagen alpha-1 (VII) chain polypeptide, a Lysyl hydroxylase 3 polypeptide, a Keratin type I cytoskeletal 17 polypeptide, and/or any chimeric polypeptides thereof. In some embodiments, a recombinant nucleic of the present disclosure does not comprise a polynucleotide encoding a Collagen alpha-1 (VII) chain polypeptide, a Lysyl hydroxylase 3 polypeptide, a Keratin type I cytoskeletal 17 polypeptide, a transglutaminase (TGM) polypeptide, a filaggrin polypeptide, a cosmetic protein, an antibody, a SPINK polypeptide, a CFTR polypeptide, an ichthyosis-associated polypeptide, an Alpha-1-antitrypsin polypeptide, a Sodium-dependent phosphate transport protein 2B polypeptide, a Dynein heavy chain 5 axonemal polypeptide, a Dynein heavy chain 11 axonemal polypeptide, a Coiled-coil domain-containing protein 39 polypeptide, a Dynein intermediate chain 1 axonemal polypeptide, a Coiled-coil domain-containing protein 40 polypeptide, a Coiled-coil domain containing protein 103 polypeptide, a Sperm-associated antigen 1 polypeptide, a Zinc finger MYND domain-containing protein 10 polypeptide, an Armadillo repeat containing protein 4 polypeptide, a Coiled-coil domain-containing protein 151 polypeptide, a Dynein intermediate chain 2 axonemal polypeptide, a Radial spoke head 1 homolog polypeptide, a Coiled-coil domain-containing protein 114 polypeptide, a Radial spoke head protein 4 homolog A polypeptide, a Dynein assembly factor 1 axonemal polypeptide, a Dynein assembly factor 2 axonemal polypeptide, a Leucine-rich repeat-containing protein 6 polypeptide, a Pulmonary surfactant-associated protein B polypeptide, a Pulmonary surfactant-associated protein C polypeptide, a Homeobox protein Nkx-2.1 polypeptide, an ATP-binding cassette sub-family A member 3 polypeptide, a Cytokine receptor common subunit beta polypeptide, a Granulocyte-macrophage colony-stimulating factor receptor subunit alpha polypeptide, a Bone morphogenetic protein receptor type-2 polypeptide, a Sarcoplasmic/endoplasmic reticulum calcium ATPase 2 polypeptide, a serine/threonine-protein kinase receptor R3 polypeptide, an Endoglin polypeptide, a Mothers against decapentaplegic homolog 9 polypeptide, a Caveolin-1 polypeptide, a Potassium channel subfamily K member 3 polypeptide, an eIF-2-alpha kinase GCN2 polypeptide, a Pulmonary surfactant-associated protein A2 polypeptide, a Telomerase reverse transcriptase polypeptide, a Dyskerin polypeptide, a Regulator of telomere elongation helicase 1 polypeptide, a Poly (A)-specific ribonuclease PARN polypeptide, a TERF1-interacting nuclear factor 2 polypeptide, an H/ACA ribonucleoprotein complex non-core subunit NAF1 polypeptide, a Mucin-5B polypeptide, a Desmoplakin polypeptide, a CST complex subunit STN1 polypeptide, a Dipeptidyl peptidase 9 polypeptide, and/or any chimeric polypeptides thereof.

In some embodiments, a recombinant nucleic acid of the present disclosure does not comprise a polynucleotide encoding an immunomodulatory polypeptide. In some embodiments, a recombinant nucleic acid of the present disclosure does not comprise a polynucleotide encoding a cytokine polypeptide and/or a chemokine polypeptide. In some embodiments, a recombinant nucleic acid of the present disclosure does not comprise a polynucleotide encoding a cytokine polypeptide. In some embodiments, a recombinant nucleic acid of the present disclosure does not comprise a polynucleotide encoding an IL-1 polypeptide, an IL-2 polypeptide, an IL-7 polypeptide, an IL-12 polypeptide, an IL-13 polypeptide, an IL-15 polypeptide, an IL-17 polypeptide, an IL-18 polypeptide, an IL-28 polypeptide, an IL-32 polypeptide, an IL-33 polypeptide, an IL-34 polypeptide, a TNFα polypeptide, an IFNγ polypeptide, a G-CSF polypeptide, a GM-CSF polypeptide, and/or any chimeric polypeptides thereof.

In some embodiments, a recombinant nucleic acid of the present disclosure does not comprise a polynucleotide encoding a chemokine polypeptide. In some embodiments, a recombinant nucleic acid of the present disclosure does not comprise a polynucleotide encoding a CXCL1 polypeptide, a CXCL2 polypeptide, a CXCL8 polypeptide, a CXCL9 polypeptide, a CXCL11 polypeptide, a CXCL16 polypeptide, a CCL2 polypeptide, a CCL3 polypeptide, a CCL4 polypeptide, a CCL5 polypeptide, a CCL11 polypeptide, and/or any chimeric polypeptides thereof.

In some embodiments, a recombinant nucleic acid of the present disclosure does not comprise a polynucleotide encoding a sterile alpha motif domain-containing protein 11 polypeptide, a nephrocystin-4 polypeptide, an espin polypeptide, a nicotinamide/nicotinic acid mononucleotide adenylyltransferase 1 polypeptide, a mitofusin-2 polypeptide, an ER membrane protein complex subunit 1 polypeptide, a phospholipase A2 group V polypeptide, a dehydrodolichyl diphosphate synthase complex subunit polypeptide, a palmitoyl-protein thioesterase 1 polypeptide, an elongation of very long chain fatty acids protein 1 polypeptide, a protein O-linked-mannose beta-1,2-N-acetylglucosaminyltransferase 1 polypeptide, a retinoid isomerohydrolase polypeptide, a retinal-specific phospholipid-transporting ATPase ABCA4 polypeptide, a Collagen alpha-1 (XI) polypeptide, a Guanine nucleotide-binding protein G (t) subunit alpha-2 polypeptide, a Chloride channel CLIC-like protein 1 polypeptide, a DNA damage-regulated autophagy modulator protein 2 polypeptide, an U4/U6 small nuclear ribonucleoprotein Prp3 polypeptide, an Alpha-endosulfine polypeptide, a Semaphorin-4A polypeptide, a Cyclic AMP-dependent transcription factor ATF-6 alpha polypeptide, a Hemicentin-1 polypeptide, a Complement factor H polypeptide, a Protein crumbs homolog 1 polypeptide, an Adiponectin receptor protein 1 polypeptide, a Protein RD3 polypeptide, a Serine/threonine-protein kinase Nek2 polypeptide, a Feline leukemia virus subgroup C receptor-related protein 1 polypeptide, an Usherin polypeptide, a Serologically defined colon cancer antigen 8 polypeptide, an Olfactory receptor 2W3 polypeptide, a NBAS subunit of NRZ tethering complex polypeptide, a Cytosolic carboxypeptidase-like protein 5 polypeptide, a Zinc finger protein 513 polypeptide, an Intraflagellar transport protein 172 homolog polypeptide, a Photoreceptor cilium actin regulator polypeptide, an EGF-containing fibulin-like extracellular matrix protein 1/TLE family member 5 polypeptide, a Protein FAM161A polypeptide, a WD repeat-containing and planar cell polarity effector protein fritz homolog polypeptide, a Centrosome-associated protein ALMS1 polypeptide, an U5 small nuclear ribonucleoprotein 200 kDa helicase polypeptide, a Metal transporter CNNM4 polypeptide, a Cyclic nucleotide-gated cation channel alpha-3 polypeptide, a Nephrocystin-1 polypeptide, a Tyrosine-protein kinase Mer polypeptide, a Bardet-Biedl syndrome 5 protein polypeptide, a Ceramide kinase-like protein polypeptide, a Neurogenic differentiation factor 1 polypeptide, a Transmembrane protein 237 polypeptide, an Inward rectifier potassium channel 13 polypeptide, a S-arrestin polypeptide, a Secreted phosphoprotein 24 polypeptide, a CCA tRNA nucleotidyltransferase 1 polypeptide, a mitochondrial, Sodium bicarbonate cotransporter 3 polypeptide, a Leucine zipper transcription factor-like protein 1 polypeptide, a Guanine nucleotide-binding protein G (t) subunit alpha-1 polypeptide, a Three-prime repair exonuclease 1 polypeptide, a MAP kinase-activated protein kinase 3 polypeptide, an Ataxin-7 polypeptide, a Vitamin K-dependent protein S polypeptide, an ADP-ribosylation factor-like protein 6 polypeptide, an Interphotoreceptor matrix proteoglycan 2 polypeptide, an IQ calmodulin-binding motif-containing protein 1 polypeptide, a Rhodopsin polypeptide, a Nephrocystin-3 polypeptide, a Clarin-1 polypeptide, a Probable cationic amino acid transporter/Solute carrier family 7 member 14 polypeptide, a Choline-phosphate cytidylyltransferase A polypeptide, a Centrosomal protein of 19 kDa polypeptide, a Rod cGMP-specific 3′,5′-cyclic phosphodiesterase subunit beta polypeptide, a Wolframin, Homeobox protein HMX1 polypeptide, a Ras-related protein Rab-28 polypeptide, a Coiled-coil and C2 domain-containing protein 2A polypeptide, a Prominin-1 polypeptide, an Adhesion G protein-coupled receptor A3 polypeptide, a Death domain-containing protein 1 polypeptide, a WD repeat-containing protein 19 polypeptide, a cGMP-gated cation channel alpha-1 polypeptide, a CDGSH iron-sulfur domain-containing protein 2 polypeptide, a Microsomal triglyceride transfer protein large subunit polypeptide, a Leucine-rich repeat, immunoglobulin-like domain and transmembrane domain-containing protein 3 polypeptide, a Bardet-Biedl syndrome 7 protein polypeptide, a Bardet-Biedl syndrome 12 protein polypeptide, a Major facilitator superfamily domain-containing protein 8 polypeptide, a Serine/threonine-protein kinase PLK4 polypeptide, a Lecithin retinol acyltransferase polypeptide, a Toll-like receptor 3 polypeptide, a Cytochrome P450 4V2 polypeptide, a Spliceosome-associated protein CWC27 homolog polypeptide, a Centrosomal protein POC5 polypeptide, a Versican core protein polypeptide, an Adhesion G-protein coupled receptor V1 polypeptide, a COUP transcription factor 1 polypeptide, a Mitochondrial outer membrane protein SLC25A46 polypeptide, a Catenin alpha-1 polypeptide, a Histidine-tRNA ligase, cytoplasmic polypeptide, a Rod cGMP-specific 3′,5′-cyclic phosphodiesterase subunit alpha polypeptide, a Metabotropic glutamate receptor 6 polypeptide, a Serine/threonine-protein kinase MAK polypeptide, a Complement C2 polypeptide, a Complement factor B polypeptide, a Tubby-related protein 1 polypeptide, a Guanylyl cyclase-activating protein 1 polypeptide, a Guanylyl cyclase-activating protein 2 polypeptide, a Peripherin-2 polypeptide, an Interphotoreceptor matrix proteoglycan 1 polypeptide, a Protein eyes shut homolog polypeptide, a Collagen alpha-1 (IX) chain polypeptide, a Regulating synaptic membrane exocytosis protein 1 polypeptide, a Lebercilin polypeptide, an Elongation of very long chain fatty acids protein 4 polypeptide, a PR domain zinc finger protein 13 polypeptide, a Reticulon-4-interacting protein 1, mitochondrial, polypeptide, a Jouberin polypeptide, a Peroxisomal targeting signal 2 receptor polypeptide, a CCR4-NOT transcription complex subunit 9 polypeptide, an Aryl hydrocarbon receptor polypeptide, a Kelch-like protein 7 polypeptide, a Retinitis pigmentosa 9 protein polypeptide, a Protein PTHB1 polypeptide, a Peroxisomal ATPase PEX1 polypeptide, a Tetraspanin-12 polypeptide, an Inosine-5′-monophosphate dehydrogenase 1 polypeptide, a Short-wave-sensitive opsin 1 polypeptide, an UPF0606 protein KIAA1549 polypeptide, a Retinitis pigmentosa 1-like 1 protein polypeptide, a Disintegrin and metalloproteinase domain-containing protein 9 polypeptide, a Heparan-alpha-glucosaminide N-acetyltransferase polypeptide, an Oxygen-regulated protein 1 polypeptide, an Alpha-tocopherol transfer protein polypeptide, a Centrosome and spindle pole-associated protein 1 polypeptide, a Dynamin-like 120 kDa protein, mitochondrial, polypeptide, a Peroxisome biogenesis factor 2 polypeptide, a Cyclic nucleotide-gated cation channel beta-3 polypeptide, a Cilia- and flagella-associated protein 418 polypeptide, a Growth/differentiation factor 6 polypeptide, a Regulating synaptic membrane exocytosis protein 2 polypeptide, a Potassium voltage-gated channel subfamily V member 2 polypeptide, an E3 ubiquitin-protein ligase Topors polypeptide, a Centrosomal protein of 78 kDa polypeptide, an Inversin polypeptide, an U4/U6 small nuclear ribonucleoprotein Prp4 polypeptide, a Whirlin polypeptide, an E3 ubiquitin-protein ligase TRIM32 polypeptide, a Toll-like receptor 4 polypeptide, a Cytoplasmic dynein 2 intermediate chain 2 polypeptide, a Programmed cell death protein 2 polypeptide, an Exosome complex component RRP4 polypeptide, a Phosphatidylinositol polyphosphate 5-phosphatase type IV polypeptide, a Phytanoyl-CoA dioxygenase, peroxisomal, polypeptide, an Acyl-CoA-binding domain-containing protein polypeptide, a Protocadherin-15 polypeptide, a Retinol-binding protein 3 polypeptide, a DNA excision repair protein ERCC-6 polypeptide, a Hexokinase-1 polypeptide, a Cadherin-23 polypeptide, a Cadherin-related family member 1 polypeptide, a RPE-retinal G protein-coupled receptor polypeptide, a Kinesin-like protein KIF11 polypeptide, a Retinol-binding protein 4 polypeptide, a Cone cGMP-specific 3′,5′-cyclic phosphodiesterase subunit alpha polypeptide, a Paired box protein Pax-2 polypeptide, a PDZ domain-containing protein 7 polypeptide, an ADP-ribosylation factor-like protein 3 polypeptide, a BBSome-interacting protein 1 polypeptide, an Age-related maculopathy susceptibility protein 2 polypeptide, a Serine protease HTRA1 polypeptide, an Ornithine aminotransferase, mitochondrial, polypeptide, a Zinc finger protein 408 polypeptide, a Tubby protein homolog polypeptide, a Transcriptional enhancer factor TEF-1 polypeptide, a Harmonin polypeptide, a Transmembrane protein 216 polypeptide, a Bestrophin-1 polypeptide, an Isoaspartyl peptidase/L-asparaginase polypeptide, a Rod outer segment membrane protein 1 polypeptide, a Bardet-Biedl syndrome 1 protein polypeptide, a Calcium-binding protein 4 polypeptide, a Low-density lipoprotein receptor-related protein 5 polypeptide, a Calpain-5 polypeptide, an Unconventional myosin-VIIa polypeptide, a Transmembrane protein 126A polypeptide, a Frizzled-4 polypeptide, a Cytoplasmic dynein 2 light intermediate chain 1 polypeptide, a Centrosomal protein of 164 kDa polypeptide, a Complement Clq tumor necrosis factor-related protein 5 polypeptide, a Membrane frizzled-related protein polypeptide, a Voltage-dependent calcium channel subunit alpha-2/delta-4 polypeptide, a Guanine nucleotide-binding protein G (I)/G(S)/G (T) subunit beta-3 polypeptide, a Retinal cone rhodopsin-sensitive cGMP 3′,5′-cyclic phosphodiesterase subunit gamma polypeptide, a Matrix metalloproteinase-19 polypeptide, a Retinol dehydrogenase 5 polypeptide, a T-complex protein 1 subunit beta polypeptide, a Bardet-Biedl syndrome 10 protein polypeptide, a Centrosomal protein of 290 kDa polypeptide, a POC1 centriolar protein homolog B polypeptide, a Mevalonate kinase polypeptide, an Intraflagellar transport protein 81 homolog polypeptide, a Mitochondrial translation release factor in rescue polypeptide, an Integral membrane protein 2B polypeptide, a Retinoblastoma-associated protein polypeptide, a RCC1 and BTB domain-containing protein 1 polypeptide, a Rhodopsin kinase GRK1 polypeptide, an X-linked retinitis pigmentosa GTPase regulator-interacting protein 1 polypeptide, a Neural retina-specific leucine zipper protein polypeptide, a Homeobox protein OTX2 polypeptide, a Retinol dehydrogenase 11 polypeptide, a Retinol dehydrogenase 12 polypeptide, a Tubulin polyglutamylase TTLL5 polypeptide, a Spermatogenesis-associated protein 7 polypeptide, a Tetratricopeptide repeat protein 8 polypeptide, a Fibulin-5 polypeptide, a Transient receptor potential cation channel subfamily M member 1 polypeptide, a Gamma-tubulin complex component 4 polypeptide, a Sodium/potassium/calcium exchanger 1 polypeptide, a Photoreceptor-specific nuclear receptor polypeptide, a Bardet-Biedl syndrome 4 protein polypeptide, a Calcium and integrin-binding family member 2 polypeptide, a Retinaldehyde-binding protein 1 polypeptide, a N-acetylglucosamine-1-phosphotransferase subunit gamma polypeptide, an Intraflagellar transport protein 140 homolog polypeptide, a Clusterin-associated protein 1 polypeptide, an ATP-binding cassette sub-family C member 6 polypeptide, a Ketimine reductase mu-crystallin polypeptide, a Battenin polypeptide, a Zinc finger protein 423 polypeptide, a Protein fantom polypeptide, a Bardet-Biedl syndrome 2 protein polypeptide, an ADP-ribosylation factor-like protein 2-binding protein polypeptide, a Cyclic nucleotide-gated cation channel beta-1 polypeptide, a Cadherin-3 polypeptide, a Pre-mRNA-splicing factor ATP-dependent RNA helicase PRP16 polypeptide, a disintegrin and metalloproteinase with thrombospondin motifs 18 polypeptide, a Solute carrier family 38 member 8 polypeptide, a Retinal guanylyl cyclase 1 polypeptide, a Pre-mRNA-processing-splicing factor 8 polypeptide, an Aryl-hydrocarbon-interacting protein-like 1 polypeptide, a Membrane-associated phosphatidylinositol transfer protein 3 polypeptide, a Protein unc-119 homolog A polypeptide, a Probable G-protein coupled receptor 179 polypeptide, a Tectonic-like complex member MKS1 polypeptide, a Carbonic anhydrase 4 polypeptide, a Regulator of G-protein signaling 9 polypeptide, an Arylsulfatase G polypeptide, a pre-mRNA splicing regulator USH1G polypeptide, a Photoreceptor disk component PRCD polypeptide, a Fascin-2 polypeptide, a Retinal rod rhodopsin-sensitive cGMP 3′,5′-cyclic phosphodiesterase subunit gamma polypeptide, a Laminin subunit alpha-1 polypeptide, an AFG3-like protein 2 polypeptide, a Cone-rod homeobox protein polypeptide, a Receptor expression-enhancing protein 6 polypeptide, a Retina and anterior neural fold homeobox protein 2 polypeptide, a Complement C3 polypeptide, a Rho guanine nucleotide exchange factor 18 polypeptide, a Patatin-like phospholipase domain-containing protein 6 polypeptide, a Regulator of G-protein signaling 9-binding protein polypeptide, an Optic atrophy 3 protein polypeptide, an U4/U6 small nuclear ribonucleoprotein Prp31 polypeptide, an Isocitrate dehydrogenase [NAD] subunit beta, mitochondrial, polypeptide, a Pantothenate kinase 2, mitochondrial, polypeptide, a Protein jagged-1 polypeptide, a Molecular chaperone MKKS polypeptide, a Centrosomal protein kizuna, Lysophosphatidylserine lipase ABHD12 polypeptide, a Kinesin-like protein KIF3B polypeptide, a Centrosome-associated protein CEP250 polypeptide, a Pre-mRNA-processing factor 6 polypeptide, a Cilia- and flagella-associated protein 410 polypeptide, a Dynamin-1-like protein polypeptide, a Metalloproteinase inhibitor 3 polypeptide, an Intraflagellar transport protein 27 homolog polypeptide, a Fibulin-1 polypeptide, a MIEF1 upstream open reading frame protein polypeptide, an Aconitate hydratase, mitochondrial, polypeptide, a Gamma-tubulin complex component 6 polypeptide, a Centriole and centriolar satellite protein polypeptide, a Retinoschisin, Protein XRP2 polypeptide, a Dystrophin polypeptide, an X-linked retinitis pigmentosa GTPase regulator polypeptide, a Nyctalopin polypeptide, an Xaa-Pro dipeptidase polypeptide, a Norrin polypeptide, a Voltage-dependent L-type calcium channel subunit alpha-1F polypeptide, a Phosphoglycerate kinase 1 polypeptide, a Rab proteins geranylgeranyltransferase component A 1 polypeptide, a Mitochondrial import inner membrane translocase subunit Tim8 A polypeptide, a Ribose-phosphate pyrophosphokinase 1 polypeptide, a Long-wave-sensitive opsin 1 polypeptide, a Medium-wave-sensitive opsin 1 polypeptide, a Short-wave-sensitive opsin 1 polypeptide, a Transcription factor A, mitochondrial, polypeptide, a NADH-ubiquinone oxidoreductase chain 1 polypeptide, a NADH-ubiquinone oxidoreductase chain 2 polypeptide, a NADH-ubiquinone oxidoreductase chain 3 polypeptide, a NADH-ubiquinone oxidoreductase chain 4L polypeptide, a NADH-ubiquinone oxidoreductase chain 4 polypeptide, a NADH-ubiquinone oxidoreductase chain 5 polypeptide, a NADH-ubiquinone oxidoreductase chain 6 polypeptide, an ATP synthase subunit a polypeptide, an ATP synthase protein 8 polypeptide, a Cytochrome c oxidase subunit 1 polypeptide, a Cytochrome c oxidase subunit 3 polypeptide, a Cytochrome b polypeptide, a Leucine-tRNA ligase, mitochondrial, polypeptide, a Nondiscriminating glutamyl-tRNA synthetase EARS2, mitochondrial, polypeptide, a Lysine-tRNA ligase polypeptide, a Histidine-tRNA ligase, mitochondrial, polypeptide, aSerine-tRNA ligase, mitochondrial, polypeptide, a Probable proline-tRNA ligase, mitochondrial, polypeptide, a Cyanocobalamin reductase/alkylcobalamin dealkylase polypeptide, a POU domain, class 3, transcription factor 4 polypeptide, a Ribosomal protein S6 kinase alpha-6 polypeptide, a Ciliogenesis and planar polarity effector 1 polypeptide, a Meckelin, TRAF3-interacting protein 1 polypeptide, an Intraflagellar transport protein 74 homolog polypeptide, an S phase cyclin A-associated protein in the endoplasmic reticulum polypeptide, a Sodium channel and clathrin linker 1 polypeptide, a Protein TALPID3 polypeptide, a Tectonic-2 polypeptide, an ADP-ribosylation factor-like protein 13B polypeptide, a B9 domain-containing protein 1 polypeptide, a B9 domain-containing protein 2 polypeptide, a C2 domain-containing protein 3 polypeptide, a Centrosomal protein of 41 kDa polypeptide, a Centrosomal protein of 104 kDa polypeptide, a Centrosomal protein of 120 kDa polypeptide, an Intraflagellar transport protein 172 homolog polypeptide, a Katanin-interacting protein polypeptide, a Kinesin-like protein KIF7 polypeptide, a Retinal rod rhodopsin-sensitive cGMP 3′,5′-cyclic phosphodiesterase subunit delta polypeptide, a Tectonic-1 polypeptide, a Tectonic-3 polypeptide, a Transmembrane protein 107 polypeptide, a Transmembrane protein 138 polypeptide, a Transmembrane protein 231 polypeptide, a Tetratricopeptide repeat protein 21B polypeptide, a Nuclear receptor ROR-alpha polypeptide, a Beta-nerve growth factor polypeptide, a Solute carrier family 4 member 11 polypeptide, a Zinc finger E-box-binding homeobox 1 polypeptide, a Keratin, type II cuticular Hb3 polypeptide, a Keratin, type I cytoskeletal 12 polypeptide, a Transforming growth factor-beta-induced protein ig-h3 polypeptide, a Tumor-associated calcium signal transducer 2 polypeptide, a Carbohydrate sulfotransferase 6 polypeptide, a Gelsolin polypeptide, an UbiA prenyltransferase domain-containing protein 1 polypeptide, a Decorin polypeptide, a 1-phosphatidylinositol 3-phosphate 5-kinase polypeptide, a Transcription factor Ovo-like 2 polypeptide, a Grainyhead-like protein 2 homolog polypeptide, and/or any chimeric polypeptides thereof.

### IV. Viruses

Certain aspects of the present disclosure relate to viruses comprising any of the polynucleotides and/or recombinant nucleic acids described herein. In some embodiments, the virus is capable of infecting one or more target cells of a subject (e.g., a human). In some embodiments, the virus is suitable for delivering the polynucleotides and/or recombinant nucleic acids into one or more target cells of a subject (e.g., a human). In some embodiments, the present disclosure relates to one or more viral particles comprising any of the polynucleotides and/or recombinant nucleic acids described herein. In some embodiments, the one or more target cells are one or more human cells. In some embodiments, the one or more target cells are one or more cells with an ATPase deficiency (e.g., one or more cells comprising a loss-of-function mutation in, or a pathogenic variant of, a native gene such as an ATP2C1 gene or an ATP2A2 gene). In some embodiments, the one or more target cells are one or more cells of the mucosa. In some embodiments, the one or more target cells are one or more cells of the skin (e.g., one or more cells of the epidermis, dermis, and/or subcutis). In some embodiments, the one or more target cells are cells of the epidermis and/or dermis (e.g., cells of the human epidermis and/or dermis). In some embodiments, the one or more target cells are selected from keratinocytes, melanocytes, Langerhans cells, Merkel cells, mast cells, fibroblasts, and/or adipocytes. In some embodiments, the one or more target cells are keratinocytes. In some embodiments, the one or more target cells reside in the stratum corneum, stratum granulosum, stratum spinulosum, stratum basale, and/or basement membrane. In some embodiments, the one or more target cells are one or more epidermal cells. In some embodiments, the one or more target cells are one or more dermal cells.

Any suitable virus known in the art may be used, including, for example, adenovirus, adeno-associated virus, retrovirus, lentivirus, sendai virus, papillomavirus, herpes virus (e.g., a herpes simplex virus), vaccinia virus, and/or any hybrid or derivative viruses thereof. In some embodiments, the virus is attenuated. In some embodiments, the virus is replication competent. In some embodiments, the virus is replication defective. In some embodiments, the virus is not oncolytic. In some embodiments, the virus has been modified to alter its tissue tropism relative to the tissue tropism of a corresponding unmodified, wild-type virus. In some embodiments, the virus has reduced cytotoxicity (e.g., in a target cell) as compared to a corresponding wild-type virus. Methods of producing a virus comprising recombinant nucleic acids are well known to one of ordinary skill in the art.

In some embodiments, the virus is a member of the Herpesviridae family of DNA viruses, including, for example, a herpes simplex virus, a varicella zoster virus, a human cytomegalovirus, a herpesvirus 6A, a herpesvirus 6B, a herpesvirus 7, an Epstein-Barr virus, and a Kaposi's sarcoma-associated herpesvirus, etc. In some embodiments, the herpes virus is attenuated. In some embodiments, the herpes virus is replication defective. In some embodiments, the herpes virus is replication competent. In some embodiments, the herpes virus has been engineered to reduce or eliminate expression of one or more herpes virus genes (e.g., one or more toxic herpes virus genes). In some embodiments, the herpes virus has reduced cytotoxicity as compared to a corresponding wild-type herpes virus. In some embodiments, the herpes virus is not oncolytic.

In some embodiments, the virus is a herpes simplex virus. Herpes simplex viruses comprising recombinant nucleic acids may be produced by a process disclosed, for example, in WO2015/009952, WO2017/176336, WO2019/200163, and/or WO2019/210219. In some embodiments, the herpes simplex virus is attenuated. In some embodiments, the herpes simplex virus is replication defective. In some embodiments, the herpes simplex virus is replication competent. In some embodiments, the herpes simplex virus has been engineered to reduce or eliminate expression of one or more herpes simplex virus genes (e.g., one or more toxic herpes simplex virus genes). In some embodiments, the herpes simplex virus has reduced cytotoxicity as compared to a corresponding wild-type herpes simplex virus. In some embodiments, the herpes simplex virus is not oncolytic. In some embodiments, the herpes simplex virus is an HSV-1, an HSV-2, or any derivatives thereof. In some embodiments, the herpes simplex virus is an HSV-1 virus. In some embodiments, the HSV-1 is attenuated. In some embodiments, the HSV-1 is replication defective. In some embodiments, the HSV-1 is replication competent. In some embodiments, the HSV-1 has been engineered to reduce or eliminate expression of one or more HSV-1 genes (e.g., one or more toxic HSV-1 genes). In some embodiments, the HSV-1 has reduced cytotoxicity as compared to a corresponding wild-type HSV-1. In some embodiments, the HSV-1 is not oncolytic.

In some embodiments, the herpes simplex virus has been modified to alter its tissue tropism relative to the tissue tropism of an unmodified, wild-type herpes simplex virus. In some embodiments, the herpes simplex virus comprises a modified envelope. In some embodiments, the modified envelope comprises one or more (e.g., one or more, two or more, three or more, four or more, etc.) mutant herpes simplex virus glycoproteins. Examples of herpes simplex virus glycoproteins may include, but are not limited to, the glycoproteins gB, gC, gD, gH, and gL. In some embodiments, the modified envelope alters the herpes simplex virus tissue tropism relative to a wild-type herpes simplex virus.

In some embodiments, the transduction efficiency (in vitro and/or in vivo) of a virus of the present disclosure (e.g., a herpes virus such as a herpes simplex virus) for one or more target cells (e.g., one or more cells of the epidermis and/or dermis) is at least about 25%. For example, the transduction efficiency of the virus for one or more target cells may be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 99.5%, or more. In some embodiments, the virus is a herpes simplex virus and the transduction efficiency of the virus for one or more target cells (e.g., one or more cells of the epidermis and/or dermis) is about 85% to about 10⁰%. In some embodiments, the virus is a herpes simplex virus and the transduction efficiency of the virus for one or more target cells (e.g., one or more cells of the epidermis and/or dermis) is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100%. Methods of measuring viral transduction efficiency in vitro or in vivo are well known to one of ordinary skill in the art, including, for example, qPCR analysis, deep sequencing, western blotting, fluorometric analysis (such as fluorescent in situ hybridization (FISH), fluorescent reporter gene expression, immunofluorescence, FACS), etc.

In some embodiments provided herein are recombinant viruses, which may or may not be pseudotyped, that produce one or more therapeutic polypeptides for the treatment of an ATPase disorder or disease (e.g., Hailey-Hailey disease or Darier disease). In some embodiments, the one or more therapeutic polypeptides produced by the recombinant viruses described herein mediate or enhance a treatment of an ATPase disorder or disease (e.g., Hailey-Hailey disease or Darier disease). The present disclosure further provides therapeutic compositions comprising the recombinant viruses and methods of use in the treatment of an ATPase disorder or disease (e.g., Hailey-Hailey disease or Darier disease).

### V. Pharmaceutical Compositions and Formulations

Certain aspects of the present disclosure relate to pharmaceutical compositions or formulations comprising any of the recombinant nucleic acids (e.g., a recombinant herpes virus genome) and/or viruses (e.g., a herpes virus comprising a recombinant genome) described herein (such as a herpes simplex virus comprising a recombinant herpes simplex virus genome), and a pharmaceutically acceptable excipient or carrier.

In some embodiments, the pharmaceutical composition or formulation comprises any one or more of the viruses (e.g., herpes viruses) described herein. In some embodiments, the pharmaceutical composition or formulation comprises from about 10⁴ to about 10¹² plaque forming units (PFU)/mL of the virus. For example, the pharmaceutical composition or formulation may comprise from about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10⁴ to about 10¹¹, about 10⁵ to about 10¹¹, about 10⁶ to about 10¹¹, about 10⁷ to about 10¹¹, about 10⁸ to about 10¹¹, about 10⁹ to about 10¹¹, about 10¹⁰ to about 10¹¹, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁹ to about 10¹⁰, about 10⁴ to about 10⁹, about 10⁵ to about 10⁹, about 10⁶ to about 10⁹, about 10⁷ to about 10⁹, about 10⁸ to about 10⁹, about 10⁴ to about 10⁸, about 10⁵ to about 108, about 10⁶ to about 10⁸, about 10⁷ to about 10⁸, about 10⁴ to about 10⁷, about 10⁵ to about 10⁷, about 10⁶ to about 10⁷, about 10⁴ to about 10⁶, about 10⁵ to about 10⁶, or about 10⁴ to about 10⁵ PFU/mL of the virus. In some embodiments, the pharmaceutical composition or formulation comprises about 10⁴, about 10⁵, about 10⁶, about 10⁷, about 10⁸, about 10⁹, about 10¹⁰, about 10¹¹, or about 10¹² PFU/mL of the virus.

Pharmaceutical compositions and formulations can be prepared by mixing the active ingredient(s) (such as a recombinant nucleic acid and/or a virus) having the desired degree of purity with one or more pharmaceutically acceptable carriers or excipients. Pharmaceutically acceptable carriers or excipients are generally nontoxic to recipients at the dosages and concentrations employed, and may include, but are not limited to: buffers (such as phosphate, citrate, acetate, and other organic acids); antioxidants (such as ascorbic acid and methionine); preservatives (such as octadecyldimethylbenzyl ammonium chloride, benzalkonium chloride, benzethonium chloride, phenol, butyl or benzyl alcohol, alkyl parabens, catechol, resorcinol, cyclohexanol, 3-pentanol, and m-cresol); amino acids (such as glycine, glutamine, asparagine, histidine, arginine, or lysine); low molecular weight (less than about 10 residues) polypeptides; proteins (such as serum albumin, gelatin, or immunoglobulins); polyols (such as glycerol, e.g., formulations including 1%, 2%, 3%, 4%, 5%, 6%, 7%, 8%, 9%, 10%, 11%, 12%, 13%, 14%, 15%, etc. glycerol); hydrophilic polymers (such as polyvinylpyrrolidone); monosaccharides, disaccharides, and other carbohydrates (including glucose, mannose, or dextrins); chelating agents (such as EDTA); sugars (such as sucrose, mannitol, trehalose, or sorbitol); salt-forming counter-ions (such as sodium); metal complexes (such as Zn-protein complexes); and/or non-ionic surfactants (such as polyethylene glycol (PEG)). A thorough discussion of pharmaceutically acceptable carriers is available in REMINGTON'S PHARMACEUTICAL SCIENCES (Mack Pub. Co., N. J. 1991).

In some embodiments, the pharmaceutical composition or formulation comprises one or more lipid (e.g., cationic lipid) carriers. In some embodiments, the pharmaceutical composition or formulation comprises one or more nanoparticle carriers. Nanoparticles are submicron (less than about 10⁰⁰ nm) sized drug delivery vehicles that can carry encapsulated drugs (such as synthetic small molecules, proteins, peptides, cells, viruses, and nucleic acid-based biotherapeutics) for rapid or controlled release. A variety of molecules (e.g., proteins, peptides, recombinant nucleic acids, etc.) can be efficiently encapsulated in nanoparticles using processes well known in the art. In some embodiments, a molecule “encapsulated” in a nanoparticle may refer to a molecule (such as a virus) that is contained within the nanoparticle or attached to and/or associated with the surface of the nanoparticle, or any combination thereof. Nanoparticles for use in the compositions or formulations described herein may be any type of biocompatible nanoparticle known in the art, including, for example, nanoparticles comprising poly (lactic acid), poly(glycolic acid), PLGA, PLA, PGA, and any combinations thereof (see e.g., Vauthier et al. Adv Drug Del Rev. (2003) 55:519-48; US2007/0148074; US2007/0092575; US2006/0246139; U.S. Pat. Nos. 5,753,234; 7,081,483; and WO2006/052285).

In some embodiments, the pharmaceutically acceptable carrier or excipient may be adapted for or suitable for any administration route known in the art, including, for example, intravenous, intramuscular, subcutaneous, cutaneous, oral, nasal, intratracheal, sublingual, buccal, topical, transdermal, intradermal, intraperitoneal, intraorbital, subretinal, intravitreal, transmucosal, intraarticular, by implantation, by inhalation, intrathecal, intraventricular, and/or intranasal administration. In some embodiments, the pharmaceutical composition or formulation is adapted for or suitable for any administration route known in the art, including, for example, intravenous, intramuscular, subcutaneous, cutaneous, oral, nasal, intratracheal, sublingual, buccal, topical, transdermal, intradermal, intraperitoneal, intraorbital, intravitreal, subretinal, transmucosal, intraarticular, by implantation, by inhalation, intrathecal, intraventricular, and/or intranasal administration. In some embodiments, the pharmaceutically acceptable carrier or excipient is adapted for or suitable for topical, transdermal, subcutaneous, intradermal, and/or transmucosal administration. In some embodiments, the pharmaceutical composition or formulation is adapted for or suitable for topical, transdermal, subcutaneous, intradermal, and/or transmucosal administration. In some embodiments, the pharmaceutically acceptable carrier or excipient is adapted for or suitable for topical, transdermal, subcutaneous, and/or intradermal administration. In some embodiments, the pharmaceutical composition or formulation is adapted for or suitable for topical, transdermal, subcutaneous, and/or intradermal administration. In some embodiments, the pharmaceutically acceptable carrier or excipient is adapted for or suitable for topical, transdermal, and/or intradermal administration. In some embodiments, the pharmaceutical composition or formulation is adapted for or suitable for topical, transdermal, and/or intradermal administration. In some embodiments, the pharmaceutically acceptable carrier or excipient is adapted for or suitable for topical administration. In some embodiments, the pharmaceutical composition or formulation is adapted for or suitable for topical administration. In some embodiments, the pharmaceutically acceptable carrier or excipient is adapted for or suitable for oral, sublingual, nasal, or buccal administration, or administration via inhalation. In some embodiments, the pharmaceutical composition or formulation is adapted for or suitable for oral, sublingual, nasal, or buccal administration, or administration via inhalation. In some embodiments, the pharmaceutically acceptable carrier or excipient is adapted for or suitable for topical (to the eye), intravitreal, subretinal or intraorbital administration. In some embodiments, the pharmaceutical composition or formulation is adapted for or suitable for topical (to the eye), intravitreal, subretinal, or intraorbital administration.

Examples of carriers or excipients adapted for or suitable for use in pharmaceutical compositions or formulations of the present disclosure may include, but are not limited to, ointments, oils, pastes, creams, aerosols, suspensions, emulsions, fatty ointments, gels (e.g., methylcellulose gels, such as carboxy methylcellulose, hydroxypropyl methylcellulose, etc.), powders, liquids, lotions, solutions, sprays, patches (e.g., transdermal patches or microneedle patches), adhesive strips, a microneedle or microneedle arrays, and inhalants. In some embodiments, the carrier or excipient (e.g., the pharmaceutically acceptable carrier or excipient) comprises one or more (e.g., one or more, two or more, three or more, four or more, five or more, etc.) of an ointment, oil, paste, cream, aerosol, suspension, emulsion, fatty ointment, gel, powder, liquid, lotion, solution, spray, patch, adhesive strip, and an inhalant. In some embodiments, the carrier comprises a patch (e.g. a patch that adheres to the skin), such as a transdermal patch or microneedle patch. In some embodiments, the carrier comprises a microneedle or microneedle array. Methods for making and using microneedle arrays suitable for composition delivery are generally known in the art (see e.g., Kim Y. et al. “Microneedles for drug and vaccine delivery”. Advanced Drug Delivery Reviews 2012, 64 (14): 1547-68).

In some embodiments, the pharmaceutically acceptable carrier or excipient may be adapted for or suitable for any administration route known in the art, including, for example, intravenous, intramuscular, subcutaneous, cutaneous, oral, intranasal, intratracheal, sublingual, buccal, topical, transdermal, intradermal, intraperitoneal, intraorbital, intravitreal, subretinal, transmucosal, intraarticular, by implantation, by inhalation, intrathecal, intraventricular, and/or intranasal administration. In some embodiments, the pharmaceutically acceptable carrier or excipient may be adapted for or suitable for topical administration.

In some embodiments, the pharmaceutical composition or formulation is adapted for or suitable for any administration route known in the art, including, for example, intravenous, intramuscular, subcutaneous, cutaneous, oral, intranasal, intratracheal, sublingual, buccal, topical, transdermal, intradermal, intraperitoneal, intraorbital, intravitreal, subretinal, transmucosal, intraarticular, by implantation, by inhalation, intrathecal, intraventricular, or intranasal administration. In some embodiments, the pharmaceutical composition or formulation is adapted for or suitable for topical administration.

In some embodiments, the pharmaceutically acceptable carrier or excipient may be adapted for or suitable for any administration route known in the art, including, for example, intravenous, intramuscular, subcutaneous, cutaneous, oral, intranasal, intratracheal, sublingual, buccal, topical, transdermal, intradermal, intraperitoneal, intraorbital, intravitreal, subretinal, suprachoroidal, transmucosal, intraarticular, by implantation, by inhalation, intrathecal, intraventricular, and/or intranasal administration.

In some embodiments, the pharmaceutical composition or formulation is adapted for or suitable for any administration route known in the art, including, for example, intravenous, intramuscular, subcutaneous, cutaneous, oral, intranasal, intratracheal, sublingual, buccal, topical, transdermal, intradermal, intraperitoneal, intraorbital, intravitreal, subretinal, suprachoroidal, transmucosal, intraarticular, by implantation, by inhalation, intrathecal, intraventricular, or intranasal administration.

In some embodiments, the pharmaceutical composition or formulation further comprises one or more additional components. Examples of additional components may include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); wetting agents (e.g., sodium lauryl sulphate, etc.); salt solutions; alcohols; polyethylene glycols; gelatin; lactose; amylase; magnesium stearate; talc; silicic acid; viscous paraffin; hydroxymethylcellulose; polyvinylpyrrolidone; sweetenings; flavorings; perfuming agents; colorants; moisturizers; sunscreens; antibacterial agents; agents able to stabilize polynucleotides or prevent their degradation, and the like. In some embodiments, the pharmaceutical composition or formulation comprises a methylcellulose gel (e.g., hydroxypropyl methylcellulose, carboxy methylcellulose, etc.). In some embodiments, the pharmaceutical composition or formulation comprises a phosphate buffer. In some embodiments, the pharmaceutical composition or formulation comprises glycerol (e.g., at about 1%, about 2%, about 3%, about 4%, about 5%, about 6%, about 7%, about 8%, about 9%, about 10%, about 11%, about 12%, about 13%, about 14%, about 15%, etc.). In some embodiments, the pharmaceutical composition or formulation comprises a phosphate buffer and glycerol.

Pharmaceutical compositions and formulations to be used for in vivo administration are generally sterile. Sterility may be readily accomplished, e.g., by filtration through sterile filtration membranes.

In some embodiments, any of the recombinant nucleic acids, viruses, and/or pharmaceutical compositions or formulations described herein may be used to deliver one or more polynucleotides encoding a Calcium-transporting ATPase type 2C member 1 polypeptide into one or more cells of a subject (e.g., one or more ATP2C1-deficient cells, one or more cells harboring an ATP2C1 gene mutation, etc.). In some embodiments, any of the recombinant nucleic acids, viruses, and/or pharmaceutical compositions or formulations may be used in a therapy. In some embodiments, any of the recombinant nucleic acids, viruses, and/or pharmaceutical compositions or formulations described herein may be used in the treatment of a disease or condition that would benefit from the expression of a Calcium-transporting ATPase type 2C member 1 polypeptide (e.g., a disease/disorder/defect associated with an ATP2C1 deficiency and/or a disease associated with a ATP2C1gene mutation (such as Haily-Hailey disease)). In some embodiments, any of the recombinant nucleic acids, viruses, and/or pharmaceutical compositions or formulations described herein may be used in the treatment of one or more signs or symptoms of Hailey-Hailey disease. Signs and symptoms of Hailey-Hailey disease include, but are not limited to: erythema, papulovesicles, skin fissures, blistering, wounding, and/or scarring of the skin; granulation tissue; skin erosion; deformity of the fingernails and/or toenails; tightening and/or thinning of the skin; contractures; blistering and/or scarring of the mucosa; increased susceptibility to infection; dehydration; fluid loss; electrolyte imbalance; and any combinations thereof. In some embodiments, any of the recombinant nucleic acids, viruses, and/or pharmaceutical compositions or formulations described herein may be used for providing prophylactic, palliative, or therapeutic relief to one or more signs or symptoms of Hailey-Hailey disease.

In some embodiments, any of the recombinant nucleic acids, viruses, and/or pharmaceutical compositions or formulations described herein may be used in the preparation or manufacture of a medicament. In some embodiments, any of the recombinant nucleic acids, viruses, and/or pharmaceutical compositions or formulations described herein may be used in the preparation or manufacture of a medicament useful for delivering one or more polynucleotides encoding a Calcium-transporting ATPase type 2C member 1 polypeptide into one or more cells of a subject (e.g., one or more ATP2C1-deficient cells, one or more cells harboring an ATP2C1gene mutation, etc.). In some embodiments, any of the recombinant nucleic acids, viruses, and/or pharmaceutical compositions or formulations described herein may be used in the preparation or manufacture of a medicament useful for the treatment of a disease or condition that would benefit from the expression of a Calcium-transporting ATPase type 2C member 1polypeptide (e.g., a disease/disorder/defect associated with a laminin deficiency and/or a disease associated with an ATP2C1gene mutation (such as Hailey-Hailey disease)). In some embodiments, any of the recombinant nucleic acids, viruses, and/or pharmaceutical compositions or formulations described herein may be used in the preparation or manufacture of a medicament useful for the treatment of one or more signs or symptoms of a Hailey-Hailey disease. Signs and symptoms of Hailey-Hailey disease include, but are not limited to: erythema, papulovesicles, skin fissures, blistering, wounding, and/or scarring of the skin; granulation tissue; skin erosion; deformity of the fingernails and/or toenails; tightening and/or thinning of the skin; contractures; blistering and/or scarring of the mucosa; increased susceptibility to infection; dehydration; fluid loss; electrolyte imbalance; and any combinations thereof. In some embodiments, any of the recombinant nucleic acids, viruses, and/or pharmaceutical compositions or formulations described herein may be used in the preparation or manufacture of a medicament useful for the treatment of Hailey-Hailey disease.

In some embodiments, any of the recombinant nucleic acids, viruses, and/or pharmaceutical compositions or formulations described herein may be used to deliver one or more polynucleotides encoding a Sarcoplasmic/endoplasmic reticulum calcium ATPase 2 polypeptide into one or more cells of a subject (e.g., one or more ATP2A2-deficient cells, one or more cells harboring an ATP2A2 or ATP2A2 gene mutation, etc.). In some embodiments, any of the recombinant nucleic acids, viruses, and/or pharmaceutical compositions or formulations may be used in a therapy. In some embodiments, any of the recombinant nucleic acids, viruses, and/or pharmaceutical compositions or formulations described herein may be used in the treatment of a disease or condition that would benefit from the expression of a Sarcoplasmic/endoplasmic reticulum calcium ATPase 2polypeptide (e.g., a disease/disorder/defect associated with a ATP2A2 deficiency and/or a disease associated with an ATP2A2 or ATP2A2 gene mutation (such as Darier disease)). In some embodiments, any of the recombinant nucleic acids, viruses, and/or pharmaceutical compositions or formulations described herein may be used in the treatment of one or more signs or symptoms of Darier disease. Signs and symptoms of Darier disease deficiencies include, but are not limited to erythematous, papules, reddish-brown papules confluent papules, papillomatous plaques, plaques, plaques with a keratotic surface, and any combinations thereof. In some embodiments, any of the recombinant nucleic acids, viruses, and/or pharmaceutical compositions or formulations described herein may be used for providing prophylactic, palliative, or therapeutic relief to one or more signs or symptoms of Darier disease.

In some embodiments, any of the recombinant nucleic acids, viruses, and/or pharmaceutical compositions or formulations described herein may be used in the preparation or manufacture of a medicament. In some embodiments, any of the recombinant nucleic acids, viruses, and/or pharmaceutical compositions or formulations described herein may be used in the preparation or manufacture of a medicament useful for delivering one or more polynucleotides encoding a Sarcoplasmic/endoplasmic reticulum calcium ATPase 2 polypeptide into one or more cells of a subject (e.g., one or more ATP2A2-deficient cells, one or more cells harboring an ATP2A2 or ATP2A2 gene mutation, etc.). In some embodiments, any of the recombinant nucleic acids, viruses, and/or pharmaceutical compositions or formulations described herein may be used in the preparation or manufacture of a medicament useful for the treatment of a disease or condition that would benefit from the expression of a Sarcoplasmic/endoplasmic reticulum calcium ATPase 2 polypeptide (e.g., a disease/disorder/defect associated with an ATP2A2deficiency and/or a disease associated with an ATP2A2or ATP2A2gene mutation (such as Darier disease)). In some embodiments, any of the recombinant nucleic acids, viruses, and/or pharmaceutical compositions or formulations described herein may be used in the preparation or manufacture of a medicament useful for the treatment of one or more signs or symptoms of a Darier disease. Signs and symptoms of Darier disease include, but are not limited to: erythematous, papules, reddish-brown papules confluent papules, papillomatous plaques, plaques, plaques with a keratotic surface, and any combinations thereof. In some embodiments, any of the recombinant nucleic acids, viruses, and/or pharmaceutical compositions or formulations described herein may be used in the preparation or manufacture of a medicament useful for the treatment of Darier disease.

### VI. Methods

Certain aspects of the present disclosure relate to enhancing, increasing, augmenting, and/or supplementing the levels of one or more Calcium-transporting ATPase type 2C member 1 polypeptides (e.g., one or more human Calcium-transporting ATPase type 2C member 1 polypeptides) in one or more cells and/or in the extracellular matrix of a subject comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in an endogenous ATP2C1 gene. In some embodiments, the subject suffers from Hailey-Hailey disease.

In some embodiments, administration of the recombinant nucleic acid, virus, medicament, and/or pharmaceutical composition or formulation to the subject increases Calcium-transporting ATPase type 2C member 1 levels (transcript or protein levels) by at least about 25% in one or more contacted or treated cells of the subject, as compared to the endogenous levels of the Calcium-transporting ATPase type 2C member 1 in one or more corresponding untreated cells of the subject. In some embodiments, administration of the recombinant nucleic acid, virus, medicament, and/or pharmaceutical composition or formulation to the subject increases Calcium-transporting ATPase type 2C member 1 levels (transcript or protein levels) by at least about 25%, at least about 30%, at least about 40%, at least about 50%, at least about 50%, at least about 60%, at least about 70%, at least about 80%, at least about 90%, at least about 95%, at least about 99%, or more in one or more contacted or treated cells of the subject, as compared to the endogenous levels of the Calcium-transporting ATPase type 2C member 1 in one or more corresponding untreated cells of the subject. In some embodiments, administration of the recombinant nucleic acid, virus, medicament, and/or pharmaceutical composition or formulation to the subject increases Calcium-transporting ATPase type 2C member 1 levels (transcript or protein levels) by at least about 2-fold in one or more contacted or treated cells of the subject, as compared to the endogenous levels of the Calcium-transporting ATPase type 2C member 1 in one or more corresponding untreated cells of the subject. For example, administration of the recombinant nucleic acid, virus, medicament, and/or pharmaceutical composition or formulation may increase Calcium-transporting ATPase type 2C member 1 levels (transcript or protein levels) by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 10⁰-fold, at least about 250-fold, at least about 500-fold, at least about 750-fold, at least about 10⁰⁰-fold, or more in one or more contacted or treated cells of the subject, as compared to the endogenous levels of the Calcium-transporting ATPase type 2C member 1 in one or more corresponding untreated cells of the subject. In some embodiments, the one or more contacted or treated cells are one or more cells of the epidermis, dermis, and/or mucosa. Methods of measuring transcript or protein levels from a sample are well known to one of ordinary skill in the art, including, for example, by qPCR, western blot, mass spectrometry, etc.

Other aspects of the present disclosure relate to enhancing, increasing, augmenting, and/or supplementing cell adhesion of one or more cells of a subject comprising administering to the subject any of the recombinant nucleic acids, viruses, medicaments, and/or pharmaceutical compositions or formulations described herein. In some embodiments, the enhanced, increased, augmented, or supplemented cell adhesion is in comparison to the levels of cell adhesion of a corresponding cell that has not been contacted with or administered the recombinant nucleic acids, viruses, medicaments, and/or pharmaceutical compositions described herein. In some embodiments, the one or more cells are one or more epidermal, dermal, and/or mucosal cells. In some embodiments, the one or more cells are one or more cells of the skin of the subject. In some embodiments, the cell adhesion is integrin-mediated cell adhesion. Methods of measuring cell adhesion are known to one of ordinary skill in the art (see e.g., Boettiger D. Methods Enzymol. 2007; 426:1-25). In some embodiments, the subject is a human. In some embodiments, the subject's genome comprises a mutation (e.g., a loss-of-function mutation, a pathogenic variant) in an endogenous ATP2C1 gene. In some embodiments, the subject suffers from Hailey-Hailey disease.

Other aspects of the present disclosure relate to providing prophylactic, palliative, or therapeutic relief to one or more signs or symptoms of Hailey-Hailey disease in a subject in need thereof comprising administering to the subject any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in an endogenous ATP2C1 gene. In some embodiments, the subject has Hailey-Hailey disease or is at risk of developing Hailey-Hailey disease. Signs and/or symptoms of Hailey-Hailey disease may include, but are not limited to, erythema, papulovesicles, skin fissures, blistering, wounding, and/or scarring of the skin; granulation tissue; skin erosion; deformity of the fingernails and/or toenails; tightening and/or thinning of the skin; contractures; blistering and/or scarring of the mucosa; increased susceptibility to infection; dehydration; fluid loss; electrolyte imbalance; and any combinations thereof.

In some embodiments, the recombinant nucleic acid expresses the encoded Calcium-transporting ATPase type 2C member 1 protein when the recombinant nucleic acid is delivered into one or more target cells of a subject. In some embodiments, expression of the Calcium-transporting ATPase type 2C member 1 protein enhances, increases, augments, and/or supplements the levels of Calcium-transporting ATPase type 2C member 1 in one or more target cells. In some embodiments, expression of the Calcium-transporting ATPase type 2C member 1 protein enhances, increases, augments, and/or supplements the levels of Calcium-transporting ATPase type 2C member 1 secreted by one or more target cells. In some embodiments, expression of the Calcium-transporting ATPase type 2C member 1 protein enhances, increases, augments, and/or supplements the stability of the extracellular matrix in the subject. In some embodiments, expression of the Calcium-transporting ATPase type 2C member 1 protein treats an ATP2C1 deficiency in a Hailey-Hailey patient. In some embodiments, expression of the Calcium-transporting ATPase type 2C member 1 protein provides prophylactic, palliative, or therapeutic relief of one or more signs or symptoms in a Hailey-Hailey patient.

Certain aspects of the present disclosure relate to enhancing, increasing, augmenting, and/or supplementing the levels of one or more Sarcoplasmic/endoplasmic reticulum calcium ATPase 2 polypeptides (e.g., one or more human Sarcoplasmic/endoplasmic reticulum calcium ATPase 2 polypeptides) in one or more cells and/or in the extracellular matrix of a subject comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in an endogenous ATP2A2 gene. In some embodiments, the subject suffers from Darier disease.

In some embodiments, administration of the recombinant nucleic acid, virus, medicament, and/or pharmaceutical composition or formulation to the subject increases Sarcoplasmic/endoplasmic reticulum calcium ATPase 2 levels (transcript or protein levels) by at least about 25% in one or more contacted or treated cells of the subject, as compared to the endogenous levels of the Sarcoplasmic/endoplasmic reticulum calcium ATPase 2 in one or more corresponding untreated cells of the subject. In some embodiments, administration of the recombinant nucleic acid, virus, medicament, and/or pharmaceutical composition or formulation to the subject increases Sarcoplasmic/endoplasmic reticulum calcium ATPase 2 levels (transcript or protein levels) by at least about 25%, at least about 30%, at least about 40%, at least about 50%, at least about 50%, at least about 60%, at least about 70%, at least about 80%, at least about 90%, at least about 95%, at least about 99%, or more in one or more contacted or treated cells of the subject, as compared to the endogenous levels of the Sarcoplasmic/endoplasmic reticulum calcium ATPase 2 in one or more corresponding untreated cells of the subject. In some embodiments, administration of the recombinant nucleic acid, virus, medicament, and/or pharmaceutical composition or formulation to the subject increases Sarcoplasmic/endoplasmic reticulum calcium ATPase 2 levels (transcript or protein levels) by at least about 2-fold in one or more contacted or treated cells of the subject, as compared to the endogenous levels of the Sarcoplasmic/endoplasmic reticulum calcium ATPase 2 in one or more corresponding untreated cells of the subject. For example, administration of the recombinant nucleic acid, virus, medicament, and/or pharmaceutical composition or formulation may increase Sarcoplasmic/endoplasmic reticulum calcium ATPase 2 levels (transcript or protein levels) by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 10⁰-fold, at least about 250-fold, at least about 500-fold, at least about 750-fold, at least about 10⁰⁰-fold, or more in one or more contacted or treated cells of the subject, as compared to the endogenous levels of the Sarcoplasmic/endoplasmic reticulum calcium ATPase 2 in one or more corresponding untreated cells of the subject. In some embodiments, the one or more contacted or treated cells are one or more cells of the epidermis, dermis, and/or mucosa. Methods of measuring transcript or protein levels from a sample are well known to one of ordinary skill in the art, including, for example, by qPCR, western blot, mass spectrometry, etc.

Other aspects of the present disclosure relate to enhancing, increasing, augmenting, and/or supplementing cell adhesion of one or more cells of a subject comprising administering to the subject any of the recombinant nucleic acids, viruses, medicaments, and/or pharmaceutical compositions or formulations described herein. In some embodiments, the enhanced, increased, augmented, or supplemented cell adhesion is in comparison to the levels of cell adhesion of a corresponding cell that has not been contacted with or administered the recombinant nucleic acids, viruses, medicaments, and/or pharmaceutical compositions described herein. In some embodiments, the one or more cells are one or more epidermal, dermal, and/or mucosal cells. In some embodiments, the one or more cells are one or more cells of the skin of the subject. In some embodiments, the cell adhesion is integrin-mediated cell adhesion. Methods of measuring cell adhesion are known to one of ordinary skill in the art (see e.g., Boettiger D. Methods Enzymol. 2007; 426:1-25). In some embodiments, the subject is a human. In some embodiments, the subject's genome comprises a mutation (e.g., a loss-of-function mutation, a pathogenic variant) in an endogenous ATP242 gene. In some embodiments, the subject suffers from Darier disease.

Other aspects of the present disclosure relate to providing prophylactic, palliative, or therapeutic relief to one or more signs or symptoms of Darier disease in a subject in need thereof comprising administering to the subject any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in an endogenous ATP2A2 gene. In some embodiments, the subject has Darier disease or is at risk of developing Darier disease. Signs and/or symptoms of Darier disease may include, but are not limited to, erythematous, papules, reddish-brown papules confluent papules, papillomatous plaques, plaques, plaques with a keratotic surface, and any combinations thereof.

In some embodiments, the recombinant nucleic acid expresses the encoded Sarcoplasmic/endoplasmic reticulum calcium ATPase 2 protein when the recombinant nucleic acid is delivered into one or more target cells of a subject. In some embodiments, expression of the Sarcoplasmic/endoplasmic reticulum calcium ATPase 2 protein enhances, increases, augments, and/or supplements the levels of Sarcoplasmic/endoplasmic reticulum calcium ATPase 2 in one or more target cells. In some embodiments, expression of the Sarcoplasmic/endoplasmic reticulum calcium ATPase 2 protein enhances, increases, augments, and/or supplements the levels of Sarcoplasmic/endoplasmic reticulum calcium ATPase 2 secreted by one or more target cells. In some embodiments, expression of the Sarcoplasmic/endoplasmic reticulum calcium ATPase 2 protein enhances, increases, augments, and/or supplements the stability of the extracellular matrix in the subject. In some embodiments, expression of the Sarcoplasmic/endoplasmic reticulum calcium ATPase 2 protein treats an ATP2A2 deficiency in a Darier patient. In some embodiments, expression of the Sarcoplasmic/endoplasmic reticulum calcium ATPase 2 protein provides prophylactic, palliative, or therapeutic relief of one or more signs or symptoms in a Darier patient.

The recombinant nucleic acids, viruses, medicaments, and/or pharmaceutical compositions or formulations described herein may be administered by any suitable method or route known in the art, including, without limitation, by oral administration, sublingual administration, buccal administration, topical administration, rectal administration, via inhalation, transdermal administration, subcutaneous injection, intradermal injection, intravenous injection, intra-arterial injection, intramuscular injection, intracardiac injection, intraosseous injection, intraperitoneal injection, transmucosal administration, vaginal administration, intravitreal administration, intraorbital administration, subretinal administration, subconjunctival administration (e.g., the use of subconjunctival depots), suprachoroidal administration, intra-articular administration, peri-articular administration, local administration, epicutaneous administration, or any combinations thereof. The present disclosure thus encompasses methods of delivering any of the recombinant nucleic acids, viruses, medicaments, and/or pharmaceutical compositions or formulations described herein to an individual (or a specific site or tissue thereof).

In some embodiments, the recombinant nucleic acid, virus, medicaments, and/or pharmaceutical composition or formulation used in the methods of the present disclosure is administered cutaneously, topically, transdermally, subcutaneously, intradermally, transmucosally, sublingually, nasally, buccally, intravitreally, subretinally, subconjunctivally, suprachoroidally, intraarticularly, or via inhalation to the subject. In some embodiments, the recombinant nucleic acid, virus, medicament, and/or pharmaceutical composition or formulation is administered topically, transdermally, subcutaneously, intradermally or transmucosally to the subject. In some embodiments, the recombinant nucleic acid, virus, medicament, and/or pharmaceutical composition or formulation is administered topically, transdermally, or intradermally to the subject. In some embodiments, the recombinant nucleic acid, virus, medicament, and/or pharmaceutical composition or formulation is administered topically to the subject (e.g., to a site of skin disease of a subject). In some embodiments, the recombinant nucleic acid, virus, medicament, and/or pharmaceutical composition or formulation is administered intradermally to the subject. In some embodiments, the recombinant nucleic acid, virus, medicament, and/or pharmaceutical composition or formulation is administered orally, sublingually, buccally, nasally, or via inhalation to the subject. In some embodiments, the recombinant nucleic acid, virus, medicament, and/or pharmaceutical composition or formulation is administered orally or via inhalation to the subject. In some embodiments, the recombinant nucleic acid, virus, medicament, and/or pharmaceutical composition or formulation is administered intraorbitally, intravitreally, subretinally, subconjunctivally, suprachoroidally, or topically (to the eye) of the subject.

The recombinant nucleic acids, viruses, medicaments, and/or pharmaceutical compositions or formulations described herein may be administered by any suitable method or route known in the art, including, without limitation, via injection, via injection to the eye, via subretinal injection, via intraocular injection, via intravitreal injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, suprachoroidally, intranasally, intratracheally, sublingually, buccally, rectally, via inhalation, transdermally, subcutaneously, intradermally, intravenously, intraarterially, intramuscularly, intracardially, intraosseously, intraperitoneally, transmucosally, vaginally, intravitreally, intraorbitally, subretinally, intraarticularly, peri-articularly, locally, epicutaneously, or any combinations thereof. The present disclosure thus encompasses methods of delivering any of the recombinant nucleic acids, viruses, medicaments, or pharmaceutical compositions or formulations described herein to an individual (e.g., an individual having an eye condition or disease). In some embodiments, the recombinant nucleic acids, viruses, medicaments, and/or pharmaceutical compositions or formulations described herein are administered via injection, via injection to the eye, via subretinal injection, via intraocular injection, via intravitreal injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally.

In some embodiments, the recombinant nucleic acid, virus, medicament, and/or pharmaceutical composition or formulation is administered once to the subject. In some embodiments, the recombinant nucleic acid, virus, medicament, and/or pharmaceutical composition or formulation is administered at least twice (e.g., at least 2 times, at least 3 times, at least 4 times, at least 5 times, at least 10 times, etc.) to the subject. In some embodiments, at least about 1 hour (e.g., at least about 1 hour, at least about 6 hours, at least about 12 hours, at least about 18 hours, at least about 1 day, at least about 2 days, at least about 3 days, at least about 4 days, at least about 5 days, at least about 6 days, at least about 7 days, at least about 15 days, at least about 20 days, at least about 30 days, at least about 40 days, at least about 50 days, at least about 60 days, at least about 70 days, at least about 80 days, at least about 90 days, at least about 100 days, at least about 120 days, etc.) pass between administrations (e.g., between the first and second administrations, between the second and third administrations, etc.). In some embodiments, the recombinant nucleic acid, virus, medicament, and/or pharmaceutical composition or formulation is administered one, two, three, four, five or more times per day to the subject. In some embodiments, the recombinant nucleic acid, virus, medicament, and/or pharmaceutical composition or formulation is administered to one or more affected (e.g., one or more regions displaying one or more signs or symptoms of Hailey-Hailey disease or Darier disease) and/or unaffected areas of the subject.

In some embodiments, one or more portions of the skin of the subject is abraded or made more permeable prior to treatment with a recombinant nucleic acid, virus, medicament, and/or pharmaceutical composition or formulation described herein. Any suitable method of abrading the skin or increasing skin permeability known in the art may be used, including, for example, use of a dermal roller, repeated use of adhesive strips to remove layers of skin cells (tape stripping), scraping with a scalpel or blade, use of sandpaper, use of chemical permeation enhancers or electrical energy, use of sonic or ultrasonic energy, use of light (e.g., laser) energy, use of micron-sized needles or blades with a length suitable to pierce but not completely pass through the epidermis, etc.

### VII. Host Cells

Certain aspects of the present disclosure relate to one or more host cells comprising any of the recombinant nucleic acids described herein. Any suitable host cell (prokaryotic or eukaryotic) known in the art may be used, including, for example: prokaryotic cells including eubacteria, such as Gram-negative or Gram-positive organisms, for example Enterobacteriaceae such as Escherichia (e.g., E. coli), Enterobacter, Erminia, Klebsiella, Proteus, Salmonella (e.g., S. typhimurium), Serratia (e.g., S. marcescans), and Shigella, as well as Bacilli such as B. subtilis and B. licheniformis; fungal cells (e.g., S. cerevisiae); insect cells (e.g., S2 cells, etc.); and mammalian cells, including monkey kidney CV1 line transformed by SV40 (COS-7, ATCC CRL 1651), human embryonic kidney line (293 or 293 cells subcloned for growth in suspension culture), baby hamster kidney cells (BHK, ATCC CCL 10), mouse Sertoli cells (TM4), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TRI cells, MRC 5 cells, FS4 cells, human hepatoma line (Hep G2), Chinese hamster ovary (CHO) cells, including DHFR″ CHO cells, and myeloma cell lines such as NS0 and Sp2/0. In some embodiments, the host cell is a human or non-human primate cell. In some embodiments, the host cells are cells from a cell line. Examples of suitable host cells or cell lines may include, but are not limited to, 293, HeLa, SH-Sy5y, Hep G2, CACO-2, A549, L929, 3T3, K562, CHO-K1, MDCK, HUVEC, Vero, N20, COS-7, PSN1, VCaP, CHO cells, and the like.

In some embodiments, the recombinant nucleic acid is a herpes simplex viral vector. In some embodiments, the recombinant nucleic acid is a herpes simplex virus amplicon. In some embodiments, the recombinant nucleic acid is an HSV-1 amplicon or HSV-1 hybrid amplicon. In some embodiments, a host cell comprising a helper virus is contacted with an HSV-1 amplicon or HSV-1 hybrid amplicon described herein, resulting in the production of a virus comprising one or more recombinant nucleic acids described herein. In some embodiments, the virus is collected from the supernatant of the contacted host cell. Methods of generating virus by contacting host cells comprising a helper virus with an HSV-1 amplicon or HSV-1 hybrid amplicon are known in the art.

In some embodiments, the host cell is a complementing host cell. In some embodiments, the complementing host cell expresses one or more genes that are inactivated in any of the viral vectors described herein. In some embodiments, the complementing host cell is contacted with a recombinant herpes virus genome (e.g., a recombinant herpes simplex virus genome) described herein. In some embodiments, contacting a complementing host cell with a recombinant herpes virus genome results in the production of a herpes virus comprising one or more recombinant nucleic acids described herein. In some embodiments, the virus is collected from the supernatant of the contacted host cell. Methods of generating virus by contacting complementing host cells with a recombinant herpes simplex virus are generally described in WO2015/009952, WO2017/176336, WO2019/200163, and/or WO2019/210219.

### VIII. Articles of Manufacture or Kits

Certain aspects of the present disclosure relate to an article of manufacture or a kit comprising any of the recombinant nucleic acids, viruses, medicaments, and/or pharmaceutical compositions or formulations described herein. In some embodiments, the article of manufacture or kit comprises a package insert comprising instructions for administering the recombinant nucleic acid, virus, medicament, and/or pharmaceutical composition or formulation.

Suitable containers for the recombinant nucleic acids, viruses, medicaments, and/or pharmaceutical compositions or formulations may include, for example, bottles, vials, bags, tubes, and syringes. The container may be formed from a variety of materials such as glass, plastic (such as polyvinyl chloride or polyolefin), or metal alloy (such as stainless steel or hastelloy). In some embodiments, the container comprises a label on, or associated with the container, wherein the label indicates directions for use. The article of manufacture or kit may further include other materials desirable from a commercial and user standpoint, including other buffers, diluents, filters, inhalers, nebulizers, intranasal administration devices, a package insert, and the like.

### IX. Enumerated Embodiments

Embodiment 1: a recombinant herpes virus genome comprising one or more polynucleotides encoding an ATPase polypeptide.

Embodiment 2: the recombinant herpes virus genome of embodiment 1, wherein the recombinant herpes virus genome is replication competent.

Embodiment 3: the recombinant herpes virus genome of embodiment 1 or 2, wherein the recombinant herpes virus genome is replication defective.

Embodiment 4: the recombinant herpes virus genome of any of embodiments 1-3, wherein the recombinant herpes virus genome is selected from the group consisting of a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Epstein-Barr virus genome, a recombinant Kaposi's sarcoma-associated herpesvirus genome, and any derivatives thereof.

Embodiment 5: the recombinant herpes virus genome of any of embodiments 1-4, wherein the recombinant herpes virus genome is a recombinant herpes simplex virus genome.

Embodiment 6: the recombinant herpes virus genome of any of embodiments 1-5, wherein the recombinant herpes simplex virus genome is a recombinant herpes simplex virus type 1 (HSV-1) genome, a recombinant herpes simplex virus type 2 (HSV-2) genome, or any derivatives thereof.

Embodiment 7: the recombinant herpes virus genome of any of embodiments 1-6, wherein the recombinant herpes simplex virus genome is a recombinant herpes simplex virus type 1 (HSV-1) genome.

Embodiment 8: the recombinant herpes virus genome of any of embodiments 1-7, wherein the recombinant herpes simplex virus genome has been engineered to reduce or eliminate expression of one or more toxic herpes simplex virus genes.

Embodiment 9: the recombinant herpes virus genome of any of embodiments 1-8, wherein the recombinant herpes simplex virus genome comprises an inactivating mutation.

Embodiment 10: the recombinant herpes virus genome of any of embodiments 1-9, wherein the inactivating mutation is in a herpes simplex virus gene.

Embodiment 11: the recombinant herpes virus genome of any of embodiments 1-10, wherein the inactivating mutation is a deletion of at least a portion of the coding sequence of the herpes simplex virus gene.

Embodiment 12: the recombinant herpes virus genome of any of embodiments 1-11, wherein the herpes simplex virus gene is selected from the group consisting of Infected Cell Protein (ICP) 0, ICP4, ICP22, ICP27, ICP47, thymidine kinase (tk), Long Unique Region (UL) 41, and UL55.

Embodiment 13: the recombinant herpes virus genome of any of embodiments 1-12, wherein the recombinant herpes simplex virus genome comprises an inactivating mutation in one or both copies of the ICP4 gene.

Embodiment 14: the recombinant herpes virus genome of any of embodiments 1-13, wherein the recombinant herpes simplex virus genome comprises an inactivating mutation in the ICP22 gene.

Embodiment 15: the recombinant herpes virus genome of any of embodiments 1-14, wherein the recombinant herpes simplex virus genome comprises an inactivating mutation in the UL41 gene.

Embodiment 16: the recombinant herpes virus genome of any of embodiments 1-15, wherein the recombinant herpes simplex virus genome comprises an inactivating mutation in one or both copies of the ICPO gene.

Embodiment 17: the recombinant herpes virus genome of any of embodiments 1-16, wherein the recombinant herpes simplex virus genome comprises an inactivating mutation in the ICP27 gene.

Embodiment 18: the recombinant herpes virus genome of any of embodiments 1-17, wherein the recombinant herpes simplex virus genome comprises an inactivating mutation in the UL55 gene.

Embodiment 19: the recombinant herpes virus genome of any of embodiments 1-18, wherein the recombinant herpes simplex virus genome does not comprise an inactivating mutation in the ICP47 gene.

Embodiment 20: the recombinant herpes virus genome of any of embodiments 1-19, wherein the recombinant herpes simplex virus does not comprise an inactivating mutation in one or both copies of the ICP34.5 gene.

Embodiment 21: the recombinant herpes virus genome of any of embodiments 1-20, wherein the recombinant herpes simplex virus genome comprises the one or more polynucleotides encoding the ATPase polypeptide within one or both of the ICP4 viral gene loci.

Embodiment 22: the recombinant herpes virus genome of any of embodiments 1-21, wherein the recombinant herpes simplex virus genome comprises the one or more polynucleotides encoding the ATPase polypeptide within the ICP22 viral gene locus.

Embodiment 23: the recombinant herpes virus genome of any of embodiments 1-22, wherein the recombinant herpes simplex virus genome comprises the one or more polynucleotides encoding the ATPase polypeptide within the UL41 viral gene locus.

Embodiment 24: the recombinant herpes virus genome of any of embodiments 1-23, wherein the recombinant herpes simplex virus genome comprises the one or more polynucleotides encoding the ATPase polypeptide within one or both of the ICPO viral gene loci.

Embodiment 25: the recombinant herpes virus genome of any of embodiments 1-24, wherein the recombinant herpes simplex virus genome comprises the one or more polynucleotides encoding the ATPase polypeptide within the ICP27 viral gene locus.

Embodiment 26: the recombinant herpes virus genome of any of embodiments 1-25, wherein the recombinant herpes simplex virus genome comprises the one or more polynucleotides encoding the ATPase polypeptide within the UL55 viral gene locus.

Embodiment 27: the recombinant herpes virus genome of any of embodiments 1-26, wherein the ATPase polypeptide is a human polypeptide.

Embodiment 28: the recombinant herpes virus genome of any of embodiments 1-26, wherein the ATPase polypeptide is a calcium-transporting ATPase type 2C member 1 polypeptide or a sarcoplasmic/endoplasmic reticulum calcium ATPase 2 polypeptide.

Embodiment 29: the recombinant herpes virus genome of any of embodiments 1-27, wherein the ATPase polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 1 or SEQ ID NO: 2.

Embodiment 30: the recombinant herpes virus genome of any of embodiments 1-29, wherein the ATPase polypeptide is a calcium-transporting ATPase type 2C member 1 polypeptide.

Embodiment 31: the recombinant herpes virus genome of any of embodiments 1-30, wherein the calcium-transporting ATPase type 2C member 1 polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 1.

Embodiment 32: the recombinant herpes virus genome of any of embodiments 1-29, wherein the ATPase polypeptide is a sarcoplasmic/endoplasmic reticulum calcium ATPase 2 polypeptide.

Embodiment 33: the recombinant herpes virus genome of embodiment 32, wherein the ATPase polypeptide is a sarcoplasmic/endoplasmic reticulum calcium ATPase 2 polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 2.

Embodiment 34: the recombinant herpes virus genome of any of embodiments 1-33, wherein the recombinant herpes virus genome has reduced cytotoxicity when introduced into a target cell as compared to a corresponding wild-type herpes virus genome.

Embodiment 35: the recombinant herpes virus genome of any of embodiments 1-34, wherein the target cell is a human cell.

Embodiment 36: the recombinant herpes virus genome of any of embodiments 1-35, wherein the target cell is a cell of the epidermis and/or dermis.

Embodiment 37: the recombinant herpes virus genome of any of embodiments 1-36, wherein the target cell is a keratinocyte, a basal cell, a melanocyte, a squamous cell, a Langerhans cell, a Merkel cell, or a fibroblast.

Embodiment 38: a herpes virus comprising the recombinant herpes virus genome of any one of embodiment 1-37.

Embodiment 39: the herpes virus of embodiment 38, wherein the herpes virus is replication competent.

Embodiment 40: the herpes virus of embodiment 38 or 39, wherein the herpes virus is replication defective.

Embodiment 41: the herpes virus of any one of embodiments 38-40, wherein the herpes virus has reduced cytotoxicity as compared to a corresponding wild-type herpes virus.

Embodiment 42: the herpes virus of any one of embodiments 38-41, wherein the herpes virus is selected from the group consisting of a herpes simplex virus, a varicella zoster virus, a human cytomegalovirus, a herpesvirus 6A, a herpesvirus 6B, a herpesvirus 7, an Epstein-Barr virus, and a Kaposi's sarcoma-associated herpesvirus.

Embodiment 43: the herpes virus of any one of embodiments 38-42, wherein the herpes virus is a herpes simplex virus.

Embodiment 44: the herpes virus of any one of embodiments 38-43, wherein the herpes simplex virus is a herpes simplex virus type 1 (HSV-1), a herpes simplex virus type 2 (HSV-2), or any derivatives thereof.

Embodiment 45: the herpes virus of any one of embodiments 38-44, wherein the herpes simplex virus is a herpes simplex virus type 1 (HSV-1).

Embodiment 46: a pharmaceutical composition comprising the recombinant herpes virus genome of any one of embodiments 1-37 or the herpes virus of any one of embodiments 38-48 and a pharmaceutically acceptable excipient.

Embodiment 47: the pharmaceutical composition of embodiment 46, wherein the pharmaceutical composition is suitable for topical, transdermal, subcutaneous, intradermal, oral, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, injection, or epicutaneous administration.

Embodiment 48: the pharmaceutical composition of embodiment 46 or 47, wherein the pharmaceutical composition is suitable for topical, transdermal, subcutaneous, intradermal, or transmucosal administration.

Embodiment 49: the pharmaceutical composition of any one of embodiments 46-48, wherein the pharmaceutical composition is suitable for topical, transdermal, or intradermal administration.

Embodiment 50: the pharmaceutical composition of any one of embodiments 46-49, wherein the pharmaceutical composition is suitable for topical administration.

Embodiment 51: the pharmaceutical composition of any one of embodiments 46-50, wherein the pharmaceutical composition is suitable for intradermal administration.

Embodiment 52: the pharmaceutical composition of any one of embodiments 46-51, wherein the pharmaceutical composition comprises a methylcellulose gel.

Embodiment 53: the pharmaceutical composition of any one of embodiments 46-52, wherein the pharmaceutical composition comprises a phosphate buffer.

Embodiment 54: the pharmaceutical composition of any one of embodiments 46-53, wherein the pharmaceutical composition comprises glycerol.

Embodiment 55: the pharmaceutical composition of any one of embodiments 46-54, wherein the pharmaceutical composition comprises a lipid carrier.

Embodiment 56: the pharmaceutical composition of any one of embodiments 46-55, wherein the pharmaceutical composition comprises a nanoparticle carrier.

Embodiment 57: the herpes virus of any one of embodiments 38-45 or the pharmaceutical composition of any one of embodiments 46-56 for use as a medicament.

Embodiment 58: the herpes virus of any one of embodiments 38-45 or the pharmaceutical composition of any one of embodiments 46-56, for use in a therapy.

Embodiment 59: the herpes virus of any one of embodiments 38-45 or the pharmaceutical composition of any one of embodiments 46-56, for use in the treatment of an ATPase disorder or disease.

Embodiment 60: the use of embodiment 59, wherein the ATPase disorder or disease is autosomal dominant.

Embodiment 61: the use of embodiment 59 or 60, wherein the ATPase disorder or disease is a haploinsufficiency.

Embodiment 62: the use of any one of embodiments 59-61, wherein the ATPase disorder or disease is a haploinsufficiency in one or more cells of the epidermis and/or dermis.

Embodiment 63: the use of any one of embodiments 59-62, wherein the ATPase disorder or disease is an autosomal dominant genodermatosis.

Embodiment 64: the use of any one of embodiments 59-63, wherein the ATPase disorder or disease is Hailey-Hailey disease or Darier disease.

Embodiment 65: the use of any one of embodiments 59-64, wherein the ATPase disorder or disease is Hailey-Hailey disease.

Embodiment 66: the use of any one of embodiments 59-64, wherein the ATPase disorder or disease is Darier disease.

Embodiment 67: use of the herpes virus of any one of embodiments 38-45 or the pharmaceutical composition of any one of embodiments 46-56 in the manufacture of a medicament for an ATPase disorder or disease.

Embodiment 68: the use of embodiment 67, wherein the ATPase disorder or disease is

Hailey-Hailey disease or Darier disease.

Embodiment 69: the use of embodiment 67 or 68, wherein the ATPase disorder or disease is Hailey-Hailey disease.

Embodiment 70: the use of embodiment 67 or 68, wherein the ATPase disorder or disease is Darier disease.

Embodiment 71: a method of expressing, enhancing, increasing, augmenting, and/or supplementing the levels of an ATPase polypeptide in one or more cells of a subject, the method comprising administering to the subject an effective amount of the herpes virus of any one of embodiments 38-45 or the pharmaceutical composition of any one of embodiments 46-56.

Embodiment 72: the method of embodiment 71, wherein the one or more cells are one or more keratinocytes, basal cells, melanocytes, squamous cells, Langerhans cells, Merkel cells, or fibroblasts.

Embodiment 73: a method of providing prophylactic, palliative, or therapeutic relief of one or more signs or symptoms of an ATPase disorder or disease in a subject in need thereof, the method comprising administering to the subject an effective amount of the herpes virus of any one of embodiments 38-45 or the pharmaceutical composition of any one of embodiments 46-56.

Embodiment 74: a method of treating an ATPase disorder or disease in a subject in need thereof, the method comprising administering to the subject an effective amount of the herpes virus of any one of embodiments 38-45 or the pharmaceutical composition of any one of embodiments 46-56.

Embodiment 75: the method of embodiment 73 or 74, wherein the ATPase disorder or disease is Hailey-Hailey disease or Darier disease.

Embodiment 76: the method of any one of embodiments 73-75, wherein the ATPase disorder or disease is Hailey-Hailey disease.

Embodiment 77: the method of any one of embodiments 73-75, wherein the ATPase disorder or disease is Darier disease.

Embodiment 78: the method of any one of embodiments 71-77, wherein the subject is a human.

Embodiment 79: the method of any one of embodiments 71-78, wherein the subject's genome comprises a loss-of-function mutation in an ATP2C1 gene or an ATP2A2 gene.

Embodiment 80: the method of any one of embodiments 71-79, wherein the subject's genome comprises a loss-of-function mutation in an ATP2C1 gene.

Embodiment 81: the method of any one of embodiments 71-79, wherein the subject's genome comprises a loss-of-function mutation in an ATP2A2 gene.

Embodiment 82: the method of any one of embodiments 71-81, wherein the herpes virus or pharmaceutical composition is administered via injection, topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, intraarticularly, periarticularly, locally, or via inhalation to the subject.

Embodiment 83: the method of any one of embodiments 71-82, wherein the herpes virus or pharmaceutical composition is administered topically, transdermally, subcutaneously, intradermally, orally, transmucosally, or via inhalation to the subject.

Embodiment 84: the method of any one of embodiments 71-83, wherein the herpes virus of pharmaceutical composition is administered topically to the subject.

Embodiment 85: the method of any one of embodiments 71-83, wherein the herpes virus or pharmaceutical composition is administered intradermally to the subject.

Embodiment 86: the method of any one of embodiments 71-85, wherein the skin of the subject is abraded prior to administration.

The specification is considered to be sufficient to enable one skilled in the art to practice the present disclosure. Various modifications of the present disclosure in addition to those shown and described herein will become apparent to those skilled in the art from the foregoing description and fall within the scope of the appended claims.

### Examples

The present disclosure will be more fully understood by reference to the following examples. It should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Modified Herpes Simplex Virus Vectors Encoding an ATPase Polypeptide

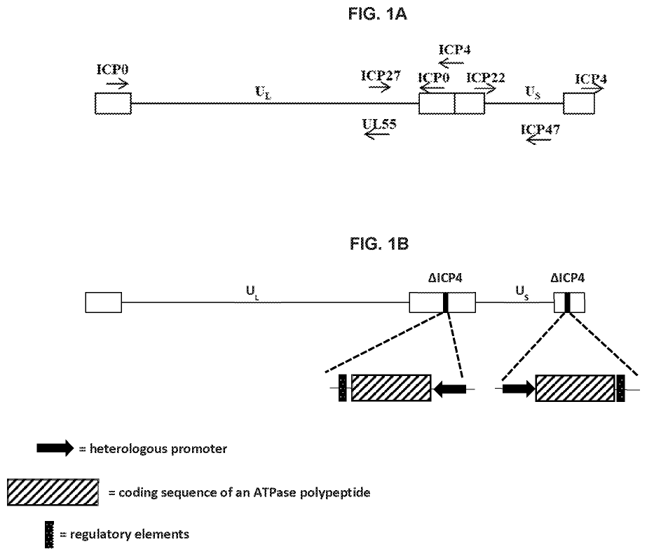

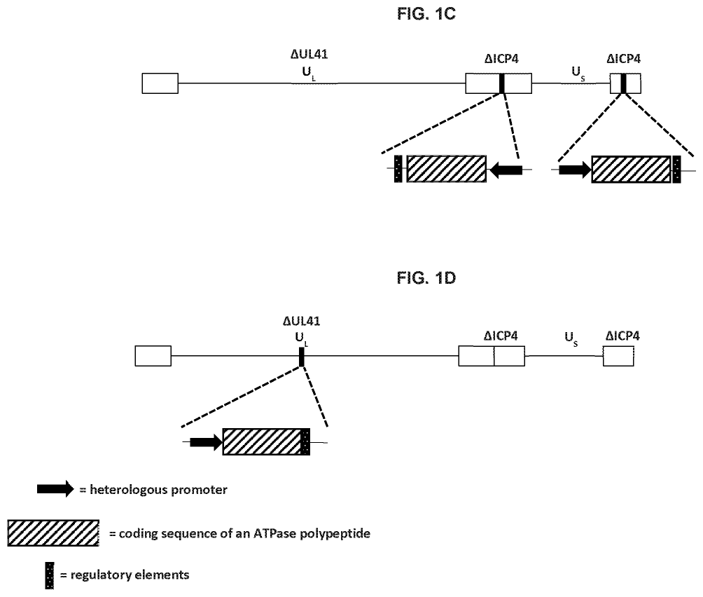

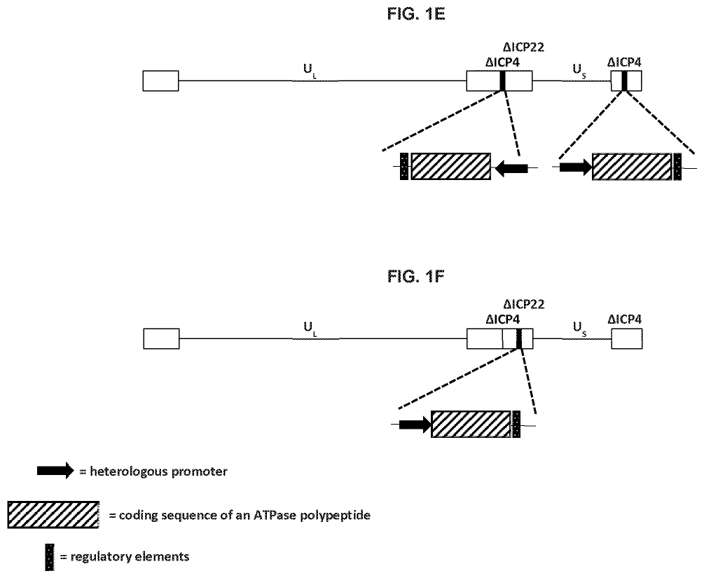

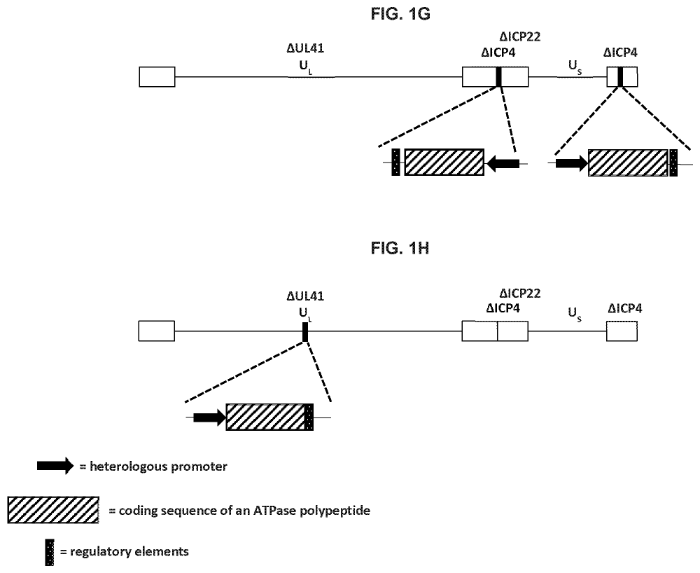

To make modified herpes simplex virus genome vectors capable of expressing ATPase polypeptides in a target mammalian cell (such as cells of the skin), a herpes simplex virus genome (FIG. 1A) is first modified to inactivate one or more herpes simplex virus genes. Such modifications may decrease the toxicity of the genome in mammalian cells. Next, variants of these modified/attenuated recombinant viral constructs are generated such that they carry one or more polynucleotides encoding the desired ATPase polypeptide. These variants include: a recombinant ΔICP4-modified HSV-1 genome comprising expression cassettes containing the coding sequence of an ATPase polypeptide (e.g., SEQ ID NO: 1 or SEQ ID NO: 2) under the control of a heterologous promoter integrated at each ICP4 locus (FIG. 1B); a recombinant ΔICP4/AUL41-modified HSV-1 genome comprising expression cassettes containing the coding sequence of an ATPase polypeptide (e.g., SEQ ID NO: 1 or SEQ ID NO: 2) under the control of a heterologous promoter integrated at each ICP4 locus (FIG. 1C); a recombinant ΔICP4/AUL41-modified HSV-1 genome comprising an expression cassette containing the coding sequence of an ATPase polypeptide (e.g., SEQ ID NO: 1 or SEQ ID NO: 2) under the control of a heterologous promoter integrated at the UL41 locus (FIG. 1D); a recombinant ΔICP4/ΔICP22-modified HSV-1 genome comprising expression cassettes containing the coding sequence of an ATPase polypeptide (e.g., SEQ ID NO: 1 or SEQ ID NO: 2) under the control of a heterologous promoter integrated at each ICP4 locus (FIG. 1E); a recombinant ΔICP4/ΔICP22-modified HSV-1 genome comprising an expression cassette containing the coding sequence of an ATPase polypeptide (e.g., SEQ ID NO: 1 or SEQ ID NO: 2) under the control of a heterologous promoter integrated at the ICP22 locus (FIG. 1F); a recombinant ΔICP4/AUL41/ΔICP22-modified HSV-1 genome comprising expression cassettes containing the coding sequence of an ATPase polypeptide (e.g., SEQ ID NO: 1 or SEQ ID NO: 2) under the control of a heterologous promoter integrated at each ICP4 locus (FIG. 1G); a recombinant ΔICP4/AUL41/ΔICP22-modified HSV-1 genome comprising an expression cassette containing the coding sequence of an ATPase polypeptide (e.g., SEQ ID NO: 1 or SEQ ID NO: 2) under the control of a heterologous promoter integrated at the UL41 locus (FIG. 1H); and a recombinant ΔICP4/AUL41/ΔICP22-modified HSV-1 genome comprising an expression cassette containing the coding sequence of an ATPase polypeptide (e.g., SEQ ID NO: 1 or SEQ ID NO: 2) under the control of a heterologous promoter integrated at the ICP22 locus (FIG. 1I).

These modified herpes simplex virus genome vectors are transfected into engineered cells that are modified to express one or more herpes simplex virus genes. These engineered cells secrete into the supernatant of the cell culture a replication-defective herpes simplex virus with the modified genomes packaged therein. The supernatant is then collected, concentrated, and sterile filtered.

Modified herpes simplex virus genome vectors described herein can express any one or more of the exemplary ATPase polypeptides in Table 1 below, in any suitable combination.

TABLE 1
Representative ATPase Polypeptides

| | | UniProt | | | NCBI |
|---|---|---|---|---|---|
| Amino acid | | Accession | Nucleic acid | Gene | Gene ID |
| SEQ ID NO. | Protein Name | No. | SEQ ID NO. | Name | No. |
| 1 | Calcium-transporting ATPase | P98194 | 3 | ATP2C1 | 27032 |
| | type 2C member 1 | | | | |
| 2 | Sarcoplasmic/endoplasmic | P16615 | 4 | ATP2A2 | 488 |
| | reticulum calcium ATPase 2 | | | | |

### Example 2: In Vitro and In Vivo Analysis of a Modified Herpes Simplex Virus Encoding Human Calcium-Transporting ATPase Type 2C Member 1 (ATP2C1) Polypeptide

Hailey-Hailey Disease is a genodermatosis with an estimated worldwide prevalence of 1 in 50,000 people and is characterized by blistering, painful sores, and pruritus. Hailey-Hailey Disease is caused by mutations in the ATP2C1 gene encoding the Calcium-transporting ATPase type 2C member 1 (ATP2C1) protein. The function of the Calcium-transporting ATPase type 2C member 1 is to pump cytosolic calcium into the Golgi apparatus, regulating the homeostasis of intracellular calcium. Calcium levels are involved in key signaling pathways that regulate cell function, survival, and growth, and act as a key regulator of differentiation and proliferation in epidermal keratinocytes, thus making these cells highly sensitive to changes in the levels of functional Calcium-transporting ATPase type 2C member 1 protein. While the exact mechanism of acantholysis in Hailey-Hailey Disease remains unknown, it is evident that ATP2C1 mutations lead to an imbalance of cytosolic calcium, disrupting the ability of epidermal keratinocytes to form strong intercellular connections and weakening the structural integrity of the skin. The impaired adhesion between cells likely contributes to the formation of blisters and erosions on the skin in Hailey-Hailey Disease patients.

The disease mainly manifests in areas of the body prone to excess moisture and heat, such as the armpits, groin, and neck, and affected skin can become thickened, scarred, and discolored over time. Hailey-Hailey Disease often presents during the second through fourth decades of life, following a chronic, relapsing course with periods of flare-ups and remissions often triggered by external factors like heat, sweating, friction, stress, or infection. The symptoms can range from mild to severe, with some individuals experiencing frequent and painful episodes. The skin lesions are not only physically troublesome but can also lead to significant psychological and emotional distress due to their chronic nature and visible appearance.

Currently, there is no cure for Hailey-Hailey Disease. Treatment focuses primarily on symptomatic relief and management of flare-ups, including use of topical corticosteroids, topical and oral antibiotics, retinoids, low doses of the opioid receptor antagonists naloxone and naltrexone, and lifestyle modifications to manage triggers such as avoiding excessive heat, friction, sweating, and wearing loose-fitting clothing to reduce irritation. While these treatments can help alleviate symptoms, they do not address the underlying molecular cause of the disease (e.g., lack of sufficient levels of functional Calcium-transporting ATPase type 2C member 1 protein). The use of an HSV-1 based vector platform technology provides an innovative treatment strategy for Hailey-Hailey Disease by providing functional full-length Calcium-transporting ATPase type 2C member 1 protein directly to the affected areas of the skin. The use of an HSV-1-based vector both capable of packaging full-length human ATP2C1 and possessing a natural tropism to human skin would therefore represent an innovative strategy for the treatment of Hailey-Hailey Disease, where topical application would lead to robust expression of Calcium-transporting ATPase type 2C member 1 protein in epidermal keratinocytes, thereby molecularly correcting the underlying disease state.

This study coupled the beneficial properties inherent to HSV-1, including its episomal lifecycle, high payload capacity, transduction efficiency, tropism for myriad human cell types (including those of the skin), and natural immune evasiveness, with a genetic modification strategy of targeted immediate early (IE) gene deletions to render a vector platform that is both replication-defective and non-cytotoxic. Accordingly, described herein is the use of a replication-defective, non-integrating HSV-1 based vector that has been engineered to deliver functional, full-length Calcium-transporting ATPase type 2C member 1 (termed HSV-ATP2C1) directly to the skin via topical administration to provide an effective redosable therapy for Hailey-Hailey Disease. In vitro

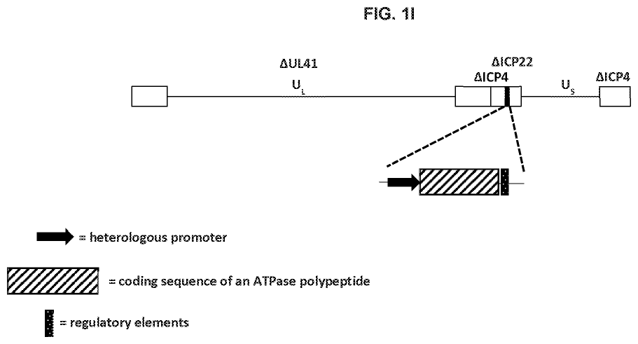

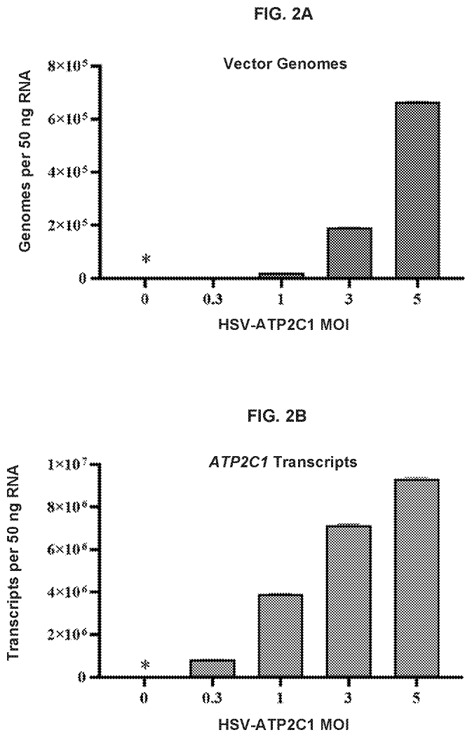

The HSV-ATP2C1 vector was first tested for its ability to transduce and deliver its transgene to human cells. HaCaT cells, an immortalized human keratinocyte line that is widely used in skin research and can recapitulate calcium-dependent primary epidermal keratinocyte proliferation and differentiation, were used as a clinically relevant model to verify both HSV-ATP2C1 transduction efficiency and ATP2C1 expression. Towards this end, cells were transduced with HSV-ATP2C1 at multiplicities of infection (MOIs) of 0.3, 1, 3, and 5. Mock-transduced cells (MOI 0) were used as a negative control. Cell pellets were collected 24 hours post-transduction and processed for nucleic acid isolation. Vector genome copy number and HSV-ATP2C1-encoded codon-optimized human ATP2C1 transcript expression were quantified via quantitative polymerase chain reaction (qPCR) and quantitative reverse transcription polymerase chain reaction (qRT-PCR), respectively. As shown in FIG. 2, HaCaT cells were found to be efficiently transduced by the HSV-ATP2C1 vector (FIG. 2A), leading to robust, dose-dependent expression of the human transgene (FIG. 2B).

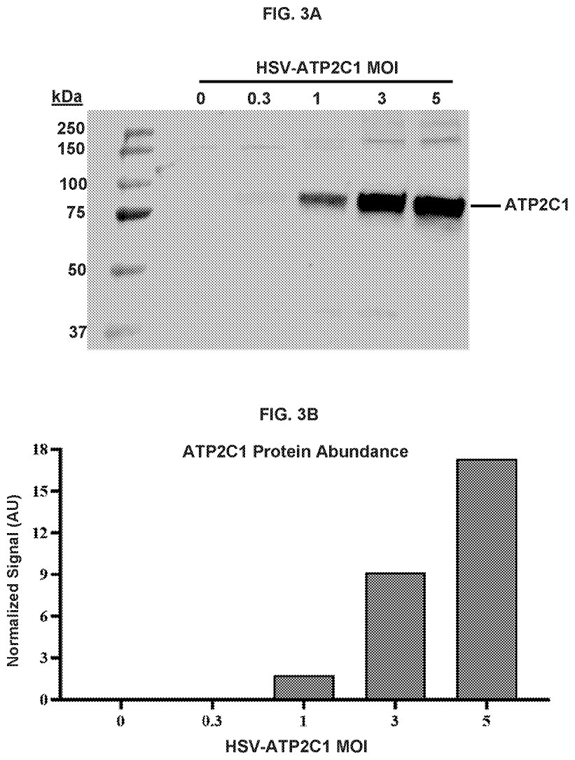

Next, HSV-ATP2C1 was tested to confirm its ability to produce full-length ATP2C1 protein with an expected size of ~95 kilodaltons (kDa) via western blot (WB). HaCaT cells were mock infected (MOI 0) or transduced with HSV-ATP2C1 at MOIs of 0.3, 1, 3, or 5, and cell pellets were collected 24 hours post-transduction and assessed for human ATP2C1 protein expression (FIG. 3A). Transduction of HSV-ATP2C1 in HaCaT cells resulted in expression of full-length ATP2C1 protein in a dose-dependent manner (FIG. 3B).

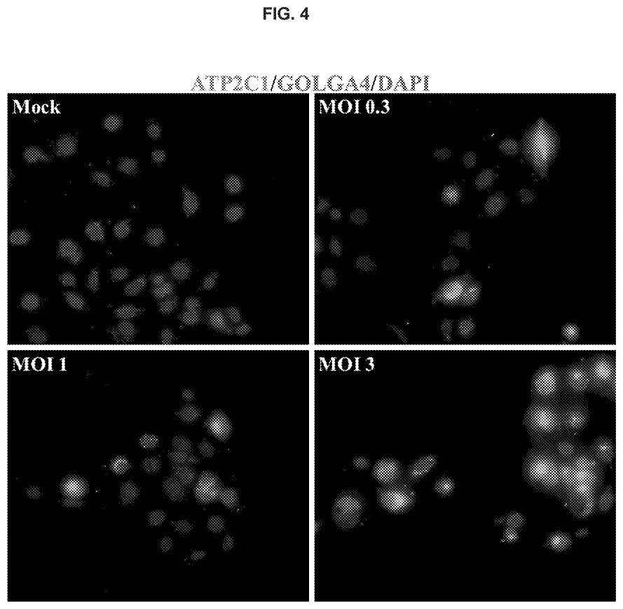

Proper localization of HSV-ATP2C1-encoded ATP2C1 protein to the Golgi apparatus was then confirmed via immunofluorescence. HaCaT cells were grown on chamber slides and transduced with HSV-ATP2C1 at MOIs of 0.3, 1, and 3. Mock-transduced cells (MOI 0) were used as a negative control. After 24 hours, cells were fixed with 4% formaldehyde and incubated with antibodies against ATP2C1 and GOLGA4, a marker for the Golgi apparatus. As shown in FIG. 4, immunofluorescence imaging confirmed that ATP2C1 protein produced by HSV-ATP2C1 colocalized with GOLGA4.

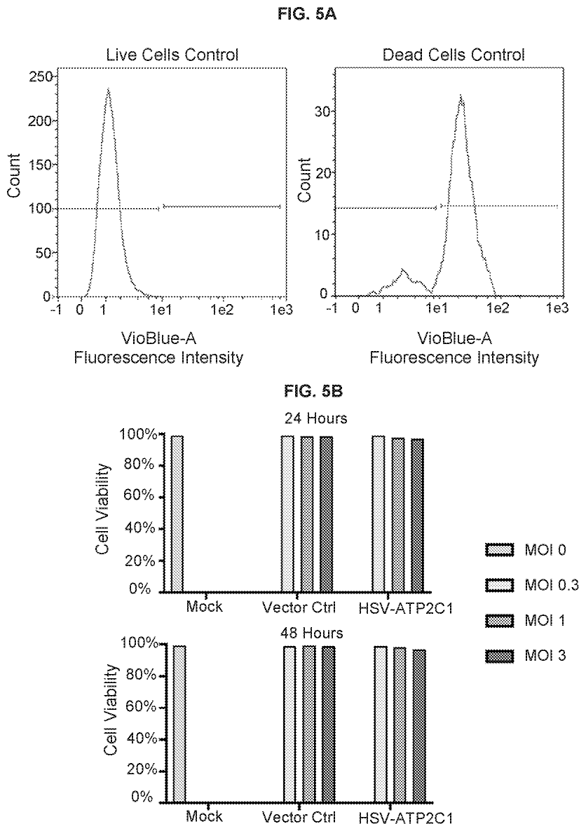

To investigate the potential cytotoxic effects of the recombinant virus, dose-dependent toxicity of HSV-ATP2C1 was evaluated by a live/dead flow cytometry assay using Viobility 405/452 fixable dyes. Viobility dye is unable to penetrate the plasma membrane of live cells but can pass through the compromised membrane of dead cells, greatly increasing fluorescence when bound to intracellular proteins. For this study, intensity thresholds were set by measuring the fluorescence of unstained HaCaT cells (live) versus those that were treated on ice with 70% ethanol for 30 minutes (dead) (FIG. 5A). No significant dose-dependent effects on cell viability were observed 24 or 48 hours after transduction with HSV-ATP2C1 in clinically-relevant human keratinocytes, as compared to either a vector control (comprising the same HSV-1 backbone but lacking the ATP2C1 transgene) or compared to mock-transduced (MOI 0) cells (FIG. 5B).

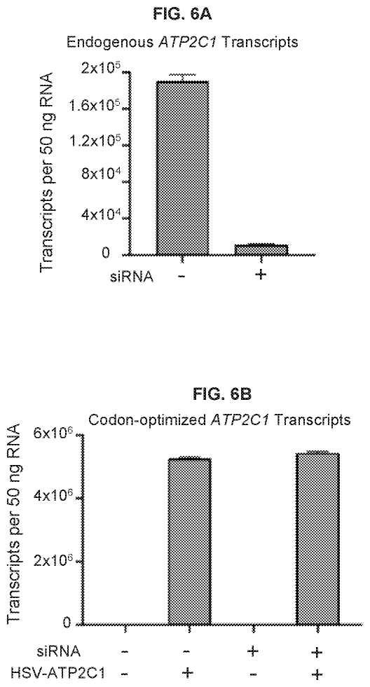

The ability of HSV-ATP2C1 to correct functional deficiencies was next tested in human keratinocytes. First, HaCaT cells were treated with a small interfering RNA (siRNA) targeting endogenous ATP2C1 transcripts, and RNA was extracted from the cells 24 hours after treatment. qRT-PCR was performed to confirm knockdown of wild-type (WT) ATP2C1 transcripts in siRNA-treated versus untreated cells, which demonstrated that siRNA reduced endogenous ATP2C1 transcripts by approximately 94% (FIG. 6A). To confirm the ability of HSV-ATP2C1 to supplement ATP2C1 expression in cells, ATP2C1 transcripts were analyzed using primers specifically designed to bind the codon-optimized ATP2C1 sequence delivered by HSV-ATP2C1 and not the endogenous WT sequence. Codon-optimized ATP2C1 transcripts were significantly increased in transduced cells, in both untreated and siRNA-treated cells, while codon-optimized ATP2C1 was not detected in non-transduced cells (FIG. 6B). These data indicated that siRNA against ATP2C1 was effective at knocking down transcript expression, while HSV-ATP2C1 promoted expression of ATP2C1 in siRNA-treated HaCaT cells.

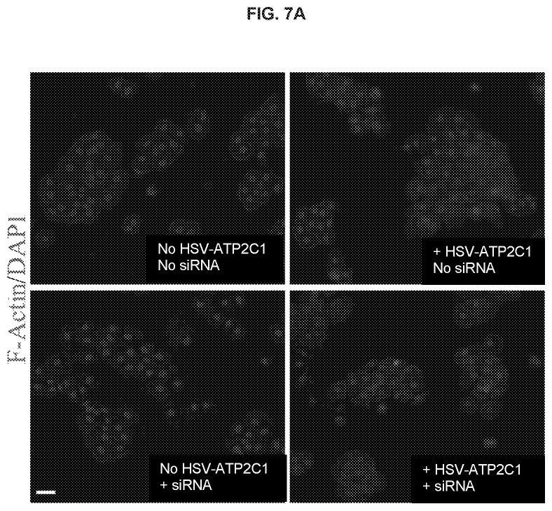

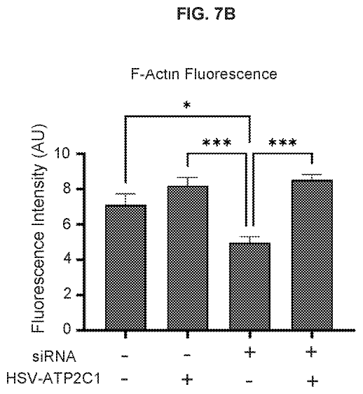

HaCaT cells with siRNA-mediated knockdown of ATP2C1 were next used to investigate the ability of HSV-ATP2C1 to provide functional correction of ATP2C1 deficiencies in vitro. Previous studies demonstrated that the filamentous cytoskeletal protein actin (F-actin) is reduced in keratinocytes after the loss of ATP2C1. In line with these findings, the ability of HSV-ATP2C1 to reverse F-actin loss in ATP2C1-deficient keratinocytes was investigated via immunofluorescence. HaCaT cells were treated with a combination of siRNA and HSV-ATP2C1 at an MOI of 1 and fixed with 4% formaldehyde 24 hours after treatment. Fixed cells were then stained with fluorescent phalloidin (which selectively binds to F-actin) and imaged under a fluorescence microscope (FIG. 7A). F-actin intensity across multiple fields was quantified using ImageJ (FIG. 7B) indicating that HSV-ATP2C1 treatment reversed F-actin loss induced by ATP2C1 knockdown.

### In Vivo

Next, several pharmacodynamic studies were conducted in mice utilizing the replication-defective HSV-ATP2C1 described above. A mouse model of Hailey-Hailey Disease is not readily available, as homozygous null mutations in ATP2C1 are embryonic lethal, and mice with heterozygous mutations show different phenotypes than those observed in humans. Therefore, wild-type C57BL/6 mice were used to assess the pharmacologic properties of HSV-ATP2C1 in vivo. The main objectives of these studies were to: (1) quantify vector genome deposition, ATP2C1 transcript expression, and protein delivery after a single topical dose of HSV-ATP2C1; and (2) assess the safety profile of topically applied HSV-ATP2C1 at the highest achievable dose.

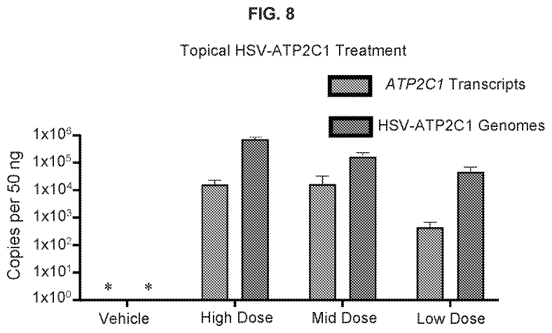

First, the ability of HSV-ATP2C1 to successfully transduce dermal keratinocytes in vivo in a dose-dependent manner was explored in healthy C57BL/6 mice via topical vector administration. Mice received a single topical administration of one of three different concentrations of HSV-AP2C1 (High: 2.2×10⁸ plaque forming units (PFU), Mid: 7.3×10⁷ PFU, Low: 2.4×10⁷ PFU per site) to abraded skin in the dorsal thoracic region. Full-thickness skin punch biopsies were collected 24 hours post-treatment and were analyzed for HSV-ATP2C1 genome copies and human ATP2C1 transcripts via qPCR and qRT-PCR, respectively. Samples from mice treated with vehicle only (5% glycerol in Dulbecco's phosphate-buffered saline (DPBS)) were used as a control. Topical administration of HSV-ATP2C1 resulted in dose-dependent detection of HSV-ATP2C1 genomes and human ATP2C transcripts in the skin, while all samples from vehicle control animals were below the limit of quantification (FIG. 8). Blood was collected to assess systemic distribution of the vector from the treatment site. Data showed that genome copies in the blood of all animals were at or below the limit of detection, with the exception of a single mid-dose animal that was found to be within the low-detectable range (fewer than 250 genome copies per 50 ng DNA), thus indicating that HSV-ATP2C1 remained constrained to the skin after topical administration.

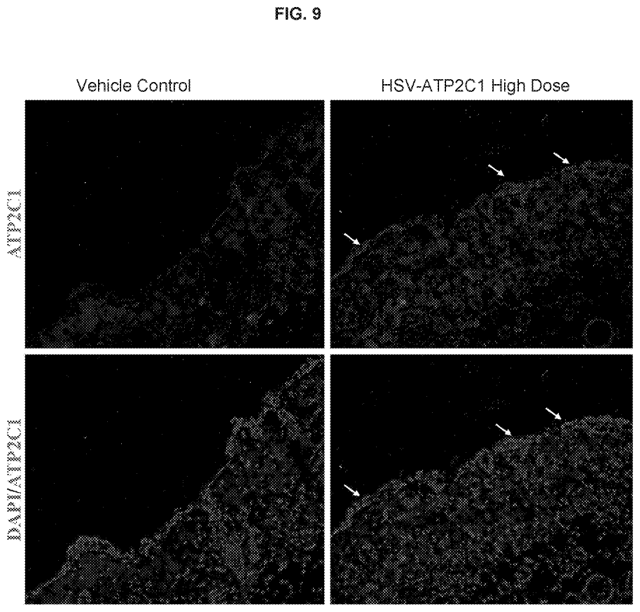

The dermal localization of human ATP2C1 protein was then assessed via immunofluorescence in topically treated skin (FIG. 9). All vehicle control-treated skin samples showed only background staining, while HSV-ATP2C1 treated samples demonstrated strong ATP2C1 fluorescent signals visible within the epidermis 24 hours post-administration.

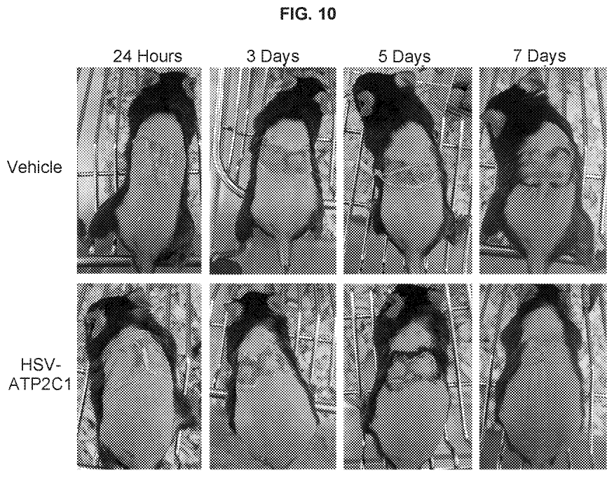

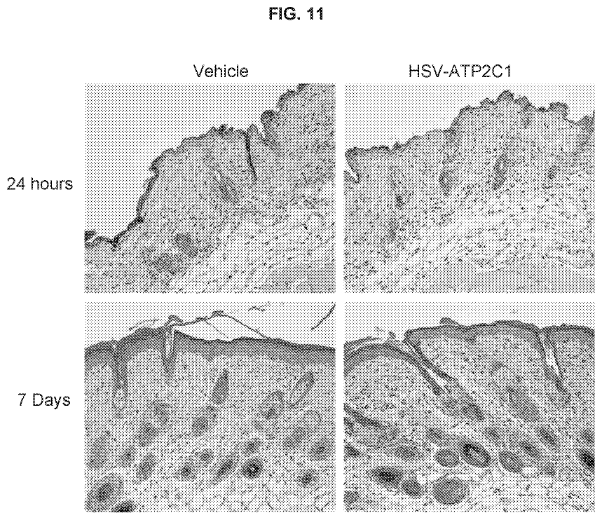

Next, the safety of HSV-ATP2C1 was evaluated in healthy C57BL/6 mice after topical administration of the maximum achievable HSV-ATP2C1 dose (4.135×10⁸ PFU per site, based on the titer of the particular batch employed in this study) on two sites in the dorsal thoracic region after depilation and tape-stripping. Administration sites were monitored to assess gross physiological reactions to topical HSV-ATP2C1 versus vehicle control up to one week after dosing. The dose sites of HSV-ATP2C1-treated mice did not look significantly different from the vehicle control-treated mice; scabbing and redness were consistent between groups, likely resulting from tape stripping and the use of surgical adhesive that was necessary for the topical administration protocol (FIG. 10). Full-thickness skin punch biopsies were also collected 24 hours and 7 days after treatment for histological analysis (FIG. 11). Data showed that HSV-ATP2C1 was well-tolerated, with minimal-to-mild inflammatory infiltrate observed in both the vehicle- and HSV-ATP2C1-treated groups, and no vector specific findings or signs of necrosis.

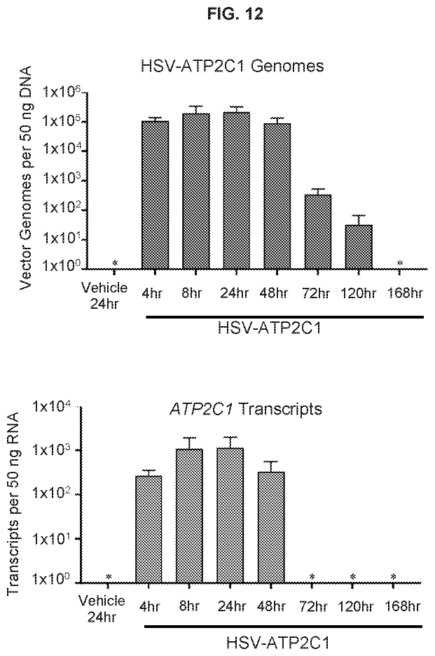

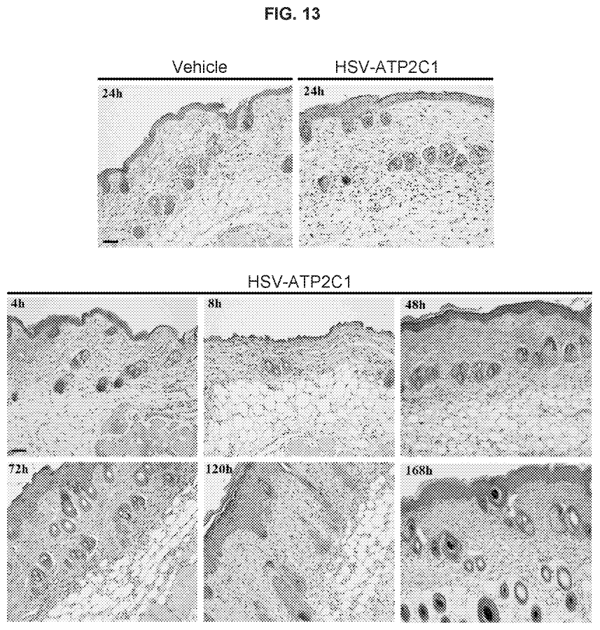

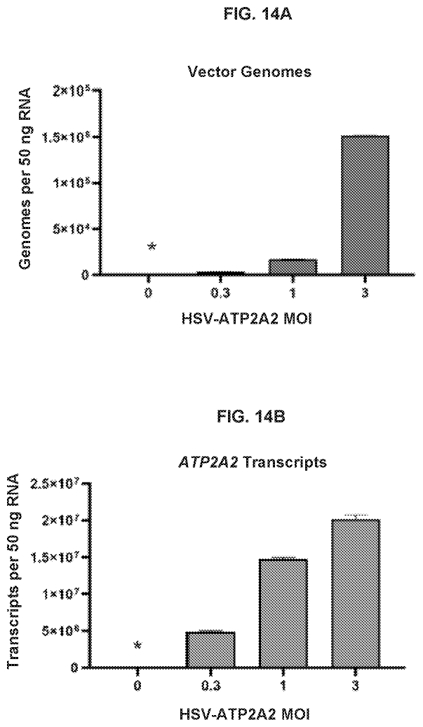

Next, a pharmacokinetic evaluation of HSV-ATP2C1 following topical administration to healthy mice was performed. To determine the pharmacokinetics of HSV-ATP2C1, healthy mice were treated with a single dose of HSV-ATP2C1 (4.135×10⁸ PFU/site) administered topically to the dorsal thoracic region. Full-thickness skin punch biopsies were collected over time post-treatment and were analyzed for HSV-ATP2C1 vector genome copies and human ATP2C1 transcripts via qPCR and qRT-PCR, respectively. Samples from mice treated with vehicle only (5% glycerol in DPBS) were used as controls. As shown in FIG. 12, topical administration of HSV-ATP2C1 resulted in immediate and robust HSV-ATP2C1 genome deposition and ATP2C1 expression, which peaked around 24 hours. HSV-ATP2C1 genomes and transcripts then reduced over time to undetectable levels after 168 hours (7 days) post-dose (FIG. 12). Full-thickness skin punch biopsies were also collected at each time point for histological analysis. As shown in FIG. 13, the highest achievable dose was well-tolerated at all tested time points. Few and scattered lymphocytes were observed in the dermis of both vehicle and HSV-ATP2C1-treated groups. As previously observed, there were no vector-specific pathological findings or signs of necrosis.

These in vitro and in vivo data indicate that: (1) HSV-ATP2C1 capably transduced clinically-relevant human epidermal keratinocytes and induced expression of full-length human ATP2C1; (2) the vector was found to be noncytotoxic to cells in culture; (3) the vector could be effectively delivered to skin in vivo via topical administration leading to robust ATP2C1 expression; and (4) HSV-ATP2C1 administration resulted in no gross pathological changes or necrosis at all tested timepoints, demonstrating a potentially favorable safety profile. Taken together, these observations support the application of HSV-ATP2C1 as a promising, innovative, and noninvasive HSV-1-based therapy to treat Hailey-Hailey Disease.

### Example 3: In Vitro and In Vivo Analysis of a Modified Herpes Simplex Virus Encoding Human Sarcoplasmic/Endoplasmic Reticulum Calcium ATPase 2 (ATP2A2) Polypeptide

Darier disease is characterized by mutations in the ATP2A2 gene which encodes a calcium pump termed Sarcoplasmic/endoplasmic reticulum calcium ATPase 2 (also referred to as “SERCA2” or “SR Ca²⁺-ATPase2”) resulting in impaired calcium homeostasis in keratinocytes and decreased cell-cell adhesion. Individuals with Darier disease are characterized by reddish brown, keratotic papules in seborrheic and intertriginous areas, which may coalesce into extensive lesions. Individuals with Darier disease have significantly reduced quality of life due to, for example, itching, burning sensation, pain, and body malodor. Therapeutic options remain limited, and thus, there exists a need for more effective therapies. The use of an HSV-1 based vector platform technology provides an innovative treatment strategy for Darier Disease by providing functional full-length Sarcoplasmic/endoplasmic reticulum calcium ATPase 2 protein directly to the affected areas of the skin. The use of an HSV-1-based vector both capable of packaging full-length human ATP2A2 and possessing a natural tropism to human skin would therefore represent an innovative strategy for the treatment of Darier Disease, where topical application would lead to robust expression of Sarcoplasmic/endoplasmic reticulum calcium ATPase 2 protein in epidermal keratinocytes, thereby molecularly correcting the underlying disease state.

This study coupled the beneficial properties inherent to HSV-1, including its episomal lifecycle, high payload capacity, transduction efficiency, tropism for myriad human cell types (including those of the skin), and natural immune evasiveness, with a genetic modification strategy of targeted immediate early (IE) gene deletions to render a vector platform that is both replication-defective and non-cytotoxic. Accordingly, described herein is the use of a replication-defective, non-integrating HSV-1 based vector that has been engineered to deliver functional, full-length Sarcoplasmic/endoplasmic reticulum calcium ATPase 2 (termed HSV-ATP2A2) directly to the skin via topical administration to provide an effective redosable therapy for Darier Disease. In vitro

The HSV-ATP2A2 vector was first tested for its ability to transduce and deliver its transgene to human cells. HaCaT cells were used as a clinically relevant model to verify both HSV-ATP2A2 transduction efficiency and ATP2A2 expression. Towards this end, cells were transduced with HSV-ATP2A2 at multiplicities of infection (MOIs) of 0.3, 1, and 3. Mock-transduced cells (MOI 0) were used as a negative control. Cell pellets were collected post-transduction and processed for nucleic acid isolation. Vector genome copy number and HSV-ATP2C1-encoded codon-optimized human ATP2C1 transcript expression were quantified via quantitative polymerase chain reaction (qPCR) and quantitative reverse transcription polymerase chain reaction (qRT-PCR), respectively. As shown in FIG. 14, HaCaT cells were found to be efficiently transduced by the HSV-ATP2A2 vector (FIG. 14A), leading to robust, dose-dependent expression of the human transgene (FIG. 14B).

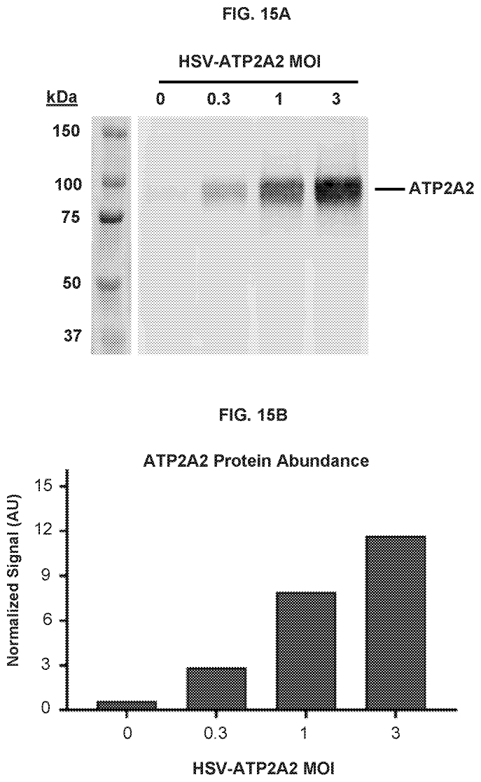

Next, HSV-ATP2A2 was tested to confirm its ability to produce full-length ATP2A2 protein via western blot (WB). HaCaT cells were mock infected (MOI 0) or transduced with HSV-ATP2A2 at MOIs of 0.3, 1, or 3, and cell pellets were collected post-transduction and assessed for human ATP2A2 protein expression (FIG. 15A). Transduction of HSV-ATP2A2 in HaCaT cells resulted in expression of full-length ATP2A2 protein in a dose-dependent manner (FIG. 15B).

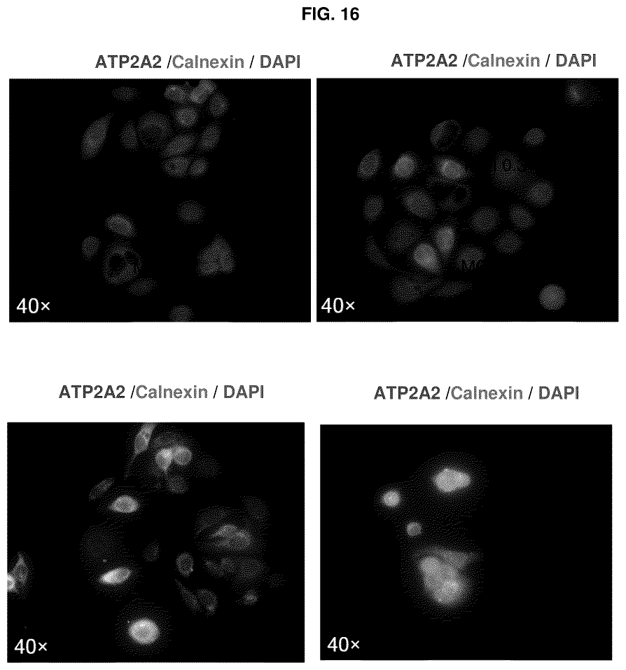

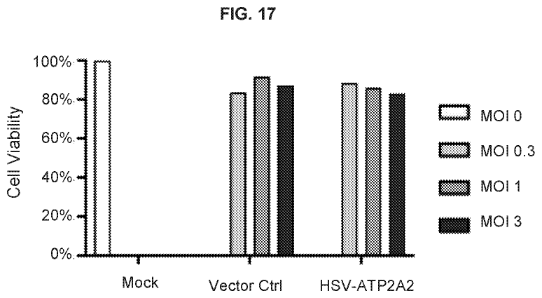

Proper localization of HSV-ATP2A2-encoded ATP2A2 protein to the endoplasmic reticulum was then confirmed via immunofluorescence. HaCaT cells were grown on chamber slides and transduced with HSV-ATP2A2 at MOIs of 0.3, 1, and 3. Mock-transduced cells (MOI 0) were used as a negative control. Following transduction, cells were fixed with 4% formaldehyde and incubated with antibodies against ATP2A2 and Calnexin, a marker for the endoplasmic reticulum. As shown in FIG. 16, immunofluorescence imaging confirmed that ATP2A2 protein produced by HSV-ATP2A2 colocalized with Calnexin.

To investigate the potential cytotoxic effects of the recombinant virus, dose-dependent toxicity of HSV-ATP2A2 was evaluated by a Mosmann's Tetrazolium Toxicity assay. No significant dose-dependent effects on cell viability were observed 48 hours after transduction with HSV-ATP2A2 in clinically-relevant human keratinocytes, as compared to either a vector control (comprising the same HSV-1 backbone but lacking the ATP2A2 transgene) or to mock-transduced (MOI 0) cells (FIG. 17).

In vivo

Next, pharmacodynamic studies were conducted in mice utilizing the replication-defective HSV-ATP2A2 described above. Wild-type C57BL/6 mice were used to assess the pharmacologic properties of HSV-ATP2A2 in vivo. The main objectives of these studies were to quantify vector genome deposition, ATP2C1 transcript expression, and protein delivery after a single topical dose of HSV-ATP2A2.

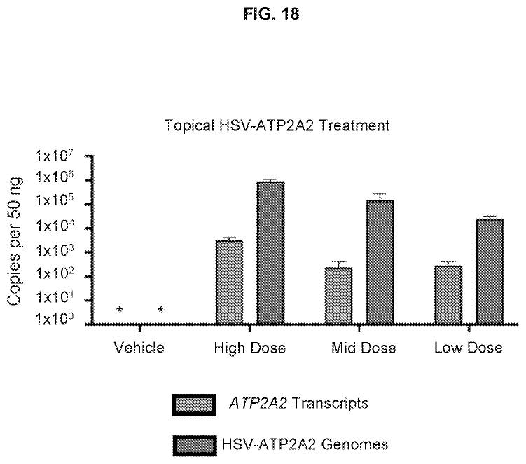

First, the ability of HSV-ATP2A2 to successfully transduce dermal keratinocytes in vivo in a dose-dependent manner was explored in healthy C57BL/6 mice via topical vector administration. Mice received a single topical administration of one of three different concentrations of HSV-ATP2A2 (high, mid, and low dose) to abraded skin in the dorsal thoracic region. Full-thickness skin punch biopsies were collected 24 hours post-treatment and were analyzed for HSV-ATP2A2 genome copies and human ATP2A2 transcripts via qPCR and qRT-PCR, respectively. Samples from mice treated with vehicle only (5% glycerol in Dulbecco's phosphate-buffered saline (DPBS)) were used as a control. Topical administration of HSV-ATP2A2 resulted in dose-dependent detection of HSV-ATP2A2 genomes and human ATP2A2 transcripts in the skin, while all samples from vehicle control animals were below the limit of quantification (FIG. 18).

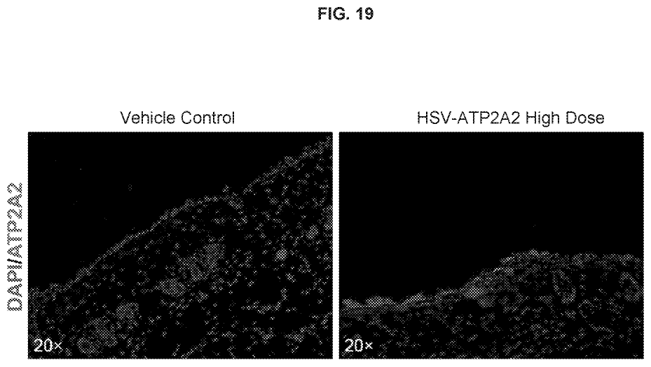

The dermal localization of human ATP2A2 protein was then assessed via immunofluorescence in topically treated skin (FIG. 19). All vehicle control-treated skin samples showed only background staining (data not shown), while HSV-ATP2A2 treated samples demonstrated strong ATP2A2 fluorescent signals visible within the epidermis following HSV-ATP2A2 administration.

These in vitro and in vivo data indicate that: (1) HSV-ATP2A2 capably transduced clinically-relevant human epidermal keratinocytes and induced expression of full-length human ATP2A2; (2) the vector was found to be noncytotoxic to cells in culture; and (3) the vector could be effectively delivered to skin in vivo via topical administration leading to robust ATP2A2 expression. Taken together, these observations support the application of HSV-ATP2A2 as a promising, innovative, and noninvasive HSV-1-based therapy to treat Darier Disease.

1.-72. (canceled)

73. A pharmaceutical composition comprising:
(a) a replication-defective herpes simplex virus comprising a recombinant herpes simplex virus genome, wherein the recombinant herpes simplex virus genome comprises a polynucleotide encoding a calcium-transporting ATPase type 2C member 1 (ATP2C1) polypeptide; and
(b) a pharmaceutically acceptable excipient.

74. The pharmaceutical composition of claim 73, wherein the recombinant herpes simplex virus genome is a recombinant herpes simplex virus type 1 (HSV-1) genome or a recombinant herpes simplex virus type 2 (HSV-2) genome.

75. The pharmaceutical composition of claim 73, wherein the recombinant herpes simplex virus genome is a recombinant HSV-1 genome.

76. The pharmaceutical composition of claim 73, wherein the recombinant herpes simplex virus genome comprises an inactivating mutation in a herpes simplex virus gene selected from the group consisting of Infected Cell Protein (ICP) 0, ICP4, ICP22, ICP27, ICP47, thymidine kinase (tk), Long Unique Region (UL) 41, and UL55.

77. The pharmaceutical composition of claim 73, wherein the recombinant herpes simplex virus genome comprises an inactivating mutation in one or both copies of the ICP4 gene.

78. The pharmaceutical composition of claim 73, wherein the recombinant herpes simplex virus genome comprises an inactivating mutation in the ICP22 gene.

79. The pharmaceutical composition of claim 73, wherein the recombinant herpes simplex virus genome comprises the polynucleotide encoding the ATP2C1 polypeptide within one or both of the ICP4 viral gene loci.

80. The pharmaceutical composition of claim 73, wherein the recombinant herpes simplex virus genome comprises the polynucleotide encoding the ATP2C1 polypeptide within the ICP22 viral gene locus.

81. The pharmaceutical composition of claim 73, wherein the ATP2C1 polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 1.

82. The pharmaceutical composition of claim 73, wherein the ATP2C1 polypeptide is a human ATP2C1 polypeptide.

83. The pharmaceutical composition of claim 73, wherein the pharmaceutical composition is suitable for topical, transdermal, subcutaneous, intradermal, oral, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, injection, or epicutaneous administration.

84. The pharmaceutical composition of claim 73, wherein the pharmaceutical composition is suitable for topical administration.

85. A pharmaceutical composition comprising:
(a) a replication-defective herpes simplex virus comprising a recombinant herpes simplex virus genome, wherein the recombinant herpes simplex virus genome comprises a polynucleotide encoding a sarcoplasmic/endoplasmic reticulum calcium ATPase 2 (ATP2A2) polypeptide; and
(b) a pharmaceutically acceptable excipient.

86. The pharmaceutical composition of claim 85, wherein the recombinant herpes simplex virus genome is a recombinant HSV-1 genome or a recombinant HSV-2 genome.

87. The pharmaceutical composition of claim 85, wherein the recombinant herpes simplex virus genome comprises an inactivating mutation in a herpes simplex virus gene selected from the group consisting of ICPO, ICP4, ICP22, ICP27, ICP47, tk, UL41, and UL55.

88. The pharmaceutical composition of claim 85, wherein the recombinant herpes simplex virus genome comprises an inactivating mutation in one or both copies of the ICP4 gene.

89. The pharmaceutical composition of claim 85, wherein the ATP2A2 polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 2.

90. The pharmaceutical composition of claim 85, wherein the pharmaceutical composition is suitable for topical, transdermal, subcutaneous, intradermal, oral, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, injection, or epicutaneous administration.

91. The pharmaceutical composition of claim 85, wherein the pharmaceutical composition is suitable for topical administration.

92. A method of providing prophylactic, palliative, or therapeutic relief of one or more signs or symptoms of Darier disease in a subject in need thereof, the method comprising administering to the subject an effective amount of a pharmaceutical composition comprising:
(a) a replication-defective herpes simplex virus comprising a recombinant herpes simplex virus genome, wherein the recombinant herpes simplex virus genome comprises a polynucleotide encoding an ATP2A2 polypeptide; and
(b) a pharmaceutically acceptable excipient.

93. The method of claim 92, wherein the pharmaceutical composition is administered via injection, topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, intraarticularly, periarticularly, locally, or via inhalation to the subject.

94. The method of claim 92, wherein the pharmaceutical composition is administered topically to the subject.

95. The method of claim 92, wherein the recombinant herpes simplex virus genome is a recombinant HSV-1 genome or a recombinant HSV-2 genome.

96. The method of claim 92, wherein the recombinant herpes simplex virus genome comprises an inactivating mutation in a herpes simplex virus gene selected from the group consisting of ICPO, ICP4, ICP22, ICP27, ICP47, tk, UL41, and UL55.

97. The method of claim 92, wherein the recombinant herpes simplex virus genome comprises an inactivating mutation in one or both copies of the ICP4 gene.

98. The method of claim 92, wherein the ATP2A2 polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 2.
